(19) United States
(12) Patent Application Publication
(10) Pub. No.: US 2024/0352133 A1
(43) Pub. Date: Oct. 24, 2024

(54) COMPOSITIONS AND METHODS FOR TREATMENT OF THYROID EYE DISEASE

(71) Applicant: Viridian Therapeutics, Inc., Waltham, MA (US)

(72) Inventors: Vahe BEDIAN, Waltham, MA (US); Yang ZHAO, Waltham, MA (US)

(73) Assignee: Viridian Therapeutics, Inc., Waltham, MA (US)

(21) Appl. No.: 18/749,821

(22) Filed: Jun. 21, 2024

Related U.S. Application Data
(63) Continuation of application No. PCT/US2022/082214, filed on Dec. 22, 2022.
(60) Provisional application No. 63/293,242, filed on Dec. 23, 2021.

(51) Int. Cl. C07K 16/28; A61K 33/04; A61K 39/00; A61K 39/395; A61K 45/06; A61P 27/02; C07K 16/24

(52) U.S. Cl. CPC C07K 16/2863; A61K 33/04; A61K 39/3955; A61K 45/06; A61P 27/02; C07K 16/241; C07K 16/248; C07K 16/2887; A61K 2039/54; A61K 2039/545; C07K 2317/24; C07K 2317/622

(57) ABSTRACT

Antibodies and compositions against IGF-1R and uses thereof are provided herein.

### CROSS-REFERENCE TO RELATED APPLICATIONS

This application is a continuation of International Application No. PCT/US2022/082214, filed Dec. 22, 2022, which claims priority to U.S. Provisional Application No. 63/293,242, filed Dec. 23, 2021, each of which are hereby incorporated by reference in their entireties.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on Jun. 20, 2024, is named “VRD-006US1_SL.XML” and is 401,799 bytes in size.

### BACKGROUND

Thyroid-associated ophthalmopathy (TAO), also known as thyroid eye disease (TED), Graves' ophthalmopathy or orbitopathy (GO), thyrotoxic exophthalmos, dysthyroid ophthalmopathy, and several other terms, is orbitopathy associated with thyroid dysfunction. TAO is divided into two types. Active TAO, which typically lasts 1-3 years, is characterized by an ongoing autoimmune/inflammatory response in the soft tissues of the orbit. Active TAO is responsible for the expansion and remodeling of the ocular soft tissues. The autoimmune/inflammatory response of active TAO spontaneously resolves and the condition transitions into inactive TAO. Inactive TAO is the term used to describe the long-term/permanent sequelae of active TAO. The cause of TAO is unknown. TAO is typically associated with Graves' hyperthyroidism, but can also occur as part of other autoimmune conditions that affect the thyroid gland and produce pathology in orbital and periorbital tissue, and, rarely, the pretibial skin (pretibial myxedema) or digits (thyroid acropachy). TAO is an autoimmune orbitopathy in which the orbital and periocular soft tissues are primarily affected with secondary effects on the eye and vision. In TAO, as a result of inflammation and expansion of orbital soft tissues, primarily eye muscles and adipose, the eyes are forced forward (bulge) out of their sockets—a phenomenon termed proptosis or exophthalmos. Although most cases of TAO do not result in loss of vision, this condition can cause vision-threatening exposure keratopathy, troublesome diplopia (double vision), and compressive dysthyroid optic neuropathy. TAO may precede, coincide with, or follow the systemic complications of dysthyroidism. The ocular manifestations of TAO include upper eyelid retraction, lid lag, swelling, redness (erythema), conjunctivitis, and bulging eyes (exophthalmos or proptosis), chemosis, periorbital edema, and altered ocular motility with significant functional, social, and cosmetic consequences. Many of the signs and symptoms of TAO, including proptosis and ocular congestion, result from expansion of the orbital adipose tissue and periocular muscles. The adipose tissue volume increases owing in part to new fat cell development (adipogenesis) within the orbital fat. The accumulation of hydrophilic glycosaminoglycans, primarily hyaluronic acid, within the orbital adipose tissue and the perimysial connective tissue between the extraocular muscle fibers, further expands the fat compartments and enlarges the extraocular muscle bodies. Hyaluronic acid is produced by fibroblasts residing within the orbital fat and extraocular muscles, and its synthesis in vitro is stimulated by several cytokines and growth factors, including IL-1beta, interferon-gamma, platelet-derived growth factor, thyroid stimulating hormone (TSH) and insulin-like growth factor I (IGF-I).

Antibodies that activate the insulin-like growth factor I receptor (IGF-IR) have also been detected and implicated in active TAO. Without being bound to any theory, it is believed that TSHR and IGF-IR form a physical and functional complex in orbital fibroblasts, and that blocking IGF-IR appears to attenuate both IGF-1 and TSH-dependent signaling. It has been suggested that blocking IGF-IR using an antibody antagonist might reduce both TSHR- and IGF-I-dependent signaling and therefore interrupt the pathological activities of autoantibodies acting as agonists on either receptor.

IGF-IR is a widely expressed heterotetrameric protein involved in the regulation of proliferation and metabolic function of many cell types. It is a tyrosine kinase receptor comprising two subunits. IGF-IRalpha contains a ligand-binding domain while IGF-IRbeta is involved in signaling and contains tyrosine phosphorylation sites.

Current therapies for hyperthyroidism due to Graves' disease are imperfect because therapies targeting the specific underlying pathogenic autoimmune mechanisms of the disease are lacking. Even more complex is the treatment of moderate-to-severe active TAO. Although recent years have witnessed a better understanding of its pathogenesis, TAO remains a therapeutic challenge and dilemma. There are no approved drugs to treat active TAO. Intravenous glucocorticoids (ivGCs) and oral glucocorticoids are used to treat patients with moderate-to-severe active TAO, but results are seldom satisfactory. Partial responses are frequent and relapses (rebound) after drug withdrawal are not uncommon. Adverse events do occur and many patients eventually require rehabilitative surgery conducted when their condition has transitioned to inactive TAO. Accordingly, there is still a need to provide alternative therapies for TAO and its related symptoms.

### SUMMARY

An antibody, or antigen binding fragment thereof, is provided herein. In some embodiments, the antibody, or antigen binding fragment thereof, comprises a heavy chain complementarity-determining region sequence selected from the group consisting of SEQ ID NO: 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, and 321; and a light chain CDR sequence selected from the group consisting of SEQ ID NO: 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, and 362.

In some embodiments, an antibody, or antigen binding fragment thereof, is provided herein comprising a heavy chain variable region (VH) sequence selected from the group consisting of SEQ ID NO: 1, 3, 4, 5, 6, 7, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 67, 68, 69, 70, 71, 72, 73, 74, 75, 76, 77, 78, 79, 80, 81, 82, 83, 84, 85, 86, 87, 88, 99, 101, 102, 103, 104, 105, 106, 107, 108, 110, 111, 112, 113, 114, 115, 116, 117, 118, 119, 120, and 121; and a light chain variable region (VL) sequence selected from the group consisting of SEQ ID NO: 2, 8, 39, 40, 41, 42, 43, 44, 45, 46, 47, 48, 49, 50, 51, 52, 53, 54, 55, 56, 57, 58, 59, 60, 61, 62, 63, 64, 65, 66, 89, 90, 91, 92, 93, 94, 95, 96, 97, 98, 100, 109, and 122.

In some embodiments, any antibody or antigen binding fragment thereof provided herein binds to insulin-like growth factor I receptor (IGF-1R). In some embodiments, the antibody is a monoclonal antibody. In some embodiments, the antibody is a humanized antibody. In some embodiments, the antibody is a scFv antibody.

In some embodiments, the antibody, or antigen binding fragment thereof, comprises a VH peptide comprising an amino acid sequence as set forth in SEQ ID NOs: 1, 3, 4, 5, 6, 7, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 67, 68, 69, 70, 71, 72, 73, 74, 75, 76, 77, 78, 79, 80, 81, 82, 83, 84, 85, 86, 87, 88, 99, 101, 102, 103, 104, 105, 106, 107, 108, 110, 111, 112, 113, 114, 115, 116, 117, 118, 119, 120, or 121, or any variant thereof.

In some embodiments, the antibody, or antigen binding fragment thereof, comprises a VL peptide comprising an amino acid sequence as set forth in SEQ ID NOs: 2, 8, 39, 40, 41, 42, 43, 44, 45, 46, 47, 48, 49, 50, 51, 52, 53, 54, 55, 56, 57, 58, 59, 60, 61, 62, 63, 64, 65, 66, 89, 90, 91, 92, 93, 94, 95, 96, 97, 98, 100, 109, or 122, or any variant thereof.

In some embodiments, an antibody, or antigen binding fragment thereof, is provided, comprising: (i) a heavy chain variable region comprising HCDR1, HCDR2, and HCDR3 sequences, wherein the HCDR1 sequence has the amino acid sequence of SEQ ID NO: 263, 264, 265, 266, 267, 268, 269, 270, 271, 272, 273, 274, 275, or 276; the HCDR2 has the amino acid sequence of SEQ ID NO: 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, or 397; and the HCDR3 sequence has the amino acid sequence of SEQ ID NO: 311, 312, 313, 314, 315, 316, 317, 318, 319, 320, or 321; or variants of any of the foregoing; and (ii) a light chain variable region comprising LCDR1, LCDR2, and LCDR3 sequences, wherein the LCDR1 sequence has the amino acid sequence SEQ ID NO: 322, 323, 324, 325, 326, 327, 328, 329, 330, 331, 332, 333, 334, 335, 336, 337, or 338; the LCDR2 sequence has the amino acid sequence of SEQ ID NO: 339, 340, 341, 342, 343, 344, 345, 346, 347, 348, 349, or 350; and the LCDR3 sequence has the amino acid sequence of SEQ ID NO: 351, 352, 353, 354, 355, 356, 357, 358, 359, 360, 361, or 362; or variants of any of the foregoing.

In some embodiments, an antibody, or antigen binding fragment thereof, is provided comprising a VH and VL pair comprising the amino acid sequence of SEQ ID NOs:123-262, or a variant thereof.

In some embodiments, a method of treating or reducing the severity of, thyroid-associated ophthalmopathy (TAO), or a symptom thereof is provided, comprising administering to a subject any antibody disclosed herein or any pharmaceutical composition disclosed herein.

In some embodiments, a method of reducing proptosis in an eye in a subject with thyroid-associated ophthalmopathy (TAO) is provided, comprising administering to a subject any antibody disclosed herein or any pharmaceutical composition disclosed herein.

In some embodiments, a method of treating thyroid eye disease in a subject is provided, comprising administering to a subject an administering to a subject any antibody disclosed herein or any pharmaceutical composition disclosed herein.

In some embodiments, a method of reducing Clinical Activity Score (CAS) of thyroid-associated ophthalmopathy (TAO) in a subject is provided, comprising administering to a subject any antibody disclosed herein or any pharmaceutical composition disclosed herein.

In some embodiments, a method of a) reducing proptosis by at least 2 mm and b) reducing the clinical activity score (CAS) in a subject with thyroid-associated ophthalmopathy (TAO) is provided, comprising administering to a subject an administering to a subject any antibody disclosed herein or any pharmaceutical composition disclosed herein.

In some embodiments, a method of treating or reducing the severity of diplopia in a subject with thyroid-associated ophthalmopathy (TAO) is provided, comprising administering to a subject any antibody disclosed herein or any pharmaceutical composition disclosed herein.

In some embodiments, a method of increasing the internalization of IGF-1R on a cell is provided, the method comprising contacting the cell with any antibody disclosed herein or any pharmaceutical composition disclosed herein.

In some embodiments, a method of inhibiting IGF-1 stimulated receptor phosphorylation on a cell is provided, the method comprising contacting the cell with any antibody disclosed herein or any pharmaceutical composition disclosed herein.

In some embodiments, a method of treating thyroid eye disease in a subject is provided, the method comprising administering any antibody disclosed herein or any pharmaceutical composition disclosed herein to the subject, wherein the antibody has a serum concentration in the subject of at least, or about, 70 μg/ml, 75 μg/ml, 80 μg/ml, 85 μg/ml, 90 μg/ml, 95 μg/ml, 100 μg/ml, or 105 μg/ml at least 1, 2, or 3 week after administration.

### DETAILED DESCRIPTION

Provided herein are antibodies that bind and modulate the activity of IGF-1R. The antibodies can be used, for example, to treat thyroid eye disease.

As used herein, “Thyroid-associated Ophthalmopathy” (TAO), “Thyroid Eye Disease” (TED), “Graves' Ophthalmopathy” or “Graves' Orbitopathy” (GO) refer to the same disorder or condition and are used interchangeably. They all refer to the inflammatory orbital pathology associated with some autoimmune thyroid disorders, most commonly with “Graves' Disease” (GD), but sometimes with other diseases, e.g. Hashimoto's thyroiditis.

The terms “proptosis” and “exophthalmos” (also known as exophthalmos, exophthalmia, or exorbitism) refer to the forward projection, displacement, bulging, or protrusion of an organ. As used herein, the terms refer to the forward projection, displacement, bulging, or protrusion of the eye anteriorly out of the orbit. Proptosis and exophthalmos are considered by some of skill in the art to have the same meaning and are often used interchangeably, while others attribute subtle differences to their meanings. Exophthalmos is used by some to refer to severe proptosis; or to refer to endocrine-related proptosis. Yet others use the term exophthalmos when describing proptosis associated with the eye, in, for example, subjects with TAO (TED or GO).

As used herein, the terms “proptosis” and “exophthalmos” are used interchangeably and refer to the forward projection, displacement, bulging, or protrusion of the eye anteriorly out of the orbit. Owing to the rigid bony structure of the orbit with only anterior opening for expansion, any increase in orbital soft tissue contents taking place from the side or from behind will displace the eyeball forward. Proptosis or exophthalmos can be the result of a several disease processes including infections, inflammations, tumors, trauma, metastases, endocrine lesions, vascular diseases & extra orbital lesions. TAO (TED or GO) is currently recognized as the most common cause of proptosis in adults. Exophthalmos can be either bilateral, as is often seen in TAO (TED or GO), or unilateral (as is often seen in an orbital tumor).

Measurement of the degree of exophthalmos can be performed using, for example, an exophthalmometer, an instrument used for measuring the degree of forward displacement of the eye. The device allows measurement of the forward distance of the lateral orbital rim to the front of the cornea. Computed tomography (CT) scanning and Magnetic resonance imaging (MRI) may also be used in evaluating the degree of exophthalmos or proptosis. CT scanning is an excellent imaging modality for the diagnosis of TAO. In addition to allowing visualization of the enlarged extraocular muscles, CT scans provide the surgeon or clinician with depictions of the bony anatomy of the orbit when an orbital decompression is required. MRI, with its multi-planar and inherent contrast capabilities, provides excellent imaging of the orbital contents without the radiation exposure associated with CT scan studies. MRI provides better imaging of the optic nerve, orbital fat, and extraocular muscle, but CT scans provide better views of the bony architecture of the orbit. Orbital ultrasonography can also be a used for the diagnosis and evaluation of TAO, because it can be performed quickly and with a high degree of confidence. High reflectivity and enlargement of the extraocular muscles are assessed easily, and serial ultrasonographic examinations can also be used to assess progression or stability of the ophthalmopathy. Based on the technologies currently available, or that will become available in the future, one of skill in the art would be capable of determining the best modality for diagnosing and evaluating the extent of proptosis or exophthalmos.

As used herein, the term “antibody” refers to any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic antibody.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

A “Fab fragment” is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}1 and C_{H}2 domains of an antibody. The two heavy chain fragments are held together by two or more disulfide bonds and by hydrophobic interactions of the C_{H}3 domains.

In some embodiments, the antibodies, or antigen fragments herein, comprise a Fc region. In some embodiments, the Fc region comprises a mutation that extends the half-life of the antibody when linked to the Fc region. In some embodiments, the Fc region comprises a S228P, L235E, M252Y, S254T, T256E, M428L, N434S, L234F, P331S mutation, or any combination thereof. In some embodiments, the Fc region comprises a M252Y, S254T, and T256E mutations. A non-limiting example of a Fc region comprising the M252Y, S254T, and T256E mutations (collectively, “YTE Mutations”) can be found in a sequence of SEQ ID NO: 89. In some embodiments, the Fc region comprising the YTE Mutations comprises a sequence of SEQ ID NO: 90, which differs from SEQ ID NO: 89 by the presence of a C-terminal lysine (K) residue. The numbering of the Fc region can be according to the Kabat numbering system for the Fc region.

In some embodiments, the Fc region comprises a S228P and a L235E mutation. In some embodiments, the antibody comprises a L234F, L235E, and P331S mutation. In some embodiments, the Fc region comprises M252Y, S254T, T256E, S228P and L235E mutations. In some embodiments, the Fc region comprises S228P, L235E, M428L, and N434S mutations. In some embodiments, the Fc region comprises the M428L and N434S mutations. In some embodiments, the Fc region comprises the L234F, L235E, P331S, M252Y, S254T, and T256E mutations. Mutations in the Fc region are also described in US2007041972A1, EP2235059B1, U.S. Pat. No. 8,394,925, and Mueller et al, Mol Immunol 1997 April; 34(6):441-52, each of which is incorporated by reference in its entirety. The numbering referenced herein refers to the Kabat numbering system for the Fc region.

In some embodiments, the Fc region comprises the sequence selected from:

(SEQ ID NO: 363)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCS
VMHEALHNHYTQKSLSLSPGK;
(SEQ ID NO: 364)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCS
VMHEALHNHYTQKSLSLSPGK;
(SEQ ID NO: 365)
APPVAGPSVFLFPPKPKDTLMISRTPEVTCVVVDVSHEDPEVQFNWYVD
GVEVHNAKTKPREEQFNSTFRVVSVLTVVHQDWLNGKEYKCKVSNKGLP
APIEKTISKTKGQPREPQVYTLPPSREEMTKNQVSLTCLVKGFYPSDIA
VEWESNGQPENNYKTTPPMLDSDGSFFLYSKLTVDKSRWQQGNVFSCSV
MHEALHNHYTQKSLSLSPG;
(SEQ ID NO: 366)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPELLGGPSVFLFPPKPKDTLYITREPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPG;
(SEQ ID NO: 367)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK;
(SEQ ID NO: 368)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPG;
(SEQ ID NO: 369)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLYITREPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGOPENNYKTTPPVLDSDGSFFLYSKLT
VDKSRWQQGNVFSCSVMHEALHNHYTQKSLSLSPG;
or
(SEQ ID NO: 370)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLYITREPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

A “Fab′ fragment” contains one light chain and a portion or fragment of one heavy chain that contains the V_{H} domain and the C H1 domain and also the region between the CHI and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

The term “single-chain Fv” or “scFv” antibody refers to antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv, see Pluckthun (1994) THE PHARMACOLOGY OF MONOCLONAL ANTIBODIES, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

A “bivalent antibody” comprises two antigen binding sites. In some instances, the two binding sites have the same antigen specificities. However, bivalent antibodies may be bispecific (see below).

In certain embodiments, monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079). In one embodiment, the present invention provides single domain antibodies comprising two V_{H} domains with modifications such that single domain antibodies are formed.

As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

Typically, a variant antibody or antigen binding fragment of the antibodies provided herein retain at least 10% of its IGF-1R binding activity (when compared to a parental antibody that is modified) when that activity is expressed on a molar basis. In some embodiments, a variant antibody (or antigen fragment thereof), or antigen binding fragment of an antibody provided herein, retains at least 20%, 50%, 70%, 80%, 90%, 95% or 100% or more of the IGF-1R binding affinity as the parental antibody. As described herein, it is also intended that an antibody or antigen binding fragment of the invention can include conservative or non-conservative amino acid substitutions, which can also be referred to as “conservative variants” or “function conserved variants” of the antibody, that do not substantially alter its biologic activity.

“Isolated antibody” refers to the purification status of a binding compound and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

The term “monoclonal antibody”, as used herein, refers to population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, that are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, a “chimeric antibody” is an antibody having the variable domain from a first antibody and constant domain from a second antibody, where the first and second antibodies are from different species. (U.S. Pat. No. 4,816,567; and Morrison et al., (1984) Proc. Natl. Acad. Sci. USA 81: 6851-6855). Typically the variable domains are obtained from an antibody from an experimental animal (the “parental antibody”), such as a rodent, and the constant domain sequences are obtained from human antibodies, so that the resulting chimeric antibody will be less likely to elicit an adverse immune response in a human subject than the parental (e.g. rodent) antibody.

As used herein, the term “humanized antibody” refers to forms of antibodies that contain sequences from both human and non-human (e.g., murine, rat) antibodies. In general, the humanized antibody will comprise substantially all of at least one, and typically two, variable domains, in which all or substantially all of the hypervariable loops correspond to those of a non-human immunoglobulin, and all or substantially all of the framework (FR) regions are those of a human immunoglobulin sequence. The humanized antibody may optionally comprise at least a portion of a human immunoglobulin constant region (Fc).

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” refers to an antibody that comprises mouse immunoglobulin sequences only. Alternatively, a fully human antibody may contain rat carbohydrate chains if produced in a rat, in a rat cell, or in a hybridoma derived from a rat cell. Similarly, “rat antibody” refers to an antibody that comprises rat immunoglobulin sequences only.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5l ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, the term “hypervariable region” refers to the amino acid residues of an antibody that are responsible for antigen-binding. The hypervariable region comprises amino acid residues from a “complementarity determining region” or “CDR” (i.e. residues 24-34 (CDRL1), 50-56 (CDRL2) and 89-97 (CDRL3) in the light chain variable domain and residues 31-35 (CDRH1), 50-65 (CDRH2) and 95-102 (CDRH3) in the heavy chain variable domain; Kabat et al. (1991) Sequences of Proteins of Immunological Interest, 5th Ed. Public Health Service, National Institutes of Health, Bethesda, Md.) and/or those residues from a “hypervariable loop” (i.e. residues 26-32 (CDRL1), 50-52 (CDRL2) and 91-96 (CDRL3) in the light chain variable domain and 26-32 (CDRH1), 53-55 (CDRH2) and 96-101 (CDRH3) in the heavy chain variable domain; Chothia and Lesk (1987) J. Mol. Biol. 196: 901-917). As used herein, the term “framework” or “FR” residues refers to those variable domain residues other than the hypervariable region residues defined herein as CDR residues. CDRs provide the majority of contact residues for the binding of the antibody to the antigen or epitope. CDRs of interest can be derived from donor antibody variable heavy and light chain sequences, and include analogs of the naturally occurring CDRs, which analogs also share or retain the same antigen binding specificity and/or neutralizing ability as the donor antibody from which they were derived.

Additionally, in some embodiments, the antibodies can take the form of a full length antibody, single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; a Microbody; a peptide aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; Affimers, a DuoBody, a Fv, a Fab, a Fab′, a F(ab′)2, a peptide mimetic molecule, or a synthetic molecule, as described in US Patent Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of each of which are hereby incorporated by reference in their entireties. See also, Storz MAbs. 2011 May-June; 3(3): 310-317, which is hereby incorporated by reference.

The term “antigen” as used herein means any molecule that has the ability to generate antibodies either directly or indirectly or that binds to antibody. Included within the definition of “antigen” is a protein-encoding nucleic acid. An “antigen” can also refer to the binding partner of an antibody. In some embodiments, the antigen is the IGF-1R protein expressed on the surface of a cell. In some embodiments, the cell is an intact cell. An intact cell is a cell that has not been lysed or broken open with the use of detergents or other reagents. A cell that has been treated with detergents or other reagents that breaks up the cellular membrane or punches holes in a cellular membrane is not an intact cell. For example, methods are provided herein for generating an antibody that binds to a IGF-1R protein, the method comprising culturing a cell comprising a nucleic acid molecule encoding the IGF-1R antibody.

As used herein, “specific binding” or “immunospecific binding” or “binds immunospecifically” refer to antibody binding to a predetermined antigen (e.g. IGF-1R) or epitope present on the antigen. In some embodiments, the antibody binds with a dissociation constant (K_{D}) of 10⁻⁷ M or less, and binds to the predetermined antigen with a K_{D} that is at least two-fold less than its K_{D} for binding to a non-specific antigen (e.g., BSA, casein, or another non-specific polypeptide) other than the predetermined antigen. The phrases “an antibody recognizing IGF-1R “and “an antibody specific for IGF-1R” are used interchangeably herein with the term “an antibody which binds immunospecifically to IGF-1R.” Reference in the present disclosure may be made to IGF-1R. The degree of specificity necessary for an anti-IGF-1R antibody may depend on the intended use of the antibody, and at any rate is defined by its suitability for use for an intended purpose. In some embodiments, the antibody, or binding compound derived from the antigen-binding site of an antibody, of the contemplated method binds to its antigen (IGF-1R), with an affinity that is at least two fold greater, at least ten times greater, at least 20-times greater, or at least 100-times greater than the affinity with any other antigen.

Methods for determining mAb specificity and affinity by competitive inhibition can be found in Harlow, et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc. and Wiley Interscience, N.Y., (1992, 1993), and Muller, Meth. Enzymol. 92:589 601 (1983), which references are entirely incorporated herein by reference.

The term “homolog” means protein sequences having between 40% and 100% sequence homology or identity to a reference sequence. Percent identity between two peptide chains can be determined by pair wise alignment using the default settings of the AlignX module of Vector NTI v.9.0.0 (Invitrogen Corp., Carslbad, Calif.). In some embodiments, the antibody, or antigenic binding fragment thereof has, at least 50, 60, 70, 80, 90, 91, 92, 93, 94, 95, 96, 97, 98, or 99% homology or identity to a sequence described herein. In some embodiments, the antibody has conservative substitutions as compared to a sequence described herein. Exemplary conservative substitutions are illustrated in Table 1 and are encompassed within the scope of the disclosed subject matter. The conservative substitution may reside in the framework regions, or in antigen-binding sites, as long they do not adversely affect the properties of the antibody. Substitutions may be made to improve antibody properties, for example stability or affinity. Conservative substitutions will produce molecules having functional and chemical characteristics similar to those molecules into which such modifications are made. Exemplary amino acid substitutions are shown in the table below.

TABLE
Exemplary Conservative Substitutions:

| Original Residue | Exemplary Conservative Substitutions |
|---|---|
| Ala | Val, Leu, Ile |
| Arg | Lys, Gln, Asn |
| Asn | Gln |
| Asp | Glu |
| Cys | Ser, Ala |
| Gln | Asn |
| Gly | Pro, Ala |
| His | Asn, Gln, Lys, Arg |
| Ile | Leu, Val, Met, Ala, Phe |
| Leu | Ile, Val, Met, Ala, Phe |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala |

In some embodiments, variants of the proteins and peptides provided herein are provided. In some embodiments, a variant comprises a substitution, deletions, or insertion. In some embodiments, the variant comprises 1, 2, 3, 4, 5, 6, 7, 8, 9, or 10 (e.g., 1-10) substitutions. As described herein, the substitutions can be conservative substitutions. In some embodiments, the substitution is non-conservative. In some embodiments, the variant comprises 1, 2, 3, 4, 5, 6, 7, 8, 9, or 10 (e.g., 1-10) deletions. In some embodiments, the variant comprises 1, 2, 3, 4, 5, 6, 7, 8, 9, or 10 (e.g., 1-10) insertions. In some embodiments, the substitutions, deletions, or insertions are present in the CDRs provided for herein. In some embodiments, the substitutions, deletions, or insertions are not present in the CDRs provided for herein.

The term “in combination with” as used herein means that the described agents can be administered to an animal or subject together in a mixture, concurrently as single agents or sequentially as single agents in any order.

The techniques to raise antibodies to small peptide sequences that recognize and bind to those sequences in the free or conjugated form or when presented as a native sequence in the context of a large protein are well known in the art. Such antibodies include murine, murine-human and human-human antibodies produced by hybridoma or recombinant techniques known in the art. Antibodies can also be produced in human, a mouse, sheep, a rat, a rabbit, a shark, a llama, or a chicken. In some embodiments, the antibody is produced in a chicken. The antibodies can also be produced in or other small animals.

The term “epitope” is meant to refer to that portion of any molecule capable of being recognized by and bound by an antibody at one or more of the Ab's antigen binding regions. Epitopes usually consist of chemically active surface groupings of molecules such as amino acids or sugar side chains and have specific three dimensional structural characteristics as well as specific charge characteristics. Example of epitopes include, but are not limited to, the residues described herein that form IGF-1R epitopes. In some embodiments, the epitope is only present in a non-denatured protein. In some embodiments, the epitope is only present in a denatured protein.

In some embodiments, the source for the DNA encoding a non-human antibody include cell lines which produce antibody, such as hybrid cell lines commonly known as hybridomas.

The hybrid cells are formed by the fusion of a non-human antibody-producing cell, typically a spleen cell of an animal immunized against either natural or recombinant antigen, or a peptide fragment of the antigen protein sequence. Alternatively, the non-human antibody-producing cell can be a B lymphocyte obtained from the blood, spleen, lymph nodes or other tissue of an animal immunized with the antigen.

The second fusion partner, which provides the immortalizing function, can be a lymphoblastoid cell or a plasmacytoma or myeloma cell, which is not itself an antibody producing cell, but is malignant. Fusion partner cells include, but are not limited to, the hybridoma SP2/0-Ag14, abbreviated as SP2/0 (ATCC CRL1581) and the myeloma P3X63Ag8 (ATCC TIB9), or its derivatives. See, e.g., Ausubel infra, Harlow infra, and Colligan infra, the contents of which references are incorporated entirely herein by reference.

The antibodies can be generated according the examples provided herein. Once the sequences are known, the antibodies can also be generated according to known methods. The antibodies can also be converted to different types, such as being converted to Human IgGs and the like. By converting the antibodies to a human antibody, a human subject should not identify the antibodies as foreign. The conversion of a non-human IgG antibody to a human IgG antibody is well known and can routinely be done once the native sequence is known. As discussed herein, the antibodies can be modified according to known methods. Such methods are described in, for example, Riechmann L, Clark M, Waldmann H, Winter G (1988). Reshaping human antibodies for therapy”. Nature 332 (6162): 332-323; Tsurushita N, Park M, Pakabunto K, Ong K, Avdalovic A, Fu H, Jia A, Visquez M, Kumar S. (2004). The antibody-producing cell contributing the nucleotide sequences encoding the antigen-binding region of the chimeric antibody can also be produced by transformation of a non-human, such as a primate, or a human cell. For example, a B lymphocyte which produces the antibody can be infected and transformed with a virus such as Epstein-Barr virus to yield an immortal antibody producing cell (Kozbor et al., Immunol. Today 4:72 79 (1983)). Alternatively, the B lymphocyte can be transformed by providing a transforming gene or transforming gene product, as is well-known in the art. See, e.g., Ausubel infra, Harlow infra, and Colligan infra, the contents of which references are incorporated entirely herein by reference. The cell fusions are accomplished by standard procedures well known to those skilled in the field of immunology. Fusion partner cell lines and methods for fusing and selecting hybridomas and screening for mAbs are well known in the art. See, e.g., Ausubel infra, Harlow infra, and Colligan infra, the contents of which references are incorporated entirely herein by reference.

In some embodiments, the antibody is a MAb which binds to IGF-1R. In some embodiments, the antibody binds to amino acids of an epitope of the IGF-1R.

In some embodiments, the antibody comprises a sequence as provided for herein.

The sequences of the antibodies can be modified to yield human IgG antibodies. The conversion of the sequences provided herein can be modified to yield other types of antibodies. The CDRs can also be linked to other antibodies, proteins, or molecules to create antibody fragments that bind to IGF-1R. This can be in the form of an antibody drug conjugate (“ADC”), a multi-specific molecule, or a chimeric antigen receptor. The CDRs and antibody sequences provided herein also be humanized or made fully human according to known methods. The sequences can also be made into chimeric antibodies as described herein.

In some embodiments, the antibody comprises an amino acid sequence comprising a sequence provided for herein or a fragment thereof. In some embodiments, the antibody comprises one or more amino acid sequences as provided herein, an antigen binding fragments, thereof, or a human IgG variant thereof. “A human IgG variant thereof” refers to an antibody that has been modified to be a human IgG when the starting antibody is not a human IgG antibody.

As described herein the production of antibodies with a known sequence is routine and can be done by any method. Accordingly, in some embodiments, a nucleic acid encoding an antibody or fragment thereof is provided. In some embodiments, the nucleic acid encodes a sequence provided for herein. The antibodies can also be modified to be chimeric antibodies or human antibodies. The antibodies can also be used in injectable pharmaceutical compositions. As also described herein, the antibodies can be isolated antibodies or engineered antibodies.

In some embodiments, “derivatives” of the antibodies, fragments, regions or derivatives thereof, which term includes those proteins encoded by truncated or modified genes to yield molecular species functionally resembling the immunoglobulin fragments are provided. The modifications include, but are not limited to, addition of genetic sequences coding for cytotoxic proteins such as plant and bacterial toxins. The modification can also include a reporter protein, such as a fluorescent or chemiluminescent tag. The fragments and derivatives can be produced in any manner.

The identification of these antigen binding region and/or epitopes recognized by Abs described herein provide the information necessary to generate additional monoclonal antibodies with similar binding characteristics and therapeutic or diagnostic utility that parallel the embodiments of this application.

The nucleic acid sequence encoding an antibody described herein can be genomic DNA or cDNA, or RNA (e.g. mRNA) which encodes at least one of the variable regions described herein. A convenient alternative to the use of chromosomal gene fragments as the source of DNA encoding the V region antigen-binding segment is the use of cDNA for the construction of chimeric immunoglobulin genes, e.g., as reported by Liu et al. (Proc. Natl. Acad. Sci., USA 84:3439 (1987) and J. Immunology 139:3521 (1987), which references are hereby entirely incorporated herein by reference. The use of cDNA requires that gene expression elements appropriate for the host cell be combined with the gene in order to achieve synthesis of the desired protein. The use of cDNA sequences is advantageous over genomic sequences (which contain introns), in that cDNA sequences can be expressed in bacteria or other hosts which lack appropriate RNA splicing systems.

For example, a cDNA encoding a V region antigen-binding segment able to detect, bind, to or neutralize a IGF-1R antigen can be provided using known methods based on the use of the amino acid sequences provided herein. Because the genetic code is degenerate, more than one codon can be used to encode a particular amino acid (Watson, et al., infra). Using the genetic code, one or more different oligonucleotides can be identified, each of which would be capable of encoding the amino acid. The probability that a particular oligonucleotide will, in fact, constitute the actual XXX-encoding sequence can be estimated by considering abnormal base pairing relationships and the frequency with which a particular codon is actually used (to encode a particular amino acid) in eukaryotic or prokaryotic cells expressing an antibody or fragment. Such “codon usage rules” are disclosed by Lathe, et al., J. Molec. Biol. 183:1 12 (1985). Using the “codon usage rules” of Lathe, a single oligonucleotide, or a set of oligonucleotides, that contains a theoretical “most probable” nucleotide sequence capable of encoding an antibody variable or constant region sequences is identified.

The variable regions described herein can be combined with any type of constant region including a human constant region or murine constant region. Human genes which encode the constant (C) regions of the antibodies, fragments and regions can be derived from a human fetal liver library, by known methods. Human C regions genes can be derived from any human cell including those which express and produce human immunoglobulins. The human C_{H} region can be derived from any of the known classes or isotypes of human H chains, including gamma, p, a, 6 or 8, and subtypes thereof, such as G1, G2, G3 and G4. Since the H chain isotype is responsible for the various effector functions of an antibody, the choice of C_{H} region will be guided by the desired effector functions, such as complement fixation, or activity in antibody-dependent cellular cytotoxicity (ADCC). Preferably, the C_{H} region is derived from gamma 1 (IgG1), gamma 3 (IgG3), gamma 4 (IgG4), or p (IgM). The human C_{L} region can be derived from either human L chain isotype, kappa or lambda. In some embodiments, the antibody comprises a Fc domain. In some embodiments, the Fc domain comprises a mutation to extend the half-life of the antibody. In some embodiments, the Fc domain comprises a mutation such as those described in U.S. Pat. No. 7,670,600, which is hereby incorporated by reference in its entirety. In some embodiment, the constant region comprises a mutation at position at amino acid residue 428 relative to a wild-type human IgG constant domain, numbered according to the EU numbering index of Kabat. Without being bound to any particular theory, an antibody comprising a mutation that corresponds to residue 428 can have an increased half-life compared to the half-life of an IgG having the wild-type human IgG constant domain. In some embodiments, the mutation is a substitution of the native residue with a threonine, leucine, phenylalanine or serine. In some embodiments, the antibody further comprises one or more amino acid substitutions relative to the corresponding wild-type human IgG constant domain at one or more of amino acid residues 251-256, 285-290, 308-314, 385-389, and 429-436, numbered according to the Kabat EU numbering index. The specific mutations or substitutions at these positions are described in U.S. Pat. No. 7,670,600, which is hereby incorporated by reference in its entirety.

Genes encoding human immunoglobulin C regions can be obtained from human cells by standard cloning techniques (Sambrook, et al. (Molecular Cloning: A Laboratory Manual, 2nd Edition, Cold Spring Harbor Press, Cold Spring Harbor, N.Y. (1989) and Ausubel et al., eds. Current Protocols in Molecular Biology (1987 1993)). Human C region genes are readily available from known clones containing genes representing the two classes of L chains, the five classes of H chains and subclasses thereof. Chimeric antibody fragments, such as F(ab′)₂ and Fab, can be prepared by designing a chimeric H chain gene which is appropriately truncated. For example, a chimeric gene encoding an H chain portion of an F(ab′)₂ fragment would include DNA sequences encoding the CH₁ domain and hinge region of the H chain, followed by a translational stop codon to yield the truncated molecule.

In some embodiments, the antibodies, murine, human, humanized, or chimeric antibodies, fragments and regions of the antibodies described herein are produced by cloning DNA segments encoding the H and L chain antigen-binding regions of a IGF-1R antigen specific antibody, and joining these DNA segments to DNA segments encoding C_{H} and C_{L} regions, respectively, to produce murine, human or chimeric immunoglobulin-encoding genes.

Thus, in some embodiments, a fused chimeric gene is created which comprises a first DNA segment that encodes at least the antigen-binding region of non-human origin, such as a functionally rearranged V region with joining (J) segment, linked to a second DNA segment encoding at least a part of a human C region.

Therefore, cDNA encoding the antibody V and C regions, the method of producing the antibody according to some of the embodiments described herein involve several steps, as exemplified below: 1. isolation of messenger RNA (mRNA) from the cell line producing an anti-IGF-1R antigen antibody and from optional additional antibodies supplying heavy and light constant regions; cloning and cDNA production therefrom; 2. preparation of a full length cDNA library from purified mRNA from which the appropriate V and/or C region gene segments of the L and H chain genes can be: (i) identified with appropriate probes, (ii) sequenced, and (iii) made compatible with a C or V gene segment from another antibody for a chimeric antibody; 3. Construction of complete H or L chain coding sequences by linkage of the cloned specific V region gene segments to cloned C region gene, as described above; 4. Expression and production of L and H chains in selected hosts, including prokaryotic and eukaryotic cells to provide murine-murine, human-murine, human-human or human murine antibodies.

Two coding DNA sequences are said to be “operably linked” if the linkage results in a continuously translatable sequence without alteration or interruption of the triplet reading frame. A DNA coding sequence is operably linked to a gene expression element if the linkage results in the proper function of that gene expression element to result in expression of the coding sequence.

As used herein and unless otherwise indicated, the term “about” is intended to mean±5% of the value it modifies. Thus, about 100 means 95 to 105.

In some embodiments, the antibodies described herein are used to detect the presence of the antigen. The present antibody can be used in any device or method to detect the presence of the antigen.

The term “purified” with referenced to an antibody refers to an antibody that is substantially free of other material that associates with the molecule in its natural environment. For instance, a purified protein is substantially free of the cellular material or other proteins from the cell or tissue from which it is derived. The term refers to preparations where the isolated protein is sufficiently pure to be analyzed, or at least 70% to 80% (w/w) pure, at least 80%-90% (w/w) pure, 90-95% pure; and, at least 95%, 96%, 97%, 98%, 99%, or 100% (w/w) pure. In some embodiments, the antibody is purified.

As an alternative to preparing monoclonal antibody-secreting hybridomas, a monoclonal antibody to a polypeptide may be identified and isolated by screening a recombinant combinatorial immunoglobulin library (e.g., an antibody phage display library) with a polypeptide described herein to thereby isolate immunoglobulin library members that bind to the polypeptide. Techniques and commercially available kits for generating and screening phage display libraries are well known to those skilled in the art. Additionally, examples of methods and reagents particularly amenable for use in generating and screening antibody or antigen binding protein display libraries can be found in the literature. Thus, the epitopes described herein can be used to screen for other antibodies that can be used therapeutically, diagnostically, or as research tools.

### Antibody Conjugates

The antibodies provided for herein may also be conjugated to a chemical moiety. The chemical moiety may be, inter alia, a polymer, a radionuclide or a cytotoxic factor. In some embodiments, this can be referred to as an antibody drug conjugate. In some embodiments, the chemical moiety is a polymer which increases the half-life of the antibody molecule in the body of a subject. Suitable polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)). Examples of chemical moieties include, but are not limited to, anti-mitotics, such as calicheamicins (e.g. ozogamicin), monomethyl auristatin E, mertansine, and the like. Other examples include, but are not limited to, biologically active anti-microtubule agents, alkylating agents and DNA minor groove binding agents. Other examples of are provided herein and below. The chemical moiety can be linked to the antibody through a linking group (maleimide), a cleaveble linker, such as a cathepsin cleavable linkers (valine-citrulline), and in some embodiments, one or more spacers (e.g. para-aminobenzylcarbamate). Without being bound to any particular theory, once the antibody conjugate binds IGF-1R it can be internalized and the chemical moiety can kill the cell or otherwise inhibit its growth. In some embodiments, the cell is a thyroid cell.

The antibodies and antibody fragments of the invention may also be conjugated with labels such as ⁹⁹Tc, ⁹⁰Y, ¹¹¹In ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰co, ⁵⁹Fe, ⁵⁷Se, ¹⁵²Eu, ⁶⁷Cu, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr and ⁵⁶Fe.

The antibodies and antibody fragments may also be conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The antibody molecules may also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, Momordica charantia inhibitor, curcin, crotin, Saponaria officinalis inhibitor, mitogellin, restrictocin, phenomycin, and enomycin.

Any method known in the art for conjugating the antibody molecules of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies are conventional and very well known in the art.

### Chimeric Antigen Receptors

The antibodies provided herein can also be incorporated into a chimeric antigen receptor (“CAR”) that can be used, for example, in a CAR-T cell. In some embodiments, the extracellular domain of the CAR can be an antibody as provided for herein. In some embodiments, the antibody is in a scFv format. CAR-T cells are a type of treatment in which a patient's T cells are modified so they will attack the cells that are expressing IGF-1R. T cells are taken from a patient's blood. Then the gene for a special receptor that binds to a certain protein on the patient's cells is added in the laboratory. In some embodiments, the receptor binds to IGF-1R using the binding regions of the antibodies provided for herein. The CAR-T cells comprising the IGF-1R antibody can then be used to treat a condition, such as those provided for herein.

In some embodiments, antibodies (e.g. an anti-IGF-1R antibody) are provided herein. In some embodiments, the antibody is a recombinant antibody that binds to a IGF-1R protein. In some embodiments, the IGF-1R protein is a human IGF-1R protein. In some embodiments, the IGF-1R protein that is recognized by the antibodies is in its native conformation (non-denatured) conformation. In some embodiments, the antibody does not specifically binds to a denatured IGF-1R protein. As used herein, the term “recombinant antibody” refers to an antibody that is not naturally occurring. In some embodiments, the term “recombinant antibody” refers to an antibody that is not isolated from a human subject.

In some embodiments, the antibody comprises a heavy and a light chain, wherein the heavy chain comprises a sequence of:

(SEQ ID NO: 371)
QVQLVQSGAEVVKPGASVKLSCKASGYTFTSYWMHWVKQRPGQGLEWIG
EINPSNGRTNYNQKFQGKATLTVDKSSSTAYMQLSSLTSEDSAVYYFAR
GRPDYYGSSKWYFDVWGQGTTVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHN
HYTQKSLSLSPGK;

and the light chain comprises a sequence of:

(SEQ ID NO: 374)
DVVMTQTPLSLPVSLGDPASISCRSSQSIVHSNVNTYLEWYLQKPGQSP
RLLIYKVSNRFSGVPDRFSGSGAGTDFTLRISRVEAEDLGIYYCFQGSH
VPPTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

In some embodiments, the heavy chain of SEQ ID NO: 371 comprises a C-terminal lysine residue that is added to the C-terminus of SEQ ID NO: 371.

In some embodiments, the antibody comprises a heavy and a light chain, wherein the heavy chain comprises a sequence of:

(SEQ ID NO: 372)
QVQLVQSGAEVVKPGASVKLSCKASGYTFTSYWMHWVKQRPGQGLEWIG
EINPSNGRTNYNQKFQGKATLTVDKSSSTAYMQLSSLTSEDSAVYYFAR
GRPDYYGSSKWYFDVWGQGTTVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGGP
SVFLFPPKPKDTLYITREPEVTCVVVDVSHEDPEVKFNWYVDGVEVHNA
KTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHN
HYTQKSLSLSPG

and the light chain comprises a sequence of SEQ ID NO: 374.

In some embodiments, the heavy chain of SEQ ID NO: 372 comprises a C-terminal lysine residue that is added to the C-terminus of SEQ ID NO: 372.

In some embodiments, the antibody comprises a heavy and a light chain, wherein the heavy chain comprises a sequence of:

(SEQ ID NO: 373)
QVQLVQSGAEVVKPGASVKLSSKASGYTFTSYWMHWVKQRPGQGLEWIG
EINPSNGRTNYNQKFQGKATLTVDKSSSTAYMQLSSLTSEDSAVYYFAR
GRPDYYGSSKWYFDVWGQGTTVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGGP
SVFLFPPKPKDTLYITREPEVTCVVVDVSHEDPEVKFNWYVDGVEVHNA
KTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHN
HYTQKSLSLSPG

and the light chain comprises a sequence of SEQ ID NO: 374.

In some embodiments, the heavy chain of SEQ ID NO: 373 comprises a C-terminal lysine residue that is added to the C-terminus of SEQ ID NO: 373.

In some embodiments, the antibody can comprise the heavy chain (HC) or light chain (LC) sequences, or combinations of HC and LC sequences, or variants thereof, as provided for in the table below:

| AB ID | LC and HC Sequence | LC Sequence | HC Sequence |
|---|---|---|---|
| VRDN-03100 | EIVLTQSPATLSLSPG | EIVLTQSPATLSLSPG | QVELVESGGGVVQPG |
| | ERATLSCRASQSVSSY | ERATLSCRASQSVSSY | RSQRLSCAASGFTFS |
| | LAWYQQKPGQAPRLLI | LAWYQQKPGQAPRLLI | SYGMHWVRQAPGKGL |
| | YDASKRATGIPARFSG | YDASKRATGIPARFSG | EWVAIIWFDGSSTYY |
| | SGSGTDFTLTISSLEP | SGSGTDFTLTISSLEP | ADSVRGRFTISRDNS |
| | EDFAVYYCQQRSKWPP | EDFAVYYCQQRSKWPP | KNTLYLQMNSLRAED |
| | WTFGQGTKVESKRIVA | WTFGQGTKVESKRTVA | TAVYFCARELGRRYF |
| | APSVFIFPPSDEQLKS | APSVFIFPPSDEQLKS | DLWGRGTLVSVSSAS |
| | GTASVVCLLNNFYPRE | GTASVVCLLNNFYPRE | TKGPSVFPLAPSSKS |
| | AKVQWKVDNALQSGNS | AKVQWKVDNALQSGNS | TSGGTAALGCLVKDY |
| | QESVTEQDSKDSTYSL | QESVTEQDSKDSTYSL | FPEPVTVSWNSGALT |
| | SSTLTLSKADYEKHKV | SSTLTLSKADYEKHKV | SGVHTFPAVLQSSGL |
| | YACEVTHQGLSSPVTK | YACEVTHQGLSSPVTK | YSLSSVVTVPSSSLG |
| | SFNRGEC | SFNRGEC | TQTYICNVNHKPSNT |
| | QVELVESGGGVVQPGR | (SEQ ID NO: 377) | KVDKKVEPKSCDKTH |
| | SQRLSCAASGFTESSY | | TCPPCPAPELLGGPS |
| | GMHWVRQAPGKGLEWV | | VFLFPPKPKDTLMIS |
| | AIIWEDGSSTYYADSV | | RTPEVTCVVVDVSHE |
| | RGRFTISRDNSKNTLY | | DPEVKFNWYVDGVEV |
| | LQMNSLRAEDTAVYFC | | HNAKTKPREEQYNST |
| | ARELGRRYFDLWGRGT | | YRVVSVLTVLHQDWL |
| | LVSVSSASTKGPSVFP | | NGKEYKCKVSNKALP |
| | LAPSSKSTSGGTAALG | | APIEKTISKAKGQPR |
| | CLVKDYFPEPVTVSWN | | EPQVYTLPPSRDELT |
| | SGALTSGVHTFPAVLQ | | KNQVSLTCLVKGFYP |
| | SSGLYSLSSVVTVPSS | | SDIAVEWESNGQPEN |
| | SLGTQTYICNVNHKPS | | NYKTTPPVLDSDGSF |
| | NTKVDKKVEPKSCDKT | | FLYSKLTVDKSRWQQ |
| | HTCPPCPAPELLGGPS | | GNVFSCSVMHEALHN |
| | VFLFPPKPKDTLMISR | | HYTQKSLSLSPGK |
| | TPEVTCVVVDVSHEDP | | (SEQ ID NO: |
| | EVKFNWYVDGVEVHNA | | 378) |
| | KTKPREEQYNSTYRVV | | |
| | SVLTVLHQDWLNGKEY | | |
| | KCKVSNKALPAPIEKT | | |
| | ISKAKGQPREPQVYTL | | |
| | PPSRDELTKNQVSLTC | | |
| | LVKGFYPSDIAVEWES | | |
| | NGQPENNYKTTPPVLD | | |
| | SDGSFFLYSKLTVDKS | | |
| | RWQQGNVFSCSVMHEA | | |
| | LHNHYTQKSLSLSPGK | | |
| | (SEQ ID NO: 379) | | |
| VRDN-02100 | DIVMTQSPLSLPVTPG | DIVMTQSPLSLPVTPG | QVQLQESGPGLVKPS |
| | EPASISCRSSQSIVHS | EPASISCRSSQSIVHS | ETLSLTCTVSGYSIT |
| | NGNTYLQWYLQKPGQS | NGNTYLQWYLQKPGQS | GGYLWNWIRQPPGKG |
| | PQLLIYKVSNRLYGVP | PQLLIYKVSNRLYGVP | LEWIGYISYDGTNNY |
| | DRFSGSGSGTDFTLKI | DRFSGSGSGTDFTLKI | KPSLKDRVTISRDTS |
| | SRVEAEDVGVYYCFQG | SRVEAEDVGVYYCFQG | KNQFSLKLSSVTAAD |
| | SHVPWTFGQGTKVEIK | SHVPWTFGQGTKVEIK | TAVYYCARYGRVFFD |
| | RTVAAPSVFIFPPSDE | RTVAAPSVFIFPPSDE | YWGQGTLVTVSSAST |
| | QLKSGTASVVCLLNNF | QLKSGTASVVCLLNNF | KGPSVFPLAPSSKST |
| | YPREAKVQWKVDNALQ | YPREAKVQWKVDNALQ | SGGTAALGCLVKDYF |
| | SGNSQESVTEQDSKDS | SGNSQESVTEQDSKDS | PEPVTVSWNSGALTS |
| | TYSLSSTLTLSKADYE | TYSLSSTLTLSKADYE | GVHTFPAVLQSSGLY |
| | KHKVYACEVTHQGLSS | KHKVYACEVTHQGLSS | SLSSVVTVPSSSLGT |
| | PVTKSFNRGEC | PVTKSFNRGEC | QTYICNVNHKPSNTK |
| | QVQLQESGPGLVKPSE | (SEQ ID NO: 380) | VDKRVEPKSCDKTHT |
| | TLSLTCTVSGYSITGG | | CPPCPAPELLGGPSV |
| | YLWNWIRQPPGKGLEW | | FLFPPKPKDTLMISR |
| | IGYISYDGTNNYKPSL | | TPEVTCVVVDVSHED |
| | KDRVTISRDTSKNQFS | | PEVKFNWYVDGVEVH |
| | LKLSSVTAADTAVYYC | | NAKTKPREEQYNSTY |
| | ARYGRVFFDYWGQGTL | | RVVSVLTVLHQDWLN |
| | VTVSSASTKGPSVFPL | | GKEYKCKVSNKALPA |
| | APSSKSTSGGTAALGC | | PIEKTISKAKGQPRE |
| | LVKDYFPEPVTVSWNS | | PQVYTLPPSREEMTK |
| | GALTSGVHTFPAVLQS | | NQVSLTCLVKGFYPS |
| | SGLYSLSSVVTVPSSS | | DIAVEWESNGQPENN |
| | LGTQTYICNVNHKPSN | | YKTTPPVLDSDGSFF |
| | TKVDKRVEPKSCDKTH | | LYSKLTVDKSRWQQG |
| | TCPPCPAPELLGGPSV | | NVFSCSVMHEALHNH |
| | FLFPPKPKDTLMISRT | | YTQKSLSLSPGK |
| | PEVTCVVVDVSHEDPE | | (SEQ ID NO: |
| | VKFNWYVDGVEVHNAK | | 381) |
| | TKPREEQYNSTYRVVS | | |
| | VLTVLHQDWLNGKEYK | | |
| | CKVSNKALPAPIEKTI | | |
| | SKAKGQPREPQVYTLP | | |
| | PSREEMTKNQVSLTCL | | |
| | VKGFYPSDIAVEWESN | | |
| | GQPENNYKTTPPVLDS | | |
| | DGSFFLYSKLTVDKSR | | |
| | WQQGNVFSCSVMHEAL | | |
| | HNHYTQKSLSLSPGK | | |
| | (SEQ ID NO: 382) | | |
| VRDN-02200 | SSELTQDPAVSVALGQ | SSELTQDPAVSVALGQ | EVQLVQSGAEVKKPG |
| | TVRITCQGDSLRSYYA | TVRITCQGDSLRSYYA | SSVKVSCKASGGTFS |
| | TWYQQKPGQAPILVIY | TWYQQKPGQAPILVIY | SYAISWVRQAPGQGL |
| | GENKRPSGIPDRFSGS | GENKRPSGIPDRFSGS | EWMGGIIPIFGTANY |
| | SSGNTASLTITGAQAE | SSGNTASLTITGAQAE | AQKFQGRVTITADKS |
| | DEADYYCKSRDGSGQH | DEADYYCKSRDGSGQH | TSTAYMELSSLRSED |
| | LVFGGGTKLTVLGQPK | LVFGGGTKLTVLGQPK | TAVYYCARAPLRFLE |
| | AAPSVTLFPPSSEELQ | AAPSVTLFPPSSEELQ | WSTQDHYYYYYMDVW |
| | ANKATLVCLISDFYPG | ANKATLVCLISDFYPG | GKGTTVTVSSASTKG |
| | AVTVAWKADSSPVKAG | AVTVAWKADSSPVKAG | PSVFPLAPSSKSTSG |
| | VETTTPSKQSNNKYAA | VETTTPSKQSNNKYAA | GTAALGCLVKDYFPE |
| | SSYLSLTPEQWKSHRS | SSYLSLTPEQWKSHRS | PVTVSWNSGALTSGV |
| | YSCQVTHEGSTVEKTV | YSCQVTHEGSTVEKTV | HTFPAVLQSSGLYSL |
| | APAECS | APAECS | SSVVTVPSSSLGTQT |
| | EVQLVQSGAEVKKPGS | (SEQ ID NO: 383) | YICNVNHKPSNTKVD |
| | SVKVSCKASGGTFSSY | | KKVEPKSCDKTHTCP |
| | AISWVRQAPGQGLEWM | | PCPAPELLGGPSVFL |
| | GGIIPIFGTANYAQKF | | FPPKPKDTLMISRTP |
| | QGRVTITADKSTSTAY | | EVTCVVVDVSHEDPE |
| | MELSSLRSEDTAVYYC | | VKFNWYVDGVEVHNA |
| | ARAPLRFLEWSTQDHY | | KTKPREEQYNSTYRV |
| | YYYYMDVWGKGTTVTV | | VSVLTVLHQDWLNGK |
| | SSASTKGPSVFPLAPS | | EYKCKVSNKALPAPI |
| | SKSTSGGTAALGCLVK | | EKTISKAKGQPREPQ |
| | DYFPEPVTVSWNSGAL | | VYTLPPSREEMTKNQ |
| | TSGVHTFPAVLQSSGL | | VSLTCLVKGFYPSDI |
| | YSLSSVVTVPSSSLGT | | AVEWESNGQPENNYK |
| | QTYICNVNHKPSNTKV | | TTPPVLDSDGSFFLY |
| | DKKVEPKSCDKTHTCP | | SKLTVDKSRWQQGNV |
| | PCPAPELLGGPSVFLF | | FSCSVMHEALHNHYT |
| | PPKPKDTLMISRTPEV | | QKSLSLSPGK |
| | TCVVVDVSHEDPEVKF | | (SEQ ID NO: |
| | NWYVDGVEVHNAKTKP | | 384) |
| | REEQYNSTYRVVSVLT | | |
| | VLHQDWLNGKEYKCKV | | |
| | SNKALPAPIEKTISKA | | |
| | KGQPREPQVYTLPPSR | | |
| | EEMTKNQVSLTCLVKG | | |
| | FYPSDIAVEWESNGQP | | |
| | ENNYKTTPPVLDSDGS | | |
| | FFLYSKLTVDKSRWQQ | | |
| | GNVFSCSVMHEALHNH | | |
| | YTQKSLSLSPGK | | |
| | (SEQ ID NO: 385) | | |
| VRDN-02300 | DIQMTQFPSSLSASVG | DIQMTQFPSSLSASVG | EVQLLESGGGLVQPG |
| | DRVTITCRASQGIRND | DRVTITCRASQGIRND | GSLRLSCTASGFTFS |
| | LGWYQQKPGKAPKRLI | LGWYQQKPGKAPKRLI | SYAMNWVRQAPGKGL |
| | YAASRLHRGVPSRFSG | YAASRLHRGVPSRFSG | EWVSAISGSGGTTFY |
| | SGSGTEFTLTISSLQP | SGSGTEFTLTISSLQP | ADSVKGRFTISRDNS |
| | EDFATYYCLQHNSYPC | EDFATYYCLQHNSYPC | RTTLYLQMNSLRAED |
| | SFGQGTKLEIKRTVAA | SFGQGTKLEIKRTVAA | TAVYYCAKDLGWSDS |
| | PSVFIFPPSDEQLKSG | PSVFIFPPSDEQLKSG | YYYYYGMDVWGQGTT |
| | TASVVCLLNNFYPREA | TASVVCLLNNFYPREA | VTVSSASTKGPSVFP |
| | KVQWKVDNALQSGNSQ | KVQWKVDNALQSGNSQ | LAPCSRSTSESTAAL |
| | ESVTEQDSKDSTYSLS | ESVTEQDSKDSTYSLS | GCLVKDYFPEPVTVS |
| | STLTLSKADYEKHKVY | STLTLSKADYEKHKVY | WNSGALTSGVHTFPA |
| | ACEVTHQGLSSPVTKS | ACEVTHQGLSSPVTKS | VLQSSGLYSLSSVVT |
| | FNRGEC | FNRGEC | VPSSNFGTQTYTCNV |
| | EVQLLESGGGLVQPGG | (SEQ ID NO: 386) | DHKPSNTKVDKTVER |
| | SLRLSCTASGFTFSSY | | KCCVECPPCPAPPVA |
| | AMNWVRQAPGKGLEWV | | GPSVFLFPPKPKDTL |
| | SAISGSGGTTFYADSV | | MISRTPEVTCVVVDV |
| | KGRFTISRDNSRTTLY | | SHEDPEVQFNWYVDG |
| | LQMNSLRAEDTAVYYC | | VEVHNAKTKPREEQF |
| | AKDLGWSDSYYYYYGM | | NSTFRVVSVLTVVHQ |
| | DVWGQGTTVTVSSAST | | DWLNGKEYKCKVSNK |
| | KGPSVFPLAPCSRSTS | | GLPAPIEKTISKTKG |
| | ESTAALGCLVKDYFPE | | QPREPQVYTLPPSRE |
| | PVTVSWNSGALTSGVH | | EMTKNQVSLTCLVKG |
| | TFPAVLQSSGLYSLSS | | FYPSDIAVEWESNGQ |
| | VVTVPSSNFGTQTYTC | | PENNYKTTPPMLDSD |
| | NVDHKPSNTKVDKTVE | | GSFFLYSKLTVDKSR |
| | RKCCVECPPCPAPPVA | | WQQGNVFSCSVMHEA |
| | GPSVFLFPPKPKDTLM | | LHNHYTQKSLSLSPG |
| | ISRTPEVTCVVVDVSH | | (SEQ ID NO: |
| | EDPEVQFNWYVDGVEV | | 387) |
| | HNAKTKPREEQFNSTF | | |
| | RVVSVLTVVHQDWLNG | | |
| | KEYKCKVSNKGLPAPI | | |
| | EKTISKTKGQPREPQV | | |
| | YTLPPSREEMTKNQVS | | |
| | LTCLVKGFYPSDIAVE | | |
| | WESNGQPENNYKTTPP | | |
| | MLDSDGSFFLYSKLTV | | |
| | DKSRWQQGNVFSCSVM | | |
| | HEALHNHYTQKSLSLS | | |
| | PG (SEQ ID NO: | | |
| | 388) | | |
| VRDN-02400 | DVVMTQSPLSLPVTPG | DVVMTQSPLSLPVTPG | QVQLQESGPGLVKPS |
| | EPASISCRSSQSLLHS | EPASISCRSSQSLLHS | GTLSLTCAVSGGSIS |
| | NGYNYLDWYLQKPGQS | NGYNYLDWYLQKPGQS | SSNWWSWVRQPPGKG |
| | PQLLIYLGSNRASGVP | PQLLIYLGSNRASGVP | LEWIGEIYHSGSTNY |
| | DRFSGSGSGTDFTLKI | DRFSGSGSGTDFTLKI | NPSLKSRVTISVDKS |
| | SRVEAEDVGVYYCMQG | SRVEAEDVGVYYCMQG | KNQFSLKLSSVTAAD |
| | THWPLTFGQGTKVEIK | THWPLTFGQGTKVEIK | TAVYYCARWTGRTDA |
| | RTVAAPSVFIFPPSDE | RTVAAPSVFIFPPSDE | FDIWGQGTMVTVSSA |
| | QLKSGTASVVCLLNNF | QLKSGTASVVCLLNNF | STKGPSVFPLAPSSK |
| | YPREAKVQWKVDNALQ | YPREAKVQWKVDNALQ | STSGGTAALGCLVKD |
| | SGNSQESVTEQDSKDS | SGNSQESVTEQDSKDS | YFPEPVTVSWNSGAL |
| | TYSLSSTLTLSKADYE | TYSLSSTLTLSKADYE | TSGVHTFPAVLQSSG |
| | KHKVYACEVTHQGLSS | KHKVYACEVTHQGLSS | LYSLSSVVTVPSSSL |
| | PVTKSFNRGEC | PVTKSFNRGEC | GTQTYICNVNHKPSN |
| | QVQLQESGPGLVKPSG | (SEQ ID NO: 389) | TKVDKKVEPKSCDKT |
| | TLSLTCAVSGGSISSS | | HTCPPCPAPELLGGP |
| | NWWSWVRQPPGKGLEW | | SVFLFPPKPKDTLMI |
| | IGEIYHSGSTNYNPSL | | SRTPEVTCVVVDVSH |
| | KSRVTISVDKSKNQFS | | EDPEVKFNWYVDGVE |
| | LKLSSVTAADTAVYYC | | VHNAKTKPREEQYNS |
| | ARWTGRTDAFDIWGQG | | TYRVVSVLTVLHQDW |
| | TMVTVSSASTKGPSVE | | LNGKEYKCKVSNKAL |
| | PLAPSSKSTSGGTAAL | | PAPIEKTISKAKGQP |
| | GCLVKDYFPEPVTVSW | | REPQVYTLPPSRDEL |
| | NSGALTSGVHTFPAVL | | TKNQVSLTCLVKGFY |
| | QSSGLYSLSSVVTVPS | | PSDIAVEWESNGQPE |
| | SSLGTQTYICNVNHKP | | NNYKTTPPVLDSDGS |
| | SNTKVDKKVEPKSCDK | | FFLYSKLTVDKSRWQ |
| | THTCPPCPAPELLGGP | | QGNVFSCSVMHEALH |
| | SVFLFPPKPKDTLMIS | | NHYTQKSLSLSPGK |
| | RTPEVTCVVVDVSHED | | (SEQ ID NO: |
| | PEVKFNWYVDGVEVHN | | 390) |
| | AKTKPREEQYNSTYRV | | |
| | VSVLTVLHQDWLNGKE | | |
| | YKCKVSNKALPAPIEK | | |
| | TISKAKGQPREPQVYT | | |
| | LPPSRDELTKNQVSLT | | |
| | CLVKGFYPSDIAVEWE | | |
| | SNGQPENNYKTTPPVL | | |
| | DSDGSFFLYSKLTVDK | | |
| | SRWQQGNVFSCSVMHE | | |
| | ALHNHYTQKSLSLSPG | | |
| | K (SEQ ID NO: | | |
| | 391) | | |
| VRDN-02500 | EIVLTQSPGTLSVSPG | EIVLTQSPGTLSVSPG | EVQLVQSGGGLVKPG |
| | ERATLSCRASQSIGSS | ERATLSCRASQSIGSS | GSLRLSCAASGFTFS |
| | LHWYQQKPGQAPRLLI | LHWYQQKPGQAPRLLI | SFAMHWVRQAPGKGL |
| | KYASQSLSGIPDRFSG | KYASQSLSGIPDRFSG | EWISVIDTRGATYYA |
| | SGSGTDFTLTISRLEP | SGSGTDETLTISRLEP | DSVKGRFTISRDNAK |
| | EDFAVYYCHQSSRLPH | EDFAVYYCHQSSRLPH | NSLYLQMNSLRAEDT |
| | TFGQGTKVEIKRTVAA | TFGQGTKVEIKRTVAA | AVYYCARLGNFYYGM |
| | PSVFIFPPSDEQLKSG | PSVFIFPPSDEQLKSG | DVWGQGTTVTVSSAS |
| | TASVVCLLNNFYPREA | TASVVCLLNNFYPREA | TKGPSVFPLAPSSKS |
| | KVQWKVDNALQSGNSQ | KVQWKVDNALQSGNSQ | TSGGTAALGCLVKDY |
| | ESVTEQDSKDSTYSLS | ESVTEQDSKDSTYSLS | FPEPVTVSWNSGALT |
| | STLTLSKADYEKHKVY | STLTLSKADYEKHKVY | SGVHTFPAVLQSSGL |
| | ACEVTHQGLSSPVTKS | ACEVTHQGLSSPVTKS | YSLSSVVTVPSSSLG |
| | FNRGEC | FNRGEC | TQTYICNVNHKPSNT |
| | EVQLVQSGGGLVKPGG | (SEQ ID NO: 392) | KVDKKVEPKSCDKTH |
| | SLRLSCAASGFTFSSF | | TCPPCPAPELLGGPS |
| | AMHWVRQAPGKGLEWI | | VFLFPPKPKDTLMIS |
| | SVIDTRGATYYADSVK | | RTPEVTCVVVDVSHE |
| | GRFTISRDNAKNSLYL | | DPEVKFNWYVDGVEV |
| | QMNSLRAEDTAVYYCA | | HNAKTKPREEQYNST |
| | RLGNFYYGMDVWGQGT | | YRVVSVLTVLHQDWL |
| | TVTVSSASTKGPSVFP | | NGKEYKCKVSNKALP |
| | LAPSSKSTSGGTAALG | | APIEKTISKAKGQPR |
| | CLVKDYFPEPVTVSWN | | EPQVYTLPPSRDELT |
| | SGALTSGVHTFPAVLQ | | KNQVSLTCLVKGFYP |
| | SSGLYSLSSVVTVPSS | | SDIAVEWESNGQPEN |
| | SLGTQTYICNVNHKPS | | NYKTTPPVLDSDGSF |
| | NTKVDKKVEPKSCDKT | | FLYSKLTVDKSRWQQ |
| | HTCPPCPAPELLGGPS | | GNVFSCSVMHEALHN |
| | VFLFPPKPKDTLMISR | | HYTQKSLSLSPGK |
| | TPEVTCVVVDVSHEDP | | (SEQ ID NO: |
| | EVKFNWYVDGVEVHNA | | 393) |
| | KTKPREEQYNSTYRVV | | |
| | SVLTVLHQDWLNGKEY | | |
| | KCKVSNKALPAPIEKT | | |
| | ISKAKGQPREPQVYTL | | |
| | PPSRDELTKNQVSLTC | | |
| | LVKGFYPSDIAVEWES | | |
| | NGQPENNYKTTPPVLD | | |
| | SDGSFFLYSKLTVDKS | | |
| | RWQQGNVFSCSVMHEA | | |
| | LHNHYTQKSLSLSPGK | | |
| | (SEQ ID NO: 394) | | |
| VRDN-02700 | DIVMTQSPLSLPVTPG | DIVMTQSPLSLPVTPG | QVQLQESGPGLVKPS |
| | EPASISCRSSQSIVHS | EPASISCRSSQSIVHS | ETLSLTCTVSGYSIT |
| | NGNTYLQWYLQKPGQS | NGNTYLQWYLQKPGQS | GGYLWNWIRQPPGKG |
| | PQLLIYKVSNRLYGVP | PQLLIYKVSNRLYGVP | LEWIGYISYDGINNY |
| | DRFSGSGSGTDFTLKI | DRFSGSGSGTDFTLKI | KPSLKDRVTISRDTS |
| | SRVEAEDVGVYYCFQG | SRVEAEDVGVYYCFQG | KNQFSLKLSSVTAAD |
| | SHVPWTFGQGTKVEIK | SHVPWTFGQGTKVEIK | TAVYYCARYGRVFFD |
| | RTVAAPSVFIFPPSDE | RTVAAPSVFIFPPSDE | YWGQGTLVTVSSAST |
| | QLKSGTASVVCLLNNF | QLKSGTASVVCLLNNF | KGPSVFPLAPSSKST |
| | YPREAKVQWKVDNALQ | YPREAKVQWKVDNALQ | SGGTAALGCLVKDYF |
| | SGNSQESVTEQDSKDS | SGNSQESVTEQDSKDS | PEPVTVSWNSGALTS |
| | TYSLSSTLTLSKADYE | TYSLSSTLTLSKADYE | GVHTFPAVLQSSGLY |
| | KHKVYACEVTHQGLSS | KHKVYACEVTHQGLSS | SLSSVVTVPSSSLGT |
| | PVTKSFNRGEC | PVTKSFNRGEC (SEQ | QTYICNVNHKPSNTK |
| | QVQLQESGPGLVKPSE | ID NO: 380) | VDKRVEPKSCDKTHT |
| | TLSLTCTVSGYSITGG | | CPPCPAPELLGGPSV |
| | YLWNWIRQPPGKGLEW | | FLFPPKPKDTLYITR |
| | IGYISYDGTNNYKPSL | | EPEVTCVVVDVSHED |
| | KDRVTISRDTSKNQFS | | PEVKFNWYVDGVEVH |
| | LKLSSVTAADTAVYYC | | NAKTKPREEQYNSTY |
| | ARYGRVFFDYWGQGTL | | RVVSVLTVLHQDWLN |
| | VTVSSASTKGPSVEPL | | GKEYKCKVSNKALPA |
| | APSSKSTSGGTAALGC | | PIEKTISKAKGQPRE |
| | LVKDYFPEPVTVSWNS | | PQVYTLPPSREEMTK |
| | GALTSGVHTFPAVLQS | | NQVSLTCLVKGFYPS |
| | SGLYSLSSVVTVPSSS | | DIAVEWESNGQPENN |
| | LGTQTYICNVNHKPSN | | YKTTPPVLDSDGSFF |
| | TKVDKRVEPKSCDKTH | | LYSKLTVDKSRWQQG |
| | TCPPCPAPELLGGPSV | | NVFSCSVMHEALHNH |
| | FLFPPKPKDTLYITRE | | YTQKSLSLSPG |
| | PEVTCVVVDVSHEDPE | | (SEQ ID NO: |
| | VKFNWYVDGVEVHNAK | | 395) |
| | TKPREEQYNSTYRVVS | | |
| | VLTVLHQDWLNGKEYK | | |
| | CKVSNKALPAPIEKTI | | |
| | SKAKGQPREPQVYTLP | | |
| | PSREEMTKNQVSLTCL | | |
| | VKGFYPSDIAVEWESN | | |
| | GQPENNYKTTPPVLDS | | |
| | DGSFFLYSKLTVDKSR | | |
| | WQQGNVFSCSVMHEAL | | |
| | HNHYTQKSLSLSPG | | |
| | (SEQ ID NO: 396) | | |

In some embodiments, the antibody can comprise the heavy variable region (VH) or light variable region (VL) sequence, or the combination of VH and VL sequences, or variants, as provided for in the table below:

| AB ID | VH and VL Sequence | VH Sequence | VL Sequence |
|---|---|---|---|
| VRDN-03100 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 1) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 123) | | |
| VRDN-03001 | QVQLVESGGGVVQPGR | QVQLVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 3) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 124) | | |
| VRDN-03002 | QVELVESGGGVVQPGR | QVELVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 4) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 125) | | |
| VRDN-03003 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 5) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 126) | | |
| VRDN-03004 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVTVSS | LVTVSS | (SEQ ID NO: 2) |
| | EIVLTQSPATLSLSPG | (SEQ ID NO: 6) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 127) | | |
| VRDN-03005 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVESK |
| | LVTVSS | LVTVSS | (SEQ ID NO: 2) |
| | EIVLTQSPATLSLSPG | (SEQ ID NO: 7) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 128) | | |
| VRDN-03006 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVEIK |
| | LVSVSS | LVSVSS | (SEQ ID NO: 8) |
| | EIVLTQSPATLSLSPG | (SEQ ID NO: 1) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 129) | | |
| VRDN-03007 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 7) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 130) | | |
| VRDN-03008 | QVELVESGGGVVQPGR | QVELVESGGGVVQPGR | EIVLTQSPATLSLSPG |
| | SQRLSCAASGFTFSHY | SQRLSCAASGFTFSHY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 9) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 131) | | |
| VRDN-03009 | QVELVESGGGVVQPGR | QVELVESGGGVVQPGR | EIVLTQSPATLSLSPG |
| | SQRLSCAASGFTFSSH | SQRLSCAASGFTFSSH | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 10) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 132) | | |
| VRDN-03010 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | HMHWVRQAPGKGLEWV | HMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 11) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 133) | | |
| VRDN-03011 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GHHWVRQAPGKGLEWV | GHHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 12) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 134) | | |
| VRDN-03012 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AHIWFDGSSTYYADSV | AHIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 13) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 135) | | |
| VRDN-03013 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIHWFDGSSTYYADSV | AIHWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 14) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 136) | | |
| VRDN-03014 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIHFDGSSTYYADSV | AIIHFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 15) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 137) | | |
| VRDN-03015 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWHDGSSTYYADSV | AIIWHDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 16) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 138) | | |
| VRDN-03016 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFHGSSTYYADSV | AIIWFHGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 17) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 139) | | |
| VRDN-03017 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDHSSTYYADSV | AIIWFDHSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 18) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 140) | | |
| VRDN-03018 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGHSTYYADSV | AIIWFDGHSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 19) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 141) | | |
| VRDN-03019 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSHTYYADSV | AIIWFDGSHTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 20) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 142) | | |
| VRDN-03020 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSHYYADSV | AIIWFDGSSHYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 21) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 143) | | |
| VRDN-03021 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTHYADSV | AIIWFDGSSTHYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 22) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 144) | | |
| VRDN-03022 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYHADSV | AIIWFDGSSTYHADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 23) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 145) | | |
| VRDN-03023 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYHDSV | AIIWFDGSSTYYHDSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 24) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 146) | | |
| VRDN-03024 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYAHSV | AIIWFDGSSTYYAHSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 25) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 147) | | |
| VRDN-03025 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADHV | AIIWFDGSSTYYADHV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 26) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 148) | | |
| VRDN-03026 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSH | AIIWFDGSSTYYADSH | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 27) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 149) | | |
| VRDN-03027 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | HGRFTISRDNSKNTLY | HGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 28) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 150) | | |
| VRDN-03028 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RHRFTISRDNSKNTLY | RHRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 2) |
| | EIVLTQSPATLSLSPG | (SEQ ID NO: 29) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 151) | | |
| VRDN-03029 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARHLGRRYFDLWGRGT | ARHLGRRYFDLWGRGT | WTFGQGTKVESK |
| | LVSVSS | LVSVSS | (SEQ ID NO: 2) |
| | EIVLTQSPATLSLSPG | (SEQ ID NO: 30) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 152) | | |
| VRDN-03030 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | AREHGRRYFDLWGRGT | AREHGRRYFDLWGRGT | WTFGQGTKVESK |
| | LVSVSS | LVSVSS | (SEQ ID NO: 2) |
| | EIVLTQSPATLSLSPG | (SEQ ID NO: 31) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 153) | | |
| VRDN-03031 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELHRRYFDLWGRGT | ARELHRRYFDLWGRGT | WTFGQGTKVESK |
| | LVSVSS | LVSVSS | (SEQ ID NO: 2) |
| | EIVLTQSPATLSLSPG | (SEQ ID NO: 32) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 154) | | |
| VRDN-03032 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGHRYFDLWGRGT | ARELGHRYFDLWGRGT | WTFGQGTKVESK |
| | LVSVSS | LVSVSS | (SEQ ID NO: 2) |
| | EIVLTQSPATLSLSPG | (SEQ ID NO: 33) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 155) | | |
| VRDN-03033 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRHYFDLWGRGT | ARELGRHYFDLWGRGT | WTFGQGTKVESK |
| | LVSVSS | LVSVSS | (SEQ ID NO: 2) |
| | EIVLTQSPATLSLSPG | (SEQ ID NO: 34) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 156) | | |
| VRDN-03034 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRHFDLWGRGT | ARELGRRHFDLWGRGT | WTFGQGTKVESK |
| | LVSVSS | LVSVSS | (SEQ ID NO: 2) |
| | EIVLTQSPATLSLSPG | (SEQ ID NO: 35) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 157) | | |
| VRDN-03035 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYHDLWGRGT | ARELGRRYHDLWGRGT | WTFGQGTKVESK |
| | LVSVSS | LVSVSS | (SEQ ID NO: 2) |
| | EIVLTQSPATLSLSPG | (SEQ ID NO: 36) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 158) | | |
| VRDN-03036 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFHLWGRGT | ARELGRRYFHLWGRGT | WTFGQGTKVESK |
| | LVSVSS | LVSVSS | (SEQ ID NO: 2) |
| | EIVLTQSPATLSLSPG | (SEQ ID NO: 37) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 159) | | |
| VRDN-03037 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDHWGRGT | ARELGRRYFDHWGRGT | WTFGQGTKVESK |
| | LVSVSS | LVSVSS | (SEQ ID NO: 2) |
| | EIVLTQSPATLSLSPG | (SEQ ID NO: 38) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 160) | | |
| VRDN-03038 | QVELVESGGGVVQPGR | QVELVESGGGVVQPGR | EIVLTQSPATLSLSPG |
| | SQRLSCAASGFTFSSY | SQRLSCAASGFTFSSY | ERATLSCHASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 39) |
| | EIVLTQSPATLSLSPG | (SEQ ID NO: 1) | |
| | ERATLSCHASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 161) | | |
| VRDN-03039 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRHSQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 40) |
| | EIVLTQSPATLSLSPG | (SEQ ID NO: 1) | |
| | ERATLSCRHSQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 162) | | |
| VRDN-03040 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRAHQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 41) |
| | EIVLTQSPATLSLSPG | (SEQ ID NO: 1) | |
| | ERATLSCRAHQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 163) | | |
| VRDN-03041 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASHSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 42) |
| | EIVLTQSPATLSLSPG | (SEQ ID NO: 1) | |
| | ERATLSCRASHSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 164) | | |
| VRDN-03042 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQHVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 43) |
| | EIVLTQSPATLSLSPG | (SEQ ID NO: 1) | |
| | ERATLSCRASQHVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 165) | | |
| VRDN-03043 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSHSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 44) |
| | EIVLTQSPATLSLSPG | (SEQ ID NO: 1) | |
| | ERATLSCRASQSHSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 166) | | |
| VRDN-03044 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVHSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 45) |
| | EIVLTQSPATLSLSPG | (SEQ ID NO: 1) | |
| | ERATLSCRASQSVHSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 167) | | |
| VRDN-03045 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSHY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 46) |
| | EIVLTQSPATLSLSPG | (SEQ ID NO: 1) | |
| | ERATLSCRASQSVSHY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 168) | | |
| VRDN-03046 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSH |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 47) |
| | EIVLTQSPATLSLSPG | (SEQ ID NO: 1) | |
| | ERATLSCRASQSVSSH | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 169) | | |
| VRDN-03047 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | HAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 48) |
| | EIVLTQSPATLSLSPG | (SEQ ID NO: 1) | |
| | ERATLSCRASQSVSSY | | |
| | HAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 170) | | |
| VRDN-03048 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LHWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 49) |
| | EIVLTQSPATLSLSPG | (SEQ ID NO: 1) | |
| | ERATLSCRASQSVSSY | | |
| | LHWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVESK | | |
| | (SEQ ID NO: 171) | | |
| VRDN-03049 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YHASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 50) |
| | EIVLTQSPATLSLSPG | (SEQ ID NO: 1) | |
| | ERATLSCRASQSVSSY | | |
| | LAWYQQKPGQAPRLLI | | |
| | YHASKRATGIPARFSG | | |
| | SGSGTDFTLTISSLEP | | |
| | EDFAVYYCQQRSKWPP | | |
| | WTFGQGTKVESK | | |
| | (SEQ ID NO: 172) | | |
| VRDN-03050 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDHSKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 51) |
| | EIVLTQSPATLSLSPG | (SEQ ID NO: 1) | |
| | ERATLSCRASQSVSSY | | |
| | LAWYQQKPGQAPRLLI | | |
| | YDHSKRATGIPARFSG | | |
| | SGSGTDFTLTISSLEP | | |
| | EDFAVYYCQQRSKWPP | | |
| | WTFGQGTKVESK | | |
| | (SEQ ID NO: 173) | | |
| VRDN-03051 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDAHKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 52) |
| | EIVLTQSPATLSLSPG | (SEQ ID NO: 1) | |
| | ERATLSCRASQSVSSY | | |
| | LAWYQQKPGQAPRLLI | | |
| | YDAHKRATGIPARFSG | | |
| | SGSGTDFTLTISSLEP | | |
| | EDFAVYYCQQRSKWPP | | |
| | WTFGQGTKVESK | | |
| | (SEQ ID NO: 174) | | |
| VRDN-03052 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASHRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 53) |
| | EIVLTQSPATLSLSPG | (SEQ ID NO: 1) | |
| | ERATLSCRASQSVSSY | | |
| | LAWYQQKPGQAPRLLI | | |
| | YDASHRATGIPARFSG | | |
| | SGSGTDFTLTISSLEP | | |
| | EDFAVYYCQQRSKWPP | | |
| | WTFGQGTKVESK | | |
| | (SEQ ID NO: 175) | | |
| VRDN-03053 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKHATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 54) |
| | EIVLTQSPATLSLSPG | (SEQ ID NO: 1) | |
| | ERATLSCRASQSVSSY | | |
| | LAWYQQKPGQAPRLLI | | |
| | YDASKHATGIPARFSG | | |
| | SGSGTDFTLTISSLEP | | |
| | EDFAVYYCQQRSKWPP | | |
| | WTFGQGTKVESK | | |
| | (SEQ ID NO: 176) | | |
| VRDN-03054 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRHTGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 55) |
| | EIVLTQSPATLSLSPG | (SEQ ID NO: 1) | |
| | ERATLSCRASQSVSSY | | |
| | LAWYQQKPGQAPRLLI | | |
| | YDASKRHTGIPARFSG | | |
| | SGSGTDFTLTISSLEP | | |
| | EDFAVYYCQQRSKWPP | | |
| | WTFGQGTKVESK | | |
| | (SEQ ID NO: 177) | | |
| VRDN-03055 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRAHGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WTFGQGTKVESK |
| | LVSVSS | LVSVSS | (SEQ ID NO: 56) |
| | EIVLTQSPATLSLSPG | (SEQ ID NO: 1) | |
| | ERATLSCRASQSVSSY | | |
| | LAWYQQKPGQAPRLLI | | |
| | YDASKRAHGIPARFSG | | |
| | SGSGTDFTLTISSLEP | | |
| | EDFAVYYCQQRSKWPP | | |
| | WTFGQGTKVESK | | |
| | (SEQ ID NO: 178) | | |
| VRDN-03056 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCHQRSKWPP |
| | ARELGRRYFDLWGRGT | ARELGRRYFDLWGRGT | WTFGQGTKVESK |
| | LVSVSS | LVSVSS | (SEQ ID NO: 57) |
| | EIVLTQSPATLSLSPG | (SEQ ID NO: 1) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCHQRSKWPP | | |
| | WTFGQGTKVESK | | |
| | (SEQ ID NO: 179) | | |
| VRDN-03057 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQHRSKWPP |
| | ARELGRRYFDLWGRGT | ARELGRRYFDLWGRGT | WTFGQGTKVESK |
| | LVSVSS | LVSVSS | (SEQ ID NO: 58) |
| | EIVLTQSPATLSLSPG | (SEQ ID NO: 1) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQHRSKWPP | | |
| | WTFGQGTKVESK | | |
| | (SEQ ID NO: 180) | | |
| VRDN-03058 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQHSKWPP |
| | ARELGRRYFDLWGRGT | ARELGRRYFDLWGRGT | WTFGQGTKVESK |
| | LVSVSS | LVSVSS | (SEQ ID NO: 59) |
| | EIVLTQSPATLSLSPG | (SEQ ID NO: 1) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQHSKWPP | | |
| | WTFGQGTKVESK | | |
| | (SEQ ID NO: 181) | | |
| VRDN-03059 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRHKWPP |
| | ARELGRRYFDLWGRGT | ARELGRRYFDLWGRGT | WTFGQGTKVESK |
| | LVSVSS | LVSVSS | (SEQ ID NO: 60) |
| | EIVLTQSPATLSLSPG | (SEQ ID NO: 1) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRHKWPP | | |
| | WTFGQGTKVESK | | |
| | (SEQ ID NO: 182) | | |
| VRDN-03060 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSHWPP |
| | ARELGRRYFDLWGRGT | ARELGRRYFDLWGRGT | WTFGQGTKVESK |
| | LVSVSS | LVSVSS | (SEQ ID NO: 61) |
| | EIVLTQSPATLSLSPG | (SEQ ID NO: 1) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSHWPP | | |
| | WTFGQGTKVESK | | |
| | (SEQ ID NO: 183) | | |
| VRDN-03061 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKHPP |
| | ARELGRRYFDLWGRGT | ARELGRRYFDLWGRGT | WTFGQGTKVESK |
| | LVSVSS | LVSVSS | (SEQ ID NO: 62) |
| | EIVLTQSPATLSLSPG | (SEQ ID NO: 1) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKHPP | | |
| | WTFGQGTKVESK | | |
| | (SEQ ID NO: 184) | | |
| VRDN-03062 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWHP |
| | ARELGRRYFDLWGRGT | ARELGRRYFDLWGRGT | WTFGQGTKVESK |
| | LVSVSS | LVSVSS | (SEQ ID NO: 63) |
| | EIVLTQSPATLSLSPG | (SEQ ID NO: 1) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWHP | | |
| | WTFGQGTKVESK | | |
| | (SEQ ID NO: 185) | | |
| VRDN-03063 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPH |
| | ARELGRRYFDLWGRGT | ARELGRRYFDLWGRGT | WTFGQGTKVESK |
| | LVSVSS | LVSVSS | (SEQ ID NO: 64) |
| | EIVLTQSPATLSLSPG | (SEQ ID NO: 1) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPH | | |
| | WTFGQGTKVESK | | |
| | (SEQ ID NO: 186) | | |
| VRDN-03064 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | HTFGQGTKVESK |
| | LVSVSS | LVSVSS | (SEQ ID NO: 65) |
| | EIVLTQSPATLSLSPG | (SEQ ID NO: 1) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | HTFGQGTKVESK | | |
| | (SEQ ID NO: 187) | | |
| VRDN-03065 | QVELVESGGGVVQPGR | QVELVESGGGVVQPGR | EIVLTQSPATLSLSPG |
| | SQRLSCAASGFTFSSY | SQRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYFC | LQMNSLRAEDTAVYFC | EDFAVYYCQQRSKWPP |
| | ARELGRRYFDLWGRGT | ARELGRRYFDLWGRGT | WHFGQGTKVESK |
| | LVSVSS | LVSVSS | (SEQ ID NO: 66) |
| | EIVLTQSPATLSLSPG | (SEQ ID NO: 1) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WHFGQGTKVESK | | |
| | (SEQ ID NO: 188) | | |
| VRDN-03066 | QVQLVESGGGVVQPGR | QVQLVESGGGVVQPGR | EIVLTQSPATLSLSPG |
| | SLRLSCAASGFTFSDY | SLRLSCAASGFTFSDY | ERATLSCRASQSVSSY |
| | AMHWVRQAPGKGLEWV | AMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 67) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 189) | | |
| VRDN-03067 | QVQLVESGGGVVQPGR | QVQLVESGGGVVQPGR | EIVLTQSPATLSLSPG |
| | SLRLSCAASGFTFSDY | SLRLSCAASGFTFSDY | ERATLSCRASQSVSSY |
| | YMSWVRQAPGKGLEWV | YMSWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 68) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 190) | | |
| VRDN-03068 | QVQLVESGGGVVQPGR | QVQLVESGGGVVQPGR | EIVLTQSPATLSLSPG |
| | SLRLSCAASGFTFSDY | SLRLSCAASGFTFSDY | ERATLSCRASQSVSSY |
| | GMSWVRQAPGKGLEWV | GMSWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 69) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 191) | | |
| VRDN-03069 | QVQLVESGGGVVQPGR | QVQLVESGGGVVQPGR | EIVLTQSPATLSLSPG |
| | SLRLSCAASGFTFSDY | SLRLSCAASGFTFSDY | ERATLSCRASQSVSSY |
| | TMHWVRQAPGKGLEWV | TMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 70) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 192) | | |
| VRDN-03070 | QVQLVESGGGVVQPGR | QVQLVESGGGVVQPGR | EIVLTQSPATLSLSPG |
| | SLRLSCAASGFTFSDH | SLRLSCAASGFTFSDH | ERATLSCRASQSVSSY |
| | YMDWVRQAPGKGLEWV | YMDWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 71) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 193) | | |
| VRDN-03071 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | DMHWVRQAPGKGLEWV | DMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 72) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 194) | | |
| VRDN-03072 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | SMHWVRQAPGKGLEWV | SMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 73) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 195) | | |
| VRDN-03073 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | SMNWVRQAPGKGLEWV | SMNWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 74) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 196) | | |
| VRDN-03074 | QVQLVESGGGVVQPGR | QVQLVESGGGVVQPGR | EIVLTQSPATLSLSPG |
| | SLRLSCAASGFTFSDY | SLRLSCAASGFTFSDY | ERATLSCRASQSVSSY |
| | AMSWVRQAPGKGLEWV | AMSWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 75) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 197) | | |
| VRDN-03075 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSHYYADSV | AIIWFDGSSHYYADSV | YDASKRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 76) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 198) | | |
| VRDN-03076 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSKYYADSV | AIIWFDGSSKYYADSV | YDASKRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 77) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 199) | | |
| VRDN-03077 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSHYYVDSV | AIIWFDGSSHYYVDSV | YDASKRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 78) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 200) | | |
| VRDN-03078 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSKYYVDSV | AIIWFDGSSKYYVDSV | YDASKRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 79) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 201) | | |
| VRDN-03079 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSHYYPGSV | AIIWFDGSSHYYPGSV | YDASKRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 80) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 202) | | |
| VRDN-03080 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSKYYPGSV | AIIWFDGSSKYYPGSV | YDASKRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 81) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 203) | | |
| VRDN-03081 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AVIWYDGSSHYYADSV | AVIWYDGSSHYYADSV | YDASKRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 82) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 204) | | |
| VRDN-03082 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AVIWYDGSSKYYADSV | AVIWYDGSSKYYADSV | YDASKRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 83) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 205) | | |
| VRDN-03083 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AVIWYDGSSHYYVDSV | AVIWYDGSSHYYVDSV | YDASKRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ IN NO: 84) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 206) | | |
| VRDN-03084 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AVIWYDGSSKYYVDSV | AVIWYDGSSKYYVDSV | YDASKRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 85) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 207) | | |
| VRDN-03085 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AVIWYDGSSHYYPGSV | AVIWYDGSSHYYPGSV | YDASKRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 86) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 208) | | |
| VRDN-03086 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AVIWYDGSSKYYPGSV | AVIWYDGSSKYYPGSV | YDASKRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 8) |
| | EIVLTQSPATLSLSPG | (SEQ ID NO: 87) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 209) | | |
| VRDN-03087 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDIWGRGT | ARELGRRYFDIWGRGT | WTFGQGTKVEIK |
| | LVTVSS | LVTVSS | (SEQ ID NO: 8) |
| | EIVLTQSPATLSLSPG | (SEQ ID NO: 88) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 210) | | |
| VRDN-03088 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSN |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 89) |
| | EIVLTQSPATLSLSPG | (SEQ ID NO: 7) | |
| | ERATLSCRASQSVSSN | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 211) | | |
| VRDN-03089 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSS |
| | GMHWVRQAPGKGLEWV | GMHWVRQAPGKGLEWV | YLAWYQQKPGQAPRLL |
| | AIIWFDGSSTYYADSV | AIIWFDGSSTYYADSV | IYDASKRATGIPARES |
| | RGRFTISRDNSKNTLY | RGRFTISRDNSKNTLY | GSGSGTDFTLTISSLE |
| | LQMNSLRAEDTAVYYC | LQMNSLRAEDTAVYYC | PEDFAVYYCQQRSKWP |
| | ARELGRRYFDLWGRGT | ARELGRRYFDLWGRGT | PWTFGQGTKVEIK |
| | LVTVSS | LVTVSS | (SEQ ID NO: 90) |
| | EIVLTQSPATLSLSPG | (SEQ ID NO: 7) | |
| | ERATLSCRASQSVSSS | | |
| | YLAWYQQKPGQAPRLL | | |
| | IYDASKRATGIPARFS | | |
| | GSGSGTDFTLTISSLE | | |
| | PEDFAVYYCQQRSKWP | | |
| | PWTFGQGTKVEIK | | |
| | (SEQ ID NO: 212) | | |
| VRDN-03090 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQGVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 91) |
| | EIVLTQSPATLSLSPG | (SEQ ID NO: 7) | |
| | ERATLSCRASQGVSSY | | |
| | LAWYQQKPGQAPRLLI | | |
| | YDASKRATGIPARFSG | | |
| | SGSGTDFTLTISSLEP | | |
| | EDFAVYYCQQRSKWPP | | |
| | WTFGQGTKVEIK | | |
| | (SEQ ID NO: 213) | | |
| VRDN-03091 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCGASQSVSSS |
| | GMHWVRQAPGKGLEWV | GMHWVRQAPGKGLEWV | YLAWYQQKPGQAPRLL |
| | AIIWFDGSSTYYADSV | AIIWFDGSSTYYADSV | IYDASKRATGIPARES |
| | RGRFTISRDNSKNTLY | RGRFTISRDNSKNTLY | GSGSGTDFTLTISSLE |
| | LQMNSLRAEDTAVYYC | LQMNSLRAEDTAVYYC | PEDFAVYYCQQRSKWP |
| | ARELGRRYFDLWGRGT | ARELGRRYFDLWGRGT | PWTFGQGTKVEIK |
| | LVTVSS | LVTVSS | (SEQ ID NO: 92) |
| | EIVLTQSPATLSLSPG | (SEQ ID NO: 7) | |
| | ERATLSCGASQSVSSS | | |
| | YLAWYQQKPGQAPRLL | | |
| | IYDASKRATGIPARES | | |
| | GSGSGTDFTLTISSLE | | |
| | PEDFAVYYCQQRSKWP | | |
| | PWTFGQGTKVEIK | | |
| | (SEQ ID NO: 214) | | |
| VRDN-03092 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSS |
| | GMHWVRQAPGKGLEWV | GMHWVRQAPGKGLEWV | YLSWYQQKPGQAPRLL |
| | AIIWFDGSSTYYADSV | AIIWFDGSSTYYADSV | IYDASKRATGIPARES |
| | RGRFTISRDNSKNTLY | RGRFTISRDNSKNTLY | GSGSGTDFTLTISSLE |
| | LQMNSLRAEDTAVYYC | LQMNSLRAEDTAVYYC | PEDFAVYYCQQRSKWP |
| | ARELGRRYFDLWGRGT | ARELGRRYFDLWGRGT | PWTFGQGTKVEIK |
| | LVTVSS | LVTVSS | (SEQ ID NO: 93) |
| | EIVLTQSPATLSLSPG | (SEQ ID NO: 7) | |
| | ERATLSCRASQSVSSS | | |
| | YLSWYQQKPGQAPRLL | | |
| | IYDASKRATGIPARES | | |
| | GSGSGTDFTLTISSLE | | |
| | PEDFAVYYCQQRSKWP | | |
| | PWTFGQGTKVEIK | | |
| | (SEQ ID NO: 215) | | |
| VRDN-03093 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASNRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 94) |
| | EIVLTQSPATLSLSPG | (SEQ ID NO: 7) | |
| | ERATLSCRASQSVSSY | | |
| | LAWYQQKPGQAPRLLI | | |
| | YDASNRATGIPARFSG | | |
| | SGSGTDFTLTISSLEP | | |
| | EDFAVYYCQQRSKWPP | | |
| | WTFGQGTKVEIK | | |
| | (SEQ ID NO: 216) | | |
| VRDN-03094 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YGASTRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 95) |
| | EIVLTQSPATLSLSPG | (SEQ ID NO: 7) | |
| | ERATLSCRASQSVSSY | | |
| | LAWYQQKPGQAPRLLI | | |
| | YGASTRATGIPARFSG | | |
| | SGSGTDFTLTISSLEP | | |
| | EDFAVYYCQQRSKWPP | | |
| | WTFGQGTKVEIK | | |
| | (SEQ ID NO: 217) | | |
| VRDN-03095 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YGASSRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 96) |
| | EIVLTQSPATLSLSPG | (SEQ ID NO: 7) | |
| | ERATLSCRASQSVSSY | | |
| | LAWYQQKPGQAPRLLI | | |
| | YGASSRATGIPARFSG | | |
| | SGSGTDFTLTISSLEP | | |
| | EDFAVYYCQQRSKWPP | | |
| | WTFGQGTKVEIK | | |
| | (SEQ ID NO: 218) | | |
| VRDN-03096 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASSRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSKWPP |
| | ARELGRRYFDLWGRGT | ARELGRRYFDLWGRGT | WTFGQGTKVEIK |
| | LVTVSS | LVTVSS | (SEQ ID NO: 97) |
| | EIVLTQSPATLSLSPG | (SEQ ID NO: 7) | |
| | ERATLSCRASQSVSSY | | |
| | LAWYQQKPGQAPRLLI | | |
| | YDASSRATGIPARFSG | | |
| | SGSGTDFTLTISSLEP | | |
| | EDFAVYYCQQRSKWPP | | |
| | WTFGQGTKVEIK | | |
| | (SEQ ID NO: 219) | | |
| VRDN-03097 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQSVSSY |
| | GMHWVRQAPGKGLEWV | GMHWVRQAPGKGLEWV | LAWYQQKPGQAPRLLI |
| | AIIWFDGSSTYYADSV | AIIWFDGSSTYYADSV | YDASKRATGIPARFSG |
| | RGRFTISRDNSKNTLY | RGRFTISRDNSKNTLY | SGSGTDFTLTISSLEP |
| | LQMNSLRAEDTAVYYC | LQMNSLRAEDTAVYYC | EDFAVYYCQQRSRWPP |
| | ARELGRRYFDLWGRGT | ARELGRRYFDLWGRGT | WTFGQGTKVEIK |
| | LVTVSS | LVTVSS | (SEQ ID NO: 98) |
| | EIVLTQSPATLSLSPG | (SEQ ID NO: 7) | |
| | ERATLSCRASQSVSSY | | |
| | LAWYQQKPGQAPRLLI | | |
| | YDASKRATGIPARFSG | | |
| | SGSGTDFTLTISSLEP | | |
| | EDFAVYYCQQRSRWPP | | |
| | WTFGQGTKVEIK | | |
| | (SEQ ID NO: 220) | | |
| VRDN-03098 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQGVSSY |
| | SMHWVRQAPGKGLEWV | SMHWVRQAPGKGLEWV | LAWYQQKPGQAPRLLI |
| | AIIWFDGSSKYYADSV | AIIWFDGSSKYYADSV | YGASTRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 100) |
| | EIVLTQSPATLSLSPG | (SEQ ID NO: 99) | |
| | ERATLSCRASQGVSSY | | |
| | LAWYQQKPGQAPRLLI | | |
| | YGASTRATGIPARFSG | | |
| | SGSGTDFTLTISSLEP | | |
| | EDFAVYYCQQRSRWPP | | |
| | WTFGQGTKLEIK | | |
| | (SEQ ID NO: 221) | | |
| VRDN-03099 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQGVSSY |
| | SMHWVRQAPGKGLEWV | SMHWVRQAPGKGLEWV | LAWYQQKPGQAPRLLI |
| | AIIWFDGSSKYYVDSV | AIIWFDGSSKYYVDSV | YGASTRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 100) |
| | EIVLTQSPATLSLSPG | (SEQ ID NO: 101) | |
| | ERATLSCRASQGVSSY | | |
| | LAWYQQKPGQAPRLLI | | |
| | YGASTRATGIPARFSG | | |
| | SGSGTDFTLTISSLEP | | |
| | EDFAVYYCQQRSRWPP | | |
| | WTFGQGTKLEIK | | |
| | (SEQ ID NO: 222) | | |
| VRDN-03100 | QVQLVESGGGVVQPGR | QVQLVESGGGVVQPGR | EIVLTQSPATLSLSPG |
| | SLRLSCAASGFTFSSY | SLRLSCAASGFTFSSY | ERATLSCRASQGVSSY |
| | SMHWVRQAPGKGLEWV | SMHWVRQAPGKGLEWV | LAWYQQKPGQAPRLLI |
| | AIIWFDGSSKYYPGSV | AIIWFDGSSKYYPGSV | YGASTRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 100) |
| | EIVLTQSPATLSLSPG | (SEQ ID NO: 102) | |
| | ERATLSCRASQGVSSY | | |
| | LAWYQQKPGQAPRLLI | | |
| | YGASTRATGIPARFSG | | |
| | SGSGTDFTLTISSLEP | | |
| | EDFAVYYCQQRSRWPP | | |
| | WTFGQGTKLEIK | | |
| | (SEQ ID NO: 223) | | |
| VRDN-03101 | QVQLVESGGGVVQPGR | QVQLVESGGGVVQPGR | EIVLTQSPATLSLSPG |
| | SLRLSCAASGFTFSDY | SLRLSCAASGFTFSDY | ERATLSCRASQGVSSY |
| | TMHWVRQAPGKGLEWV | TMHWVRQAPGKGLEWV | LAWYQQKPGQAPRLLI |
| | AIIWFDGSSKYYADSV | AIIWFDGSSKYYADSV | YGASTRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 100) |
| | EIVLTQSPATLSLSPG | (SEQ ID NO: 103) | |
| | ERATLSCRASQGVSSY | | |
| | LAWYQQKPGQAPRLLI | | |
| | YGASTRATGIPARFSG | | |
| | SGSGTDFTLTISSLEP | | |
| | EDFAVYYCQQRSRWPP | | |
| | WTFGQGTKLEIK | | |
| | (SEQ ID NO: 224) | | |
| VRDN-03102 | QVQLVESGGGVVQPGR | QVQLVESGGGVVQPGR | EIVLTQSPATLSLSPG |
| | SLRLSCAASGFTFSDY | SLRLSCAASGFTFSDY | ERATLSCRASQGVSSY |
| | TMHWVRQAPGKGLEWV | TMHWVRQAPGKGLEWV | LAWYQQKPGQAPRLLI |
| | AIIWFDGSSKYYVDSV | AIIWFDGSSKYYVDSV | YGASTRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 100) |
| | EIVLTQSPATLSLSPG | (SEQ ID NO: 104) | |
| | ERATLSCRASQGVSSY | | |
| | LAWYQQKPGQAPRLLI | | |
| | YGASTRATGIPARFSG | | |
| | SGSGTDFTLTISSLEP | | |
| | EDFAVYYCQQRSRWPP | | |
| | WTFGQGTKLEIK | | |
| | (SEQ ID NO: 225) | | |
| VRDN-03103 | QVQLVESGGGVVQPGR | QVQLVESGGGVVQPGR | EIVLTQSPATLSLSPG |
| | SLRLSCAASGFTFSDY | SLRLSCAASGFTFSDY | ERATLSCRASQGVSSY |
| | TMHWVRQAPGKGLEWV | TMHWVRQAPGKGLEWV | LAWYQQKPGQAPRLLI |
| | AIIWFDGSSKYYPGSV | AIIWFDGSSKYYPGSV | YGASTRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 100) |
| | EIVLTQSPATLSLSPG | (SEQ ID NO: 105) | |
| | ERATLSCRASQGVSSY | | |
| | LAWYQQKPGQAPRLLI | | |
| | YGASTRATGIPARFSG | | |
| | SGSGTDFTLTISSLEP | | |
| | EDFAVYYCQQRSRWPP | | |
| | WTFGQGTKLEIK | | |
| | (SEQ ID NO: 226) | | |
| VRDN-03104 | QVQLVESGGGVVQPGR | QVQLVESGGGVVQPGR | EIVLTQSPATLSLSPG |
| | SLRLSCAASGFTFSDH | SLRLSCAASGFTFSDH | ERATLSCRASQGVSSY |
| | YMDWVRQAPGKGLEWV | YMDWVRQAPGKGLEWV | LAWYQQKPGQAPRLLI |
| | AIIWFDGSSKYYADSV | AIIWFDGSSKYYADSV | YGASTRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 100) |
| | EIVLTQSPATLSLSPG | (SEQ ID NO: 106) | |
| | ERATLSCRASQGVSSY | | |
| | LAWYQQKPGQAPRLLI | | |
| | YGASTRATGIPARFSG | | |
| | SGSGTDFTLTISSLEP | | |
| | EDFAVYYCQQRSRWPP | | |
| | WTFGQGTKLEIK | | |
| | (SEQ ID NO: 227) | | |
| VRDN-03105 | QVQLVESGGGVVQPGR | QVQLVESGGGVVQPGR | EIVLTQSPATLSLSPG |
| | SLRLSCAASGFTFSDH | SLRLSCAASGFTFSDH | ERATLSCRASQGVSSY |
| | YMDWVRQAPGKGLEWV | YMDWVRQAPGKGLEWV | LAWYQQKPGQAPRLLI |
| | AIIWFDGSSKYYVDSV | AIIWFDGSSKYYVDSV | YGASTRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 100) |
| | EIVLTQSPATLSLSPG | (SEQ ID NO: 107) | |
| | ERATLSCRASQGVSSY | | |
| | LAWYQQKPGQAPRLLI | | |
| | YGASTRATGIPARFSG | | |
| | SGSGTDFTLTISSLEP | | |
| | EDFAVYYCQQRSRWPP | | |
| | WTFGQGTKLEIK | | |
| | (SEQ ID NO: 228) | | |
| VRDN-03106 | QVQLVESGGGVVQPGR | QVQLVESGGGVVQPGR | EIVLTQSPATLSLSPG |
| | SLRLSCAASGFTFSDH | SLRLSCAASGFTFSDH | ERATLSCRASQGVSSY |
| | YMDWVRQAPGKGLEWV | YMDWVRQAPGKGLEWV | LAWYQQKPGQAPRLLI |
| | AIIWFDGSSKYYPGSV | AIIWFDGSSKYYPGSV | YGASTRATGIPARFSG |
| | KGRFTISRDNSKNTLY | KGRFTISRDNSKNTLY | SGSGTDFTLTISSLEP |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 100) |
| | EIVLTQSPATLSLSPG | (SEQ ID NO: 108) | |
| | ERATLSCRASQGVSSY | | |
| | LAWYQQKPGQAPRLLI | | |
| | YGASTRATGIPARFSG | | |
| | SGSGTDFTLTISSLEP | | |
| | EDFAVYYCQQRSRWPP | | |
| | WTFGQGTKLEIK | | |
| | (SEQ ID NO: 229) | | |
| VRDN-03107 | QVQLVESGGGVVQPGR | QVQLVESGGGVVQPGR | EIVMTQSPATLSVSPG |
| | SLRLSCAASGFTFSSY | SLRLSCAASGFTFSSY | ERATLSCRASQGVSSY |
| | SMHWVRQAPGKGLEWV | SMHWVRQAPGKGLEWV | LAWYQQKPGQAPRLLI |
| | AIIWFDGSSKYYADSV | AIIWFDGSSKYYADSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 109) |
| | EIVMTQSPATLSVSPG | (SEQ ID NO: 99) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 230) | | |
| VRDN-03108 | QVQLVESGGGVVQPGR | QVQLVESGGGVVQPGR | EIVMTQSPATLSVSPG |
| | SLRLSCAASGFTFSSY | SLRLSCAASGFTFSSY | ERATLSCRASQGVSSY |
| | SMHWVRQAPGKGLEWV | SMHWVRQAPGKGLEWV | LAWYQQKPGQAPRLLI |
| | AIIWFDGSSKYYVDSV | AIIWFDGSSKYYVDSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 109) |
| | EIVMTQSPATLSVSPG | (SEQ ID NO: 101) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 231) | | |
| VRDN-03109 | QVQLVESGGGVVQPGR | QVQLVESGGGVVQPGR | EIVMTQSPATLSVSPG |
| | SLRLSCAASGFTFSSY | SLRLSCAASGFTFSSY | ERATLSCRASQGVSSY |
| | SMHWVRQAPGKGLEWV | SMHWVRQAPGKGLEWV | LAWYQQKPGQAPRLLI |
| | AIIWFDGSSKYYPGSV | AIIWFDGSSKYYPGSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 109) |
| | EIVMTQSPATLSVSPG | (SEQ ID NO: 102) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 232) | | |
| VRDN-03110 | QVQLVESGGGVVQPGR | QVQLVESGGGVVQPGR | EIVMTQSPATLSVSPG |
| | SLRLSCAASGFTFSDY | SLRLSCAASGFTFSDY | ERATLSCRASQGVSSY |
| | TMHWVRQAPGKGLEWV | TMHWVRQAPGKGLEWV | LAWYQQKPGQAPRLLI |
| | AIIWFDGSSKYYADSV | AIIWFDGSSKYYADSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 109) |
| | EIVMTQSPATLSVSPG | (SEQ ID NO: 103) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 233) | | |
| VRDN-03111 | QVQLVESGGGVVQPGR | QVQLVESGGGVVQPGR | EIVMTQSPATLSVSPG |
| | SLRLSCAASGFTFSDY | SLRLSCAASGFTFSDY | ERATLSCRASQGVSSY |
| | TMHWVRQAPGKGLEWV | TMHWVRQAPGKGLEWV | LAWYQQKPGQAPRLLI |
| | AIIWFDGSSKYYVDSV | AIIWFDGSSKYYVDSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 109) |
| | EIVMTQSPATLSVSPG | (SEQ ID NO: 104) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 234) | | |
| VRDN-03112 | QVQLVESGGGVVQPGR | QVQLVESGGGVVQPGR | EIVMTQSPATLSVSPG |
| | SLRLSCAASGFTFSDY | SLRLSCAASGFTFSDY | ERATLSCRASQGVSSY |
| | TMHWVRQAPGKGLEWV | TMHWVRQAPGKGLEWV | LAWYQQKPGQAPRLLI |
| | AIIWFDGSSKYYPGSV | AIIWFDGSSKYYPGSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 109) |
| | EIVMTQSPATLSVSPG | (SEQ ID NO: 105) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 235) | | |
| VRDN-03113 | QVQLVESGGGVVQPGR | QVQLVESGGGVVQPGR | EIVMTQSPATLSVSPG |
| | SLRLSCAASGFTFSDH | SLRLSCAASGFTFSDH | ERATLSCRASQGVSSY |
| | YMDWVRQAPGKGLEWV | YMDWVRQAPGKGLEWV | LAWYQQKPGQAPRLLI |
| | AIIWFDGSSKYYADSV | AIIWFDGSSKYYADSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 109) |
| | EIVMTQSPATLSVSPG | (SEQ ID NO: 106) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 236) | | |
| VRDN-03114 | QVQLVESGGGVVQPGR | QVQLVESGGGVVQPGR | EIVMTQSPATLSVSPG |
| | SLRLSCAASGFTFSDH | SLRLSCAASGFTFSDH | ERATLSCRASQGVSSY |
| | YMDWVRQAPGKGLEWV | YMDWVRQAPGKGLEWV | LAWYQQKPGQAPRLLI |
| | AIIWFDGSSKYYVDSV | AIIWFDGSSKYYVDSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 109) |
| | EIVMTQSPATLSVSPG | (SEQ ID NO: 107) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 237) | | |
| VRDN-03115 | QVQLVESGGGVVQPGR | QVQLVESGGGVVQPGR | EIVMTQSPATLSVSPG |
| | SLRLSCAASGFTFSDH | SLRLSCAASGFTFSDH | ERATLSCRASQGVSSY |
| | YMDWVRQAPGKGLEWV | YMDWVRQAPGKGLEWV | LAWYQQKPGQAPRLLI |
| | AIIWFDGSSKYYPGSV | AIIWFDGSSKYYPGSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 109) |
| | EIVMTQSPATLSVSPG | (SEQ ID NO: 108) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 238) | | |
| VRDN-03116 | EVQLVESGGGVVQPGR | EVQLVESGGGVVQPGR | EIVMTQSPATLSVSPG |
| | SLRLSCAASGFTFSDY | SLRLSCAASGFTFSDY | ERATLSCRASQGVSSY |
| | TMHWVRQAPGKGLEWV | TMHWVRQAPGKGLEWV | LAWYQQKPGQAPRLLI |
| | AIIWFDGSSKYYPGSV | AIIWFDGSSKYYPGSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 109) |
| | EIVMTQSPATLSVSPG | (SEQ ID NO: 110) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 239) | | |
| VRDN-03117 | EVQLVESGGGVVQPGR | EVQLVESGGGVVQPGR | EIVMTQSPATLSVSPG |
| | SLRLSCAASGFTFSSY | SLRLSCAASGFTFSSY | ERATLSCRASQGVSSY |
| | SMHWVRQAPGKGLEWV | SMHWVRQAPGKGLEWV | LAWYQQKPGQAPRLLI |
| | AIIWFDGSNKYYADSV | AIIWFDGSNKYYADSV | YGASTRATGIPARFSG |
| | KSRFTISRDNSKNTLY | KSRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGQGTKLEIK |
| | LVTVSS | LVTVSS | (SEQ ID NO: 109) |
| | EIVMTQSPATLSVSPG | (SEQ ID NO: 111) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 240) | | |
| VRDN-03118 | EVQLVESGGGVVQPGR | EVQLVESGGGVVQPGR | EIVMTQSPATLSVSPG |
| | SLRLSCAASGFTFSSY | SLRLSCAASGFTFSSY | ERATLSCRASQGVSSY |
| | SMHWVRQAPGKGLEWV | SMHWVRQAPGKGLEWV | LAWYQQKPGQAPRLLI |
| | AIIWFDGSSKYYVDSV | AIIWFDGSSKYYVDSV | YGASTRATGIPARFSG |
| | KSRFTISRDNSKNTLY | KSRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGQGTKLEIK |
| | LVTVSS | LVTVSS | (SEQ ID NO: 109) |
| | EIVMTQSPATLSVSPG | (SEQ ID NO: 112) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 241) | | |
| VRDN-03119 | EVQLVESGGGVVQPGR | EVQLVESGGGVVQPGR | EIVMTQSPATLSVSPG |
| | SLRLSCAASGFTFSSY | SLRLSCAASGFTFSSY | ERATLSCRASQGVSSY |
| | SMHWVRQAPGKGLEWV | SMHWVRQAPGKGLEWV | LAWYQQKPGQAPRLLI |
| | AIIWFDGSEKYYVDSV | AIIWFDGSEKYYVDSV | YGASTRATGIPARFSG |
| | KSRFTISRDNSKNTLY | KSRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGQGTKLEIK |
| | LVTVSS | LVTVSS | (SEQ ID NO: 109) |
| | EIVMTQSPATLSVSPG | (SEQ ID NO: 113) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 242) | | |
| VRDN-03120 | EVQLVESGGGVVQPGR | EVQLVESGGGVVQPGR | EIVMTQSPATLSVSPG |
| | SLRLSCAASGFTFSSY | SLRLSCAASGFTFSSY | ERATLSCRASQGVSSY |
| | SMHWVRQAPGKGLEWV | SMHWVRQAPGKGLEWV | LAWYQQKPGQAPRLLI |
| | AVIWYDGSSKYYADSV | AVIWYDGSSKYYADSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGQGTKLEIK |
| | LVTVSS | LVTVSS | (SEQ ID NO: 109) |
| | EIVMTQSPATLSVSPG | (SEQ ID NO: 114) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 243) | | |
| VRDN-03121 | EVQLVESGGGVVQPGR | EVQLVESGGGVVQPGR | EIVMTQSPATLSVSPG |
| | SLRLSCAASGFTFSSY | SLRLSCAASGFTFSSY | ERATLSCRASQGVSSY |
| | SMHWVRQAPGKGLEWV | SMHWVRQAPGKGLEWV | LAWYQQKPGQAPRLLI |
| | AVIWYDGSSKYYADSV | AVIWYDGSSKYYADSV | YGASTRATGIPARFSG |
| | KSRFTISRDNSKNTLY | KSRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGQGTKLEIK |
| | LVTVSS | LVTVSS | (SEQ ID NO: 109) |
| | EIVMTQSPATLSVSPG | (SEQ ID NO: 115) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 244) | | |
| VRDN-03122 | EVQLVESGGGVVQPGR | EVQLVESGGGVVQPGR | EIVMTQSPATLSVSPG |
| | SLRLSCAASGFTFSSY | SLRLSCAASGFTFSSY | ERATLSCRASQGVSSY |
| | SMHWVRQAPGKGLEWV | SMHWVRQAPGKGLEWV | LAWYQQKPGQAPRLLI |
| | AVIWYDGSSKYYADSV | AVIWYDGSSKYYADSV | YGASTRATGIPARFSG |
| | KSRFTISRDNSKNTLY | KSRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGQGTKLEIK |
| | LVTVSS | LVTVSS | (SEQ ID NO: 109) |
| | EIVMTQSPATLSVSPG | (SEQ ID NO: 116) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 245) | | |
| VRDN-03123 | EVQLVESGGGVVQPGR | EVQLVESGGGVVQPGR | EIVMTQSPATLSVSPG |
| | SLRLSCAASGFTFDDY | SLRLSCAASGFTFDDY | ERATLSCRASQGVSSY |
| | AMHWVRQAPGKGLEWV | AMHWVRQAPGKGLEWV | LAWYQQKPGQAPRLLI |
| | AIIWFDGSSKYYADSV | AIIWFDGSSKYYADSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGQGTKLEIK |
| | LVTVSS | LVTVSS | (SEQ ID NO: 109) |
| | EIVMTQSPATLSVSPG | (SEQ ID NO: 117) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 246) | | |
| VRDN-03124 | EVQLVESGGGVVQPGR | EVQLVESGGGVVQPGR | EIVMTQSPATLSVSPG |
| | SLRLSCAASGFTFDDY | SLRLSCAASGFTFDDY | ERATLSCRASQGVSSY |
| | TMHWVRQAPGKGLEWV | TMHWVRQAPGKGLEWV | LAWYQQKPGQAPRLLI |
| | AIIWFDGSSKYYADSV | AIIWFDGSSKYYADSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGQGTKLEIK |
| | LVTVSS | LVTVSS | (SEQ ID NO: 109) |
| | EIVMTQSPATLSVSPG | (SEQ ID NO: 118) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 247) | | |
| VRDN-03125 | EVQLVESGGGVVQPGR | EVQLVESGGGVVQPGR | EIVMTQSPATLSVSPG |
| | SLRLSCAASGFTFDDY | SLRLSCAASGFTFDDY | ERATLSCRASQGVSSY |
| | AMHWVRQAPGKGLEWV | AMHWVRQAPGKGLEWV | LAWYQQKPGQAPRLLI |
| | AVIWYDGSSKYYADSV | AVIWYDGSSKYYADSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGQGTKLEIK |
| | LVTVSS | LVTVSS | (SEQ ID NO: 109) |
| | EIVMTQSPATLSVSPG | (SEQ ID NO: 119) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 248) | | |
| VRDN-03126 | EVQLVESGGGVVQPGR | EVQLVESGGGVVQPGR | EIVMTQSPATLSVSPG |
| | SLRLSCAASGFTFDDY | SLRLSCAASGFTFDDY | ERATLSCRASQGVSSY |
| | TMHWVRQAPGKGLEWV | TMHWVRQAPGKGLEWV | LAWYQQKPGQAPRLLI |
| | AVIWYDGSSKYYADSV | AVIWYDGSSKYYADSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGQGTKLEIK |
| | LVTVSS | LVTVSS | (SEQ ID NO: 109) |
| | EIVMTQSPATLSVSPG | (SEQ ID NO: 120) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 249) | | |
| VRDN-03127 | EVQLVESGGGVVQPGR | EVQLVESGGGVVQPGR | EIVMTQSPATLSVSPG |
| | SLRLSCAASGFTFSDY | SLRLSCAASGFTFSDY | ERATLSCRASQGVSSY |
| | TMHWVRQAPGKGLEWV | TMHWVRQAPGKGLEWV | LAWYQQKPGQAPRLLI |
| | AIIWFDGSSKYYADSV | AIIWFDGSSKYYADSV | YGASTRATGIPARFSG |
| | KSRFTISRDNSKNTLY | KSRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGQGTKLEIK |
| | LVTVSS | LVTVSS | (SEQ ID NO: 109) |
| | EIVMTQSPATLSVSPG | (SEQ ID NO: 121) | |
| | ERATLSCRASQGVSSY | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGQGTKLEIK | | |
| | (SEQ ID NO: 250) | | |
| VRDN-03128 | EVQLVESGGGVVQPGR | EVQLVESGGGVVQPGR | EIVMTQSPATLSVSPG |
| | SLRLSCAASGFTFSDY | SLRLSCAASGFTFSDY | ERATLSCRASQGVSSN |
| | TMHWVRQAPGKGLEWV | TMHWVRQAPGKGLEWV | LAWYQQKPGQAPRLLI |
| | AIIWFDGSSKYYPGSV | AIIWFDGSSKYYPGSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGGGTKLEIK |
| | LVTVSS | LVTVSS | (SEQ ID NO: 122) |
| | EIVMTQSPATLSVSPG | (SEQ ID NO: 110) | |
| | ERATLSCRASQGVSSN | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGGGTKLEIK | | |
| | (SEQ ID NO: 251) | | |
| VRDN-03129 | EVQLVESGGGVVQPGR | EVQLVESGGGVVQPGR | EIVMTQSPATLSVSPG |
| | SLRLSCAASGFTFSSY | SLRLSCAASGFTFSSY | ERATLSCRASQGVSSN |
| | SMHWVRQAPGKGLEWV | SMHWVRQAPGKGLEWV | LAWYQQKPGQAPRLLI |
| | AIIWFDGSNKYYADSV | AIIWFDGSNKYYADSV | YGASTRATGIPARFSG |
| | KSRFTISRDNSKNTLY | KSRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGGGTKLEIK |
| | LVTVSS | LVTVSS | (SEQ ID NO: 122) |
| | EIVMTQSPATLSVSPG | (SEQ ID NO: 111) | |
| | ERATLSCRASQGVSSN | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGGGTKLEIK | | |
| | (SEQ ID NO: 252) | | |
| VRDN-03130 | EVQLVESGGGVVQPGR | EVQLVESGGGVVQPGR | EIVMTQSPATLSVSPG |
| | SLRLSCAASGFTFSSY | SLRLSCAASGFTFSSY | ERATLSCRASQGVSSN |
| | SMHWVRQAPGKGLEWV | SMHWVRQAPGKGLEWV | LAWYQQKPGQAPRLLI |
| | AIIWFDGSSKYYVDSV | AIIWFDGSSKYYVDSV | YGASTRATGIPARFSG |
| | KSRFTISRDNSKNTLY | KSRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGGGTKLEIK |
| | LVTVSS | LVTVSS | (SEQ ID NO: 122) |
| | EIVMTQSPATLSVSPG | (SEQ ID NO: 112) | |
| | ERATLSCRASQGVSSN | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGGGTKLEIK | | |
| | (SEQ ID NO: 253) | | |
| VRDN-03131 | EVQLVESGGGVVQPGR | EVQLVESGGGVVQPGR | EIVMTQSPATLSVSPG |
| | SLRLSCAASGFTFSSY | SLRLSCAASGFTFSSY | ERATLSCRASQGVSSN |
| | SMHWVRQAPGKGLEWV | SMHWVRQAPGKGLEWV | LAWYQQKPGQAPRLLI |
| | AIIWFDGSEKYYVDSV | AIIWFDGSEKYYVDSV | YGASTRATGIPARFSG |
| | KSRFTISRDNSKNTLY | KSRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGGGTKLEIK |
| | LVTVSS | LVTVSS | (SEQ ID NO: 122) |
| | EIVMTQSPATLSVSPG | (SEQ ID NO: 113) | |
| | ERATLSCRASQGVSSN | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGGGTKLEIK | | |
| | (SEQ ID NO: 254) | | |
| VRDN-03132 | EVQLVESGGGVVQPGR | EVQLVESGGGVVQPGR | EIVMTQSPATLSVSPG |
| | SLRLSCAASGFTFSSY | SLRLSCAASGFTFSSY | ERATLSCRASQGVSSN |
| | SMHWVRQAPGKGLEWV | SMHWVRQAPGKGLEWV | LAWYQQKPGQAPRLLI |
| | AVIWYDGSSKYYADSV | AVIWYDGSSKYYADSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGGGTKLEIK |
| | LVTVSS | LVTVSS | (SEQ ID NO: 122) |
| | EIVMTQSPATLSVSPG | (SEQ ID NO: 114) | |
| | ERATLSCRASQGVSSN | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGGGTKLEIK | | |
| | (SEQ ID NO: 255) | | |
| VRDN-03133 | EVQLVESGGGVVQPGR | EVQLVESGGGVVQPGR | EIVMTQSPATLSVSPG |
| | SLRLSCAASGFTFSSY | SLRLSCAASGFTFSSY | ERATLSCRASQGVSSN |
| | SMHWVRQAPGKGLEWV | SMHWVRQAPGKGLEWV | LAWYQQKPGQAPRLLI |
| | AVIWYDGSSKYYADSV | AVIWYDGSSKYYADSV | YGASTRATGIPARFSG |
| | KSRFTISRDNSKNTLY | KSRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGRGT | ARELGRRYFDIWGRGT | WTFGGGTKLEIK |
| | LVTVSS | LVTVSS | (SEQ ID NO: 122) |
| | EIVMTQSPATLSVSPG | (SEQ ID NO: 115) | |
| | ERATLSCRASQGVSSN | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGGGTKLEIK | | |
| | (SEQ ID NO: 256) | | |
| VRDN-03134 | EVQLVESGGGVVQPGR | EVQLVESGGGVVQPGR | EIVMTQSPATLSVSPG |
| | SLRLSCAASGFTFSSY | SLRLSCAASGFTFSSY | ERATLSCRASQGVSSN |
| | SMHWVRQAPGKGLEWV | SMHWVRQAPGKGLEWV | LAWYQQKPGQAPRLLI |
| | AVIWYDGSSKYYADSV | AVIWYDGSSKYYADSV | YGASTRATGIPARFSG |
| | KSRFTISRDNSKNTLY | KSRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGGGTKLEIK |
| | LVTVSS | LVTVSS | (SEQ ID NO: 122) |
| | EIVMTQSPATLSVSPG | (SEQ ID NO: 116) | |
| | ERATLSCRASQGVSSN | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGGGTKLEIK | | |
| | (SEQ ID NO: 257) | | |
| VRDN-03135 | EVQLVESGGGVVQPGR | EVQLVESGGGVVQPGR | EIVMTQSPATLSVSPG |
| | SLRLSCAASGFTFDDY | SLRLSCAASGFTFDDY | ERATLSCRASQGVSSN |
| | AMHWVRQAPGKGLEWV | AMHWVRQAPGKGLEWV | LAWYQQKPGQAPRLLI |
| | AIIWFDGSSKYYADSV | AIIWFDGSSKYYADSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGGGTKLEIK |
| | LVTVSS | LVTVSS | (SEQ ID NO: 122) |
| | EIVMTQSPATLSVSPG | (SEQ ID NO: 117) | |
| | ERATLSCRASQGVSSN | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGGGTKLEIK | | |
| | (SEQ ID NO: 258) | | |
| VRDN-03136 | EVQLVESGGGVVQPGR | EVQLVESGGGVVQPGR | EIVMTQSPATLSVSPG |
| | SLRLSCAASGFTFDDY | SLRLSCAASGFTFDDY | ERATLSCRASQGVSSN |
| | TMHWVRQAPGKGLEWV | TMHWVRQAPGKGLEWV | LAWYQQKPGQAPRLLI |
| | AIIWFDGSSKYYADSV | AIIWFDGSSKYYADSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGGGTKLEIK |
| | LVTVSS | LVTVSS | (SEQ ID NO: 122) |
| | EIVMTQSPATLSVSPG | (SEQ ID NO: 118) | |
| | ERATLSCRASQGVSSN | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGGGTKLEIK | | |
| | (SEQ ID NO: 259) | | |
| VRDN-03137 | EVQLVESGGGVVQPGR | EVQLVESGGGVVQPGR | EIVMTQSPATLSVSPG |
| | SLRLSCAASGFTFDDY | SLRLSCAASGFTFDDY | ERATLSCRASQGVSSN |
| | AMHWVRQAPGKGLEWV | AMHWVRQAPGKGLEWV | LAWYQQKPGQAPRLLI |
| | AVIWYDGSSKYYADSV | AVIWYDGSSKYYADSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGGGTKLEIK |
| | LVTVSS | LVTVSS | (SEQ ID NO: 122) |
| | EIVMTQSPATLSVSPG | (SEQ ID NO: 119) | |
| | ERATLSCRASQGVSSN | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGGGTKLEIK | | |
| | (SEQ ID NO: 260) | | |
| VRDN-03138 | EVQLVESGGGVVQPGR | EVQLVESGGGVVQPGR | EIVMTQSPATLSVSPG |
| | SLRLSCAASGFTFDDY | SLRLSCAASGFTFDDY | ERATLSCRASQGVSSN |
| | TMHWVRQAPGKGLEWV | TMHWVRQAPGKGLEWV | LAWYQQKPGQAPRLLI |
| | AVIWYDGSSKYYADSV | AVIWYDGSSKYYADSV | YGASTRATGIPARFSG |
| | KGRFTISRDNSKNTLY | KGRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGGGTKLEIK |
| | LVTVSS | LVTVSS | (SEQ ID NO: 122) |
| | EIVMTQSPATLSVSPG | (SEQ ID NO: 120) | |
| | ERATLSCRASQGVSSN | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGGGTKLEIK | | |
| | (SEQ ID NO: 261) | | |
| VRDN-03139 | EVQLVESGGGVVQPGR | EVQLVESGGGVVQPGR | EIVMTQSPATLSVSPG |
| | SLRLSCAASGFTFSDY | SLRLSCAASGFTFSDY | ERATLSCRASQGVSSN |
| | TMHWVRQAPGKGLEWV | TMHWVRQAPGKGLEWV | LAWYQQKPGQAPRLLI |
| | AIIWFDGSSKYYADSV | AIIWFDGSSKYYADSV | YGASTRATGIPARFSG |
| | KSRFTISRDNSKNTLY | KSRFTISRDNSKNTLY | SGSGTEFTLTISSLQS |
| | LQMNSLRAEDTAVYYC | LQMNSLRAEDTAVYYC | EDFAVYYCQQRSRWPP |
| | ARELGRRYFDIWGQGT | ARELGRRYFDIWGQGT | WTFGGGTKLEIK |
| | LVTVSS | LVTVSS | (SEQ ID NO: 122) |
| | EIVMTQSPATLSVSPG | (SEQ ID NO: 121) | |
| | ERATLSCRASQGVSSN | | |
| | LAWYQQKPGQAPRLLI | | |
| | YGASTRATGIPARFSG | | |
| | SGSGTEFTLTISSLQS | | |
| | EDFAVYYCQQRSRWPP | | |
| | WTFGGGTKLEIK | | |
| | (SEQ ID NO: 262) | | |

As provided for herein, the heavy chain can be linked to a Fe region, including those with mutations that can affect the half-life of the antibody. Non-limiting mutations in the Fc region are provided for herein.

In some embodiments, the LC and HC may be combined with the VH and VL domains provided herein, with or without constant regions. The constant regions can be replaced as provided for herein. The VH and VL regions can be used to form an antibody as provided for herein. The VH and the VL sequences can be in any format, including, but not limited to a scFv format where the VH and VL regions are linked with a peptide linker. Examples of peptide linkers that can be used to link various peptides provided for herein include, but are not limited to: (GGGGS)ₙ (SEQ ID NO: 375); (GGGGA)ₙ (SEQ ID NO: 376), or any combination thereof, wherein each n is independently 1-5. In some embodiments, the variable regions are not linked with a peptide linker.

In some embodiments, an antibody, or antigen binding fragment thereof is provided, wherein the antibody or antibody fragment comprises a peptide selected from the following table. In some embodiments, CDRs from VH and VL regions are provided in the following table.

| TYPE | SEQUENCE | ID |
|---|---|---|
| VH CDR 1 | | |
| | SYGMH | SEQ ID NO: 263 |
| | HYGMH | SEQ ID NO: 264 |
| | SHGMH | SEQ ID NO: 265 |
| | SYHMH | SEQ ID NO: 266 |
| | SYGHH | SEQ ID NO: 267 |
| | DYAMH | SEQ ID NO: 268 |
| | DYYMS | SEQ ID NO: 269 |
| | DYGMS | SEQ ID NO: 270 |
| | DYTMH | SEQ ID NO: 271 |
| | DHYMD | SEQ ID NO: 272 |
| | SYDMH | SEQ ID NO: 273 |
| | SYSMH | SEQ ID NO: 274 |
| | SYSMN | SEQ ID NO: 275 |
| | DYAMS | SEQ ID NO: 276 |
| VH CDR2 | | |
| | IIWFDGSSTYYADSVRG | SEQ ID NO: 277 |
| | HIWFDGSSTYYADSVRG | SEQ ID NO: 278 |
| | IHWFDGSSTYYADSVRG | SEQ ID NO: 279 |
| | IIHFDGSSTYYADSVRG | SEQ ID NO: 280 |
| | IIWHDGSSTYYADSVRG | SEQ ID NO: 281 |
| | IIWFHGSSTYYADSVRG | SEQ ID NO: 282 |
| | IIWFDHSSTYYADSVRG | SEQ ID NO: 283 |
| | IIWFDGHSTYYADSVRG | SEQ ID NO: 284 |
| | IIWFDGSHTYYADSVRG | SEQ ID NO: 285 |
| | IIWFDGSSHYYADSVRG | SEQ ID NO: 286 |
| | IIWFDGSSTHYADSVRG | SEQ ID NO: 287 |
| | IIWFDGSSTYHADSVRG | SEQ ID NO: 288 |
| | IIWFDGSSTYYHDSVRG | SEQ ID NO: 289 |
| | IIWFDGSSTYYAHSVRG | SEQ ID NO: 290 |
| | IIWFDGSSTYYAHSVRG | SEQ ID NO: 291 |
| | IIWFDGSSTYYADSHRG | SEQ ID NO: 292 |
| | IIWFDGSSTYYADSVHG | SEQ ID NO: 293 |
| | IIWFDGSSTYYADSVRH | SEQ ID NO: 294 |
| | IIWFDGSSHYYADSVKG | SEQ ID NO: 295 |
| | IIWFDGSSKYYADSVKG | SEQ ID NO: 296 |
| | IIWFDGSSHYYVDSVKG | SEQ ID NO: 297 |
| | IIWFDGSSKYYVDSVKG | SEQ ID NO: 298 |
| | IIWFDGSSHYYPGSVKG | SEQ ID NO: 299 |
| | IIWFDGSSKYYPGSVKG | SEQ ID NO: 300 |
| | VIWYDGSSHYYADSVKG | SEQ ID NO: 301 |
| | VIWYDGSSKYYADSVKG | SEQ ID NO: 302 |
| | VIWYDGSSHYYVDSVKG | SEQ ID NO: 303 |
| | VIWYDGSSKYYVDSVKG | SEQ ID NO: 304 |
| | VIWYDGSSHYYPGSVKG | SEQ ID NO: 305 |
| | VIWYDGSSKYYPGSVKG | SEQ ID NO: 306 |
| | IIWFDGSNKYYADSVKS | SEQ ID NO: 307 |
| | IIWFDGSSKYYVDSVKS | SEQ ID NO: 308 |
| | IIWFDGSEKYYVDSVKS | SEQ ID NO: 309 |
| | IIWFDGSSKYYADSVKS | SEQ ID NO: 310 |
| | VIWYDGSSKYYADSVKS | SEQ ID NO: 397 |
| VH CDR3 | | |
| | ELGRRYFDL | SEQ ID NO: 311 |
| | HLGRRYFDL | SEQ ID NO: 312 |
| | EHGRRYFDL | SEQ ID NO: 313 |
| | ELHRRYFDL | SEQ ID NO: 314 |
| | ELGHRYFDL | SEQ ID NO: 315 |
| | ELGRHYFDL | SEQ ID NO: 316 |
| | ELGRRHFDL | SEQ ID NO: 317 |
| | ELGRRYHDL | SEQ ID NO: 318 |
| | ELGRRYFHL | SEQ ID NO: 319 |
| | ELGRRYFDH | SEQ ID NO: 320 |
| | ELGRRYFDI | SEQ ID NO: 321 |
| VL CDR1 | | |
| | RASQSVSSYLA | SEQ ID NO: 322 |
| | HASQSVSSYLA | SEQ ID NO: 323 |
| | RHSQSVSSYLA | SEQ ID NO: 324 |
| | RAHQSVSSYLA | SEQ ID NO: 325 |
| | RASHSVSSYLA | SEQ ID NO: 326 |
| | RASQHVSSYLA | SEQ ID NO: 327 |
| | RASQSHSSYLA | SEQ ID NO: 328 |
| | RASQSVHSYLA | SEQ ID NO: 329 |
| | RASQSVSHYLA | SEQ ID NO: 330 |
| | RASQSVSSHLA | SEQ ID NO: 331 |
| | RASQSVSSYHA | SEQ ID NO: 332 |
| | RASQSVSSYLH | SEQ ID NO: 333 |
| | RASQSVSSNLA | SEQ ID NO: 334 |
| | RASQSVSSSYLA | SEQ ID NO: 335 |
| | RASQGVSSYLA | SEQ ID NO: 336 |
| | GASQSVSSSYLA | SEQ ID NO: 337 |
| | RASQSVSSSYLS | SEQ ID NO: 338 |
| VL CDR2 | | |
| | DASKRAT | SEQ ID NO: 339 |
| | HASKRAT | SEQ ID NO: 340 |
| | DHSKRAT | SEQ ID NO: 341 |
| | DAHKRAT | SEQ ID NO: 342 |
| | DASHRAT | SEQ ID NO: 343 |
| | DASKHAT | SEQ ID NO: 344 |
| | DASKRHT | SEQ ID NO: 345 |
| | DASKRAH | SEQ ID NO: 346 |
| | DASNRAT | SEQ ID NO: 347 |
| | GASTRAT | SEQ ID NO: 348 |
| | GASSRAT | SEQ ID NO: 349 |
| | DASSRAT | SEQ ID NO: 350 |
| VL CDR3 | | |
| | QQRSKWPPWT | SEQ ID NO: 351 |
| | HqRSKWPPWT | SEQ ID NO: 352 |
| | QHRSKWPPWT | SEQ ID NO: 353 |
| | QQHSKWPPWT | SEQ ID NO: 354 |
| | QQRHKWPPWT | SEQ ID NO: 355 |
| | QQRSHWPPWT | SEQ ID NO: 356 |
| | QQRSKHPPWT | SEQ ID NO: 357 |
| | QqRSKWHPWT | SEQ ID NO: 358 |
| | QQRSKWPHWT | SEQ ID NO: 359 |
| | QQRSKWPPHT | SEQ ID NO: 360 |
| | QQRSKWPPWH | SEQ ID NO: 361 |
| | QQRSRWPPWT | SEQ ID NO: 362 |

In some embodiments, an antibody, or antigen binding fragment thereof is provided, wherein the antibody or antibody fragment comprises the heavy chain variable region and corresponding heavy chain variable region CDRs provided in the following table.

| VH | VH CDR1 | VH CDR2 | VH CDR3 |
|---|---|---|---|
| SEQ ID NO: 1 | SEQ ID NO: 263 | SEQ ID NO: 277 | SEQ ID NO: 311 |
| SEQ ID NO: 3 | SEQ ID NO: 263 | SEQ ID NO: 277 | SEQ ID NO: 311 |
| SEQ ID NO: 4 | SEQ ID NO: 263 | SEQ ID NO: 277 | SEQ ID NO: 311 |
| SEQ ID NO: 5 | SEQ ID NO: 263 | SEQ ID NO: 277 | SEQ ID NO: 311 |
| SEQ ID NO: 6 | SEQ ID NO: 263 | SEQ ID NO: 277 | SEQ ID NO: 311 |
| SEQ ID NO: 7 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 7 | SEQ ID NO: 263 | SEQ ID NO: 277 | SEQ ID NO: 311 |
| SEQ ID NO: 9 | SEQ ID NO: 264 | SEQ ID NO: 277 | SEQ ID NO: 311 |
| SEQ ID NO: 10 | SEQ ID NO: 265 | SEQ ID NO: 277 | SEQ ID NO: 311 |
| SEQ ID NO: 11 | SEQ ID NO: 266 | SEQ ID NO: 277 | SEQ ID NO: 311 |
| SEQ ID NO: 12 | SEQ ID NO: 267 | SEQ ID NO: 277 | SEQ ID NO: 311 |
| SEQ ID NO: 13 | SEQ ID NO: 263 | SEQ ID NO: 278 | SEQ ID NO: 311 |
| SEQ ID NO: 14 | SEQ ID NO: 263 | SEQ ID NO: 279 | SEQ ID NO: 311 |
| SEQ ID NO: 15 | SEQ ID NO: 263 | SEQ ID NO: 280 | SEQ ID NO: 311 |
| SEQ ID NO: 16 | SEQ ID NO: 263 | SEQ ID NO: 281 | SEQ ID NO: 311 |
| SEQ ID NO: 17 | SEQ ID NO: 263 | SEQ ID NO: 282 | SEQ ID NO: 311 |
| SEQ ID NO: 18 | SEQ ID NO: 263 | SEQ ID NO: 283 | SEQ ID NO: 311 |
| SEQ ID NO: 19 | SEQ ID NO: 263 | SEQ ID NO: 284 | SEQ ID NO: 311 |
| SEQ ID NO: 20 | SEQ ID NO: 263 | SEQ ID NO: 285 | SEQ ID NO: 311 |
| SEQ ID NO: 21 | SEQ ID NO: 263 | SEQ ID NO: 286 | SEQ ID NO: 311 |
| SEQ ID NO: 22 | SEQ ID NO: 263 | SEQ ID NO: 287 | SEQ ID NO: 311 |
| SEQ ID NO: 23 | SEQ ID NO: 263 | SEQ ID NO: 288 | SEQ ID NO: 311 |
| SEQ ID NO: 24 | SEQ ID NO: 263 | SEQ ID NO: 289 | SEQ ID NO: 311 |
| SEQ ID NO: 25 | SEQ ID NO: 263 | SEQ ID NO: 290 | SEQ ID NO: 311 |
| SEQ ID NO: 26 | SEQ ID NO: 263 | SEQ ID NO: 291 | SEQ ID NO: 311 |
| SEQ ID NO: 27 | SEQ ID NO: 263 | SEQ ID NO: 292 | SEQ ID NO: 311 |
| SEQ ID NO: 28 | SEQ ID NO: 263 | SEQ ID NO: 293 | SEQ ID NO: 311 |
| SEQ ID NO: 29 | SEQ ID NO: 263 | SEQ ID NO: 294 | SEQ ID NO: 311 |
| SEQ ID NO: 30 | SEQ ID NO: 263 | SEQ ID NO: 277 | SEQ ID NO: 312 |
| SEQ ID NO: 31 | SEQ ID NO: 263 | SEQ ID NO: 277 | SEQ ID NO: 313 |
| SEQ ID NO: 32 | SEQ ID NO: 263 | SEQ ID NO: 277 | SEQ ID NO: 314 |
| SEQ ID NO: 33 | SEQ ID NO: 263 | SEQ ID NO: 277 | SEQ ID NO: 315 |
| SEQ ID NO: 34 | SEQ ID NO: 263 | SEQ ID NO: 277 | SEQ ID NO: 316 |
| SEQ ID NO: 35 | SEQ ID NO: 263 | SEQ ID NO: 277 | SEQ ID NO: 317 |
| SEQ ID NO: 36 | SEQ ID NO: 263 | SEQ ID NO: 277 | SEQ ID NO: 318 |
| SEQ ID NO: 37 | SEQ ID NO: 263 | SEQ ID NO: 277 | SEQ ID NO: 319 |
| SEQ ID NO: 38 | SEQ ID NO: 263 | SEQ ID NO: 277 | SEQ ID NO: 320 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 1 | SEQ ID NO: 263 | SEQ ID NO: 277 | SEQ ID NO: 311 |
| SEQ ID NO: 67 | SEQ ID NO: 268 | SEQ ID NO: 277 | SEQ ID NO: 311 |
| SEQ ID NO: 68 | SEQ ID NO: 269 | SEQ ID NO: 277 | SEQ ID NO: 311 |
| SEQ ID NO: 69 | SEQ ID NO: 270 | SEQ ID NO: 277 | SEQ ID NO: 311 |
| SEQ ID NO: 70 | SEQ ID NO: 271 | SEQ ID NO: 277 | SEQ ID NO: 311 |
| SEQ ID NO: 71 | SEQ ID NO: 272 | SEQ ID NO: 277 | SEQ ID NO: 311 |
| SEQ ID NO: 72 | SEQ ID NO: 273 | SEQ ID NO: 277 | SEQ ID NO: 311 |
| SEQ ID NO: 73 | SEQ ID NO: 274 | SEQ ID NO: 277 | SEQ ID NO: 311 |
| SEQ ID NO: 74 | SEQ ID NO: 275 | SEQ ID NO: 277 | SEQ ID NO: 311 |
| SEQ ID NO: 75 | SEQ ID NO: 276 | SEQ ID NO: 277 | SEQ ID NO: 311 |
| SEQ ID NO: 76 | SEQ ID NO: 263 | SEQ ID NO: 295 | SEQ ID NO: 311 |
| SEQ ID NO: 77 | SEQ ID NO: 263 | SEQ ID NO: 296 | SEQ ID NO: 311 |
| SEQ ID NO: 78 | SEQ ID NO: 263 | SEQ ID NO: 297 | SEQ ID NO: 311 |
| SEQ ID NO: 79 | SEQ ID NO: 263 | SEQ ID NO: 298 | SEQ ID NO: 311 |
| SEQ ID NO: 80 | SEQ ID NO: 263 | SEQ ID NO: 299 | SEQ ID NO: 311 |
| SEQ ID NO: 81 | SEQ ID NO: 263 | SEQ ID NO: 300 | SEQ ID NO: 311 |
| SEQ ID NO: 82 | SEQ ID NO: 263 | SEQ ID NO: 301 | SEQ ID NO: 311 |
| SEQ ID NO: 83 | SEQ ID NO: 263 | SEQ ID NO: 302 | SEQ ID NO: 311 |
| SEQ ID NO: 84 | SEQ ID NO: 263 | SEQ ID NO: 303 | SEQ ID NO: 311 |
| SEQ ID NO: 85 | SEQ ID NO: 263 | SEQ ID NO: 304 | SEQ ID NO: 311 |
| SEQ ID NO: 86 | SEQ ID NO: 263 | SEQ ID NO: 305 | SEQ ID NO: 311 |
| SEQ ID NO: 87 | SEQ ID NO: 263 | SEQ ID NO: 306 | SEQ ID NO: 311 |
| SEQ ID NO: 88 | SEQ ID NO: 263 | SEQ ID NO: 277 | SEQ ID NO: 321 |
| SEQ ID NO: 7 | SEQ ID NO: 263 | SEQ ID NO: 277 | SEQ ID NO: 311 |
| SEQ ID NO: 7 | SEQ ID NO: 263 | SEQ ID NO: 277 | SEQ ID NO: 311 |
| SEQ ID NO: 7 | SEQ ID NO: 263 | SEQ ID NO: 277 | SEQ ID NO: 311 |
| SEQ ID NO: 7 | SEQ ID NO: 263 | SEQ ID NO: 277 | SEQ ID NO: 311 |
| SEQ ID NO: 7 | SEQ ID NO: 263 | SEQ ID NO: 277 | SEQ ID NO: 311 |
| SEQ ID NO: 7 | SEQ ID NO: 263 | SEQ ID NO: 277 | SEQ ID NO: 311 |
| SEQ ID NO: 7 | SEQ ID NO: 263 | SEQ ID NO: 277 | SEQ ID NO: 311 |
| SEQ ID NO: 7 | SEQ ID NO: 263 | SEQ ID NO: 277 | SEQ ID NO: 311 |
| SEQ ID NO: 7 | SEQ ID NO: 263 | SEQ ID NO: 277 | SEQ ID NO: 311 |
| SEQ ID NO: 7 | SEQ ID NO: 263 | SEQ ID NO: 277 | SEQ ID NO: 311 |
| SEQ ID NO: 99 | SEQ ID NO: 274 | SEQ ID NO: 296 | SEQ ID NO: 321 |
| SEQ ID NO: 101 | SEQ ID NO: 274 | SEQ ID NO: 298 | SEQ ID NO: 321 |
| SEQ ID NO: 102 | SEQ ID NO: 274 | SEQ ID NO: 300 | SEQ ID NO: 321 |
| SEQ ID NO: 103 | SEQ ID NO: 271 | SEQ ID NO: 296 | SEQ ID NO: 321 |
| SEQ ID NO: 104 | SEQ ID NO: 271 | SEQ ID NO: 298 | SEQ ID NO: 321 |
| SEQ ID NO: 105 | SEQ ID NO: 271 | SEQ ID NO: 300 | SEQ ID NO: 321 |
| SEQ ID NO: 106 | SEQ ID NO: 272 | SEQ ID NO: 296 | SEQ ID NO: 321 |
| SEQ ID NO: 107 | SEQ ID NO: 272 | SEQ ID NO: 298 | SEQ ID NO: 321 |
| SEQ ID NO: 108 | SEQ ID NO: 272 | SEQ ID NO: 300 | SEQ ID NO: 321 |
| SEQ ID NO: 99 | SEQ ID NO: 274 | SEQ ID NO: 296 | SEQ ID NO: 321 |
| SEQ ID NO: 101 | SEQ ID NO: 274 | SEQ ID NO: 298 | SEQ ID NO: 321 |
| SEQ ID NO: 102 | SEQ ID NO: 274 | SEQ ID NO: 300 | SEQ ID NO: 321 |
| SEQ ID NO: 103 | SEQ ID NO: 271 | SEQ ID NO: 296 | SEQ ID NO: 321 |
| SEQ ID NO: 104 | SEQ ID NO: 271 | SEQ ID NO: 298 | SEQ ID NO: 321 |
| SEQ ID NO: 105 | SEQ ID NO: 271 | SEQ ID NO: 300 | SEQ ID NO: 321 |
| SEQ ID NO: 106 | SEQ ID NO: 272 | SEQ ID NO: 296 | SEQ ID NO: 321 |
| SEQ ID NO: 107 | SEQ ID NO: 272 | SEQ ID NO: 298 | SEQ ID NO: 321 |
| SEQ ID NO: 108 | SEQ ID NO: 272 | SEQ ID NO: 300 | SEQ ID NO: 321 |
| SEQ ID NO: 110 | SEQ ID NO: 271 | SEQ ID NO: 300 | SEQ ID NO: 321 |
| SEQ ID NO: 111 | SEQ ID NO: 274 | SEQ ID NO: 307 | SEQ ID NO: 321 |
| SEQ ID NO: 112 | SEQ ID NO: 274 | SEQ ID NO: 308 | SEQ ID NO: 321 |
| SEQ ID NO: 113 | SEQ ID NO: 274 | SEQ ID NO: 309 | SEQ ID NO: 321 |
| SEQ ID NO: 114 | SEQ ID NO: 274 | SEQ ID NO: 302 | SEQ ID NO: 321 |
| SEQ ID NO: 115 | SEQ ID NO: 274 | SEQ ID NO: 397 | SEQ ID NO: 321 |
| SEQ ID NO: 116 | SEQ ID NO: 274 | SEQ ID NO: 397 | SEQ ID NO: 321 |
| SEQ ID NO: 117 | SEQ ID NO: 268 | SEQ ID NO: 296 | SEQ ID NO: 321 |
| SEQ ID NO: 118 | SEQ ID NO: 271 | SEQ ID NO: 296 | SEQ ID NO: 321 |
| SEQ ID NO: 119 | SEQ ID NO: 268 | SEQ ID NO: 302 | SEQ ID NO: 321 |
| SEQ ID NO: 120 | SEQ ID NO: 271 | SEQ ID NO: 302 | SEQ ID NO: 321 |
| SEQ ID NO: 121 | SEQ ID NO: 271 | SEQ ID NO: 310 | SEQ ID NO: 321 |
| SEQ IS NO: 110 | SEQ ID NO: 271 | SEQ ID NO: 300 | SEQ ID NO: 321 |
| SEQ ID NO: 111 | SEQ ID NO: 274 | SEQ ID NO: 307 | SEQ ID NO: 321 |
| SEQ ID NO: 112 | SEQ ID NO: 274 | SEQ ID NO: 308 | SEQ ID NO: 321 |
| SEQ ID NO: 113 | SEQ ID NO: 274 | SEQ ID NO: 309 | SEQ ID NO: 321 |
| SEQ ID NO: 114 | SEQ ID NO: 274 | SEQ ID NO: 302 | SEQ ID NO: 321 |
| SEQ ID NO: 115 | SEQ ID NO: 274 | SEQ ID NO: 397 | SEQ ID NO: 321 |
| SEQ ID NO: 116 | SEQ ID NO: 274 | SEQ ID NO: 397 | SEQ ID NO: 321 |
| SEQ ID NO: 117 | SEQ ID NO: 268 | SEQ ID NO: 296 | SEQ ID NO: 321 |
| SEQ ID NO: 118 | SEQ ID NO: 271 | SEQ ID NO: 296 | SEQ ID NO: 321 |
| SEQ ID NO: 119 | SEQ ID NO: 268 | SEQ ID NO: 302 | SEQ ID NO: 321 |
| SEQ ID NO: 120 | SEQ ID NO: 271 | SEQ ID NO: 302 | SEQ ID NO: 321 |
| SEQ ID NO: 121 | SEQ ID NO: 271 | SEQ ID NO: 310 | SEQ ID NO: 321 |

In some embodiments, an antibody, or antigen binding fragment thereof is provided, wherein the antibody or antibody fragment comprises the light chain variable region and corresponding light chain variable region CDRs provided in the following table.

| VL | VL CDR1 | VL CDR2 | VL CDR3 |
|---|---|---|---|
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 2 | SEQ ID NO: 322 | SEQ ID NO: 339 | SEQ ID NO: 351 |
| SEQ ID NO: 39 | SEQ ID NO: 323 | SEQ ID NO: 339 | SEQ ID NO: 351 |
| SEQ ID NO: 40 | SEQ ID NO: 324 | SEQ ID NO: 339 | SEQ ID NO: 351 |
| SEQ ID NO: 41 | SEQ ID NO: 325 | SEQ ID NO: 339 | SEQ ID NO: 351 |
| SEQ ID NO: 42 | SEQ ID NO: 326 | SEQ ID NO: 339 | SEQ ID NO: 351 |
| SEQ ID NO: 43 | SEQ ID NO: 327 | SEQ ID NO: 339 | SEQ ID NO: 351 |
| SEQ ID NO: 44 | SEQ ID NO: 328 | SEQ ID NO: 339 | SEQ ID NO: 351 |
| SEQ ID NO: 45 | SEQ ID NO: 329 | SEQ ID NO: 339 | SEQ ID NO: 351 |
| SEQ ID NO: 46 | SEQ ID NO: 330 | SEQ ID NO: 339 | SEQ ID NO: 351 |
| SEQ ID NO: 47 | SEQ ID NO: 331 | SEQ ID NO: 339 | SEQ ID NO: 351 |
| SEQ ID NO: 48 | SEQ ID NO: 332 | SEQ ID NO: 339 | SEQ ID NO: 351 |
| SEQ ID NO: 49 | SEQ ID NO: 333 | SEQ ID NO: 339 | SEQ ID NO: 351 |
| SEQ ID NO: 50 | SEQ ID NO: 322 | SEQ ID NO: 340 | SEQ ID NO: 351 |
| SEQ ID NO: 51 | SEQ ID NO: 322 | SEQ ID NO: 341 | SEQ ID NO: 351 |
| SEQ ID NO: 52 | SEQ ID NO: 322 | SEQ ID NO: 342 | SEQ ID NO: 351 |
| SEQ ID NO: 53 | SEQ ID NO: 322 | SEQ ID NO: 343 | SEQ ID NO: 351 |
| SEQ ID NO: 54 | SEQ ID NO: 322 | SEQ ID NO: 344 | SEQ ID NO: 351 |
| SEQ ID NO: 55 | SEQ ID NO: 322 | SEQ ID NO: 345 | SEQ ID NO: 351 |
| SEQ ID NO: 56 | SEQ ID NO: 322 | SEQ ID NO: 346 | SEQ ID NO: 351 |
| SEQ ID NO: 57 | SEQ ID NO: 322 | SEQ ID NO: 339 | SEQ ID NO: 352 |
| SEQ ID NO: 58 | SEQ ID NO: 322 | SEQ ID NO: 339 | SEQ ID NO: 353 |
| SEQ ID NO: 59 | SEQ ID NO: 322 | SEQ ID NO: 339 | SEQ ID NO: 354 |
| SEQ ID NO: 60 | SEQ ID NO: 322 | SEQ ID NO: 339 | SEQ ID NO: 355 |
| SEQ ID NO: 61 | SEQ ID NO: 322 | SEQ ID NO: 339 | SEQ ID NO: 356 |
| SEQ ID NO: 62 | SEQ ID NO: 322 | SEQ ID NO: 339 | SEQ ID NO: 357 |
| SEQ ID NO: 63 | SEQ ID NO: 322 | SEQ ID NO: 339 | SEQ ID NO: 358 |
| SEQ ID NO: 64 | SEQ ID NO: 322 | SEQ ID NO: 339 | SEQ ID NO: 359 |
| SEQ ID NO: 65 | SEQ ID NO: 322 | SEQ ID NO: 339 | SEQ ID NO: 360 |
| SEQ ID NO: 66 | SEQ ID NO: 322 | SEQ ID NO: 339 | SEQ ID NO: 361 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 8 | SEQ ID NO: 322 | SEQ ID NO: 339 | SEQ ID NO: 351 |
| SEQ ID NO: 89 | SEQ ID NO: 334 | SEQ ID NO: 339 | SEQ ID NO: 351 |
| SEQ ID NO: 90 | SEQ ID NO: 335 | SEQ ID NO: 339 | SEQ ID NO: 351 |
| SEQ ID NO: 91 | SEQ ID NO: 336 | SEQ ID NO: 339 | SEQ ID NO: 351 |
| SEQ ID NO: 92 | SEQ ID NO: 337 | SEQ ID NO: 339 | SEQ ID NO: 351 |
| SEQ ID NO: 93 | SEQ ID NO: 338 | SEQ ID NO: 339 | SEQ ID NO: 351 |
| SEQ ID NO: 94 | SEQ ID NO: 322 | SEQ ID NO: 347 | SEQ ID NO: 351 |
| SEQ ID NO: 95 | SEQ ID NO: 322 | SEQ ID NO: 348 | SEQ ID NO: 351 |
| SEQ ID NO: 96 | SEQ ID NO: 322 | SEQ ID NO: 349 | SEQ ID NO: 351 |
| SEQ ID NO: 97 | SEQ ID NO: 322 | SEQ ID NO: 350 | SEQ ID NO: 351 |
| SEQ ID NO: 98 | SEQ ID NO: 322 | SEQ ID NO: 339 | SEQ ID NO: 362 |
| SEQ ID NO: 100 | SEQ ID NO: 322 | SEQ ID NO: 348 | SEQ ID NO: 362 |
| SEQ ID NO: 100 | SEQ ID NO: 322 | SEQ ID NO: 348 | SEQ ID NO: 362 |
| SEQ ID NO: 100 | SEQ ID NO: 322 | SEQ ID NO: 348 | SEQ ID NO: 362 |
| SEQ ID NO: 100 | SEQ ID NO: 322 | SEQ ID NO: 348 | SEQ ID NO: 362 |
| SEQ ID NO: 100 | SEQ ID NO: 322 | SEQ ID NO: 348 | SEQ ID NO: 362 |
| SEQ ID NO: 100 | SEQ ID NO: 322 | SEQ ID NO: 348 | SEQ ID NO: 362 |
| SEQ ID NO: 100 | SEQ ID NO: 322 | SEQ ID NO: 348 | SEQ ID NO: 362 |
| SEQ ID NO: 100 | SEQ ID NO: 322 | SEQ ID NO: 348 | SEQ ID NO: 362 |
| SEQ ID NO: 100 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 109 | SEQ ID NO: 322 | SEQ ID NO: 348 | SEQ ID NO: 362 |
| SEQ ID NO: 122 | SEQ ID NO: 334 | SEQ ID NO: 348 | SEQ ID NO: 362 |
| SEQ ID NO: 122 | SEQ ID NO: 334 | SEQ ID NO: 348 | SEQ ID NO: 362 |
| SEQ ID NO: 122 | SEQ ID NO: 334 | SEQ ID NO: 348 | SEQ ID NO: 362 |
| SEQ ID NO: 122 | SEQ ID NO: 334 | SEQ ID NO: 348 | SEQ ID NO: 362 |
| SEQ ID NO: 122 | SEQ ID NO: 334 | SEQ ID NO: 348 | SEQ ID NO: 362 |
| SEQ ID NO: 122 | SEQ ID NO: 334 | SEQ ID NO: 348 | SEQ ID NO: 362 |
| SEQ ID NO: 122 | SEQ ID NO: 334 | SEQ ID NO: 348 | SEQ ID NO: 362 |
| SEQ ID NO: 122 | SEQ ID NO: 334 | SEQ ID NO: 348 | SEQ ID NO: 362 |
| SEQ ID NO: 122 | SEQ ID NO: 334 | SEQ ID NO: 348 | SEQ ID NO: 362 |
| SEQ ID NO: 122 | SEQ ID NO: 334 | SEQ ID NO: 348 | SEQ ID NO: 362 |
| SEQ ID NO: 122 | SEQ ID NO: 334 | SEQ ID NO: 348 | SEQ ID NO: 362 |
| SEQ ID NO: 122 | SEQ ID NO: 334 | SEQ ID NO: 348 | SEQ ID NO: 362 |

In some embodiments, an antibody, or antibody binding fragment thereof, comprises a heavy chain variable region (VH) sequence selected from the group consisting of SEQ ID NO: 1, 3, 4, 5, 6, 7, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 67, 68, 69, 70, 71, 72, 73, 74, 75, 76, 77, 78, 79, 80, 81, 82, 83, 84, 85, 86, 87, 88, 99, 101, 102, 103, 104, 105, 106, 107, 108, 110, 111, 112, 113, 114, 115, 116, 117, 118, 119, 120, and 121.

In some embodiments, an antibody, or antibody binding fragment thereof, comprises a light chain variable region (VL) sequence selected from the group consisting of SEQ ID NO: 2, 8, 39, 40, 41, 42, 43, 44, 45, 46, 47, 48, 49, 50, 51, 52, 53, 54, 55, 56, 57, 58, 59, 60, 61, 62, 63, 64, 65, 66, 89, 90, 91, 92, 93, 94, 95, 96, 97, 98, 100, 109, and 122.

In some embodiments, an antibody, or antibody binding fragment thereof, comprises a VH and VL pair comprising the amino acid sequence of SEQ ID NOs:123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, or a variant thereof.

In some embodiments, an antibody, or antibody binding fragment thereof, comprises a VH sequence of SEQ ID NO: 1 and a VL sequence of SEQ ID NO: 2. In some embodiments, an antibody, or antibody binding fragment thereof, comprises a VH sequence of SEQ ID NO: 3 and a VL sequence of SEQ ID NO: 2. In some embodiments, an antibody, or antibody binding fragment thereof, comprises a VH sequence of SEQ ID NO: 4 and a VL sequence of SEQ ID NO: 2. In some embodiments, an antibody, or antibody binding fragment thereof, comprises a VH sequence of SEQ ID NO: 5 and a VL sequence of SEQ ID NO: 2. In some embodiments, an antibody, or antibody binding fragment thereof, comprises a VH sequence of SEQ ID NO: 6 and a VL sequence of SEQ ID NO: 2. In some embodiments, an antibody, or antibody binding fragment thereof, comprises a VH sequence of SEQ ID NO: 7 and a VL sequence of SEQ ID NO: 2. In some embodiments, an antibody, or antibody binding fragment thereof, comprises a VH sequence of SEQ ID NO: 8 and a VL sequence of SEQ ID NO: 2. In some embodiments, an antibody, or antibody binding fragment thereof, comprises a VH sequence of SEQ ID NO: 9 and a VL sequence of SEQ ID NO: 2. In some embodiments, an antibody, or antibody binding fragment thereof, comprises a VH sequence of SEQ ID NO: 10 and a VL sequence of SEQ ID NO: 2. In some embodiments, an antibody, or antibody binding fragment thereof, comprises a VH sequence of SEQ ID NO: 11 and a VL sequence of SEQ ID NO: 2. In some embodiments, an antibody, or antibody binding fragment thereof, comprises a VH sequence of SEQ ID NO: 12 and a VL sequence of SEQ ID NO: 2. In some embodiments, an antibody, or antibody binding fragment thereof, comprises a VH sequence of SEQ ID NO: 13 and a VL sequence of SEQ ID NO: 2. In some embodiments, an antibody, or antibody binding fragment thereof, comprises a VH sequence of SEQ ID NO: 14 and a VL sequence of SEQ ID NO: 2. In some embodiments, an antibody, or antibody binding fragment thereof, comprises a VH sequence of SEQ ID NO: 15 and a VL sequence of SEQ ID NO: 2. In some embodiments, an antibody, or antibody binding fragment thereof, comprises a VH sequence of SEQ ID NO: 16 and a VL sequence of SEQ ID NO: 2. In some embodiments, an antibody, or antibody binding fragment thereof, comprises a VH sequence of SEQ ID NO: 17 and a VL sequence of SEQ ID NO: 2. In some embodiments, an antibody, or antibody binding fragment thereof, comprises a VH sequence of SEQ ID NO: 18 and a VL sequence of SEQ ID NO: 2. In some embodiments, an antibody, or antibody binding fragment thereof, comprises a VH sequence of SEQ ID NO: 19 and a VL sequence of SEQ ID NO: 2. In some embodiments, an antibody, or antibody binding fragment thereof, comprises a VH sequence of SEQ ID NO: 20 and a VL sequence of SEQ ID NO: 2. In some embodiments, an antibody, or antibody binding fragment thereof, comprises a VH sequence of SEQ ID NO: 21 and a VL sequence of SEQ ID NO: 2. In some embodiments, an antibody, or antibody binding fragment thereof, comprises a VH sequence of SEQ ID NO: 22 and a VL sequence of SEQ ID NO: 2. In some embodiments, an antibody, or antibody binding fragment thereof, comprises a VH sequence of SEQ ID NO: 23 and a VL sequence of SEQ ID NO: 2. In some embodiments, an antibody, or antibody binding fragment thereof, comprises a VH sequence of SEQ ID NO: 24 and a VL sequence of SEQ ID NO: 2. In some embodiments, an antibody, or antibody binding fragment thereof, comprises a VH sequence of SEQ ID NO: 25 and a VL sequence of SEQ ID NO: 2. In some embodiments, an antibody, or antibody binding fragment thereof, comprises a VH sequence of SEQ ID NO: 26 and a VL sequence of SEQ ID NO: 2. In some embodiments, an antibody, or antibody binding fragment thereof, comprises a VH sequence of SEQ ID NO: 27 and a VL sequence of SEQ ID NO: 2. In some embodiments, an antibody, or antibody binding fragment thereof, comprises a VH sequence of SEQ ID NO: 28 and a VL sequence of SEQ ID NO: 2. In some embodiments, an antibody, or antibody binding fragment thereof, comprises a VH sequence of SEQ ID NO: 29 and a VL sequence of SEQ ID NO: 2. In some embodiments, an antibody, or antibody binding fragment thereof, comprises a VH sequence of SEQ ID NO: 30 and a VL sequence of SEQ ID NO: 2. In some embodiments, an antibody, or antibody binding fragment thereof, comprises a VH sequence of SEQ ID NO: 31 and a VL sequence of SEQ ID NO: 2. In some embodiments, an antibody, or antibody binding fragment thereof, comprises a VH sequence of SEQ ID NO: 32 and a VL sequence of SEQ ID NO: 2. In some embodiments, an antibody, or antibody binding fragment thereof, comprises a VH sequence of SEQ ID NO: 33 and a VL sequence of SEQ ID NO: 2. In some embodiments, an antibody, or antibody binding fragment thereof, comprises a VH sequence of SEQ ID NO: 34 and a VL sequence of SEQ ID NO: 2. In some embodiments, an antibody, or antibody binding fragment thereof, comprises a VH sequence of SEQ ID NO: 35 and a VL sequence of SEQ ID NO: 2. In some embodiments, an antibody, or antibody binding fragment thereof, comprises a VH sequence of SEQ ID NO: 36 and a VL sequence of SEQ ID NO: 2. In some embodiments, an antibody, or antibody binding fragment thereof, comprises a VH sequence of SEQ ID NO: 37 and a VL sequence of SEQ ID NO: 2. In some embodiments, an antibody, or antibody binding fragment thereof, comprises a VH sequence of SEQ ID NO: 38 and a VL sequence of SEQ ID NO: 2.

In some embodiments, an antibody, or antibody binding fragment thereof, comprises a VH sequence of SEQ ID NO: 1 and a VL sequence of SEQ ID NO: 39. In some embodiments, an antibody, or antibody binding fragment thereof, comprises a VH sequence of SEQ ID NO: 1 and a VL sequence of SEQ ID NO: 40. In some embodiments, an antibody, or antibody binding fragment thereof, comprises a VH sequence of SEQ ID NO: 1 and a VL sequence of SEQ ID NO: 41. In some embodiments, an antibody, or antibody binding fragment thereof, comprises a VH sequence of SEQ ID NO: 1 and a VL sequence of SEQ ID NO: 42. In some embodiments, an antibody, or antibody binding fragment thereof, comprises a VH sequence of SEQ ID NO: 1 and a VL sequence of SEQ ID NO: 43. In some embodiments, an antibody, or antibody binding fragment thereof, comprises a VH sequence of SEQ ID NO: 1 and a VL sequence of SEQ ID NO: 44. In some embodiments, an antibody, or antibody binding fragment thereof, comprises a VH sequence of SEQ ID NO: 1 and a VL sequence of SEQ ID NO: 45. In some embodiments, an antibody, or antibody binding fragment thereof, comprises a VH sequence of SEQ ID NO: 1 and a VL sequence of SEQ ID NO: 46. In some embodiments, an antibody, or antibody binding fragment thereof, comprises a VH sequence of SEQ ID NO: 1 and a VL sequence of SEQ ID NO: 47. In some embodiments, an antibody, or antibody binding fragment thereof, comprises a VH sequence of SEQ ID NO: 1 and a VL sequence of SEQ ID NO: 48. In some embodiments, an antibody, or antibody binding fragment thereof, comprises a VH sequence of SEQ ID NO: 1 and a VL sequence of SEQ ID NO: 49. In some embodiments, an antibody, or antibody binding fragment thereof, comprises a VH sequence of SEQ ID NO: 1 and a VL sequence of SEQ ID NO: 50. In some embodiments, an antibody, or antibody binding fragment thereof, comprises a VH sequence of SEQ ID NO: 1 and a VL sequence of SEQ ID NO: 51. In some embodiments, an antibody, or antibody binding fragment thereof, comprises a VH sequence of SEQ ID NO: 1 and a VL sequence of SEQ ID NO: 52. In some embodiments, an antibody, or antibody binding fragment thereof, comprises a VH sequence of SEQ ID NO: 1 and a VL sequence of SEQ ID NO: 53. In some embodiments, an antibody, or antibody binding fragment thereof, comprises a VH sequence of SEQ ID NO: 1 and a VL sequence of SEQ ID NO: 54. In some embodiments, an antibody, or antibody binding fragment thereof, comprises a VH sequence of SEQ ID NO: 1 and a VL sequence of SEQ ID NO: 55. In some embodiments, an antibody, or antibody binding fragment thereof, comprises a VH sequence of SEQ ID NO: 1 and a VL sequence of SEQ ID NO: 56. In some embodiments, an antibody, or antibody binding fragment thereof, comprises a VH sequence of SEQ ID NO: 1 and a VL sequence of SEQ ID NO: 57. In some embodiments, an antibody, or antibody binding fragment thereof, comprises a VH sequence of SEQ ID NO: 1 and a VL sequence of SEQ ID NO: 58. In some embodiments, an antibody, or antibody binding fragment thereof, comprises a VH sequence of SEQ ID NO: 1 and a VL sequence of SEQ ID NO: 59. In some embodiments, an antibody, or antibody binding fragment thereof, comprises a VH sequence of SEQ ID NO: 1 and a VL sequence of SEQ ID NO: 60. In some embodiments, an antibody, or antibody binding fragment thereof, comprises a VH sequence of SEQ ID NO: 1 and a VL sequence of SEQ ID NO: 61. In some embodiments, an antibody, or antibody binding fragment thereof, comprises a VH sequence of SEQ ID NO: 1 and a VL sequence of SEQ ID NO: 62. In some embodiments, an antibody, or antibody binding fragment thereof, comprises a VH sequence of SEQ ID NO: 1 and a VL sequence of SEQ ID NO: 63. In some embodiments, an antibody, or antibody binding fragment thereof, comprises a VH sequence of SEQ ID NO: 1 and a VL sequence of SEQ ID NO: 64. In some embodiments, an antibody, or antibody binding fragment thereof, comprises a VH sequence of SEQ ID NO: 1 and a VL sequence of SEQ ID NO: 65. In some embodiments, an antibody, or antibody binding fragment thereof, comprises a VH sequence of SEQ ID NO: 1 and a VL sequence of SEQ ID NO: 66.

In some embodiments, an antibody, or antibody binding fragment thereof, comprises a VH sequence of SEQ ID NO: 67 and a VL sequence of SEQ ID NO: 8. In some embodiments, an antibody, or antibody binding fragment thereof, comprises a VH sequence of SEQ ID NO: 68 and a VL sequence of SEQ ID NO: 8. In some embodiments, an antibody, or antibody binding fragment thereof, comprises a VH sequence of SEQ ID NO: 69 and a VL sequence of SEQ ID NO: 8. In some embodiments, an antibody, or antibody binding fragment thereof, comprises a VH sequence of SEQ ID NO: 70 and a VL sequence of SEQ ID NO: 8. In some embodiments, an antibody, or antibody binding fragment thereof, comprises a VH sequence of SEQ ID NO: 71 and a VL sequence of SEQ ID NO: 8. In some embodiments, an antibody, or antibody binding fragment thereof, comprises a VH sequence of SEQ ID NO: 72 and a VL sequence of SEQ ID NO: 8. In some embodiments, an antibody, or antibody binding fragment thereof, comprises a VH sequence of SEQ ID NO: 73 and a VL sequence of SEQ ID NO: 8. In some embodiments, an antibody, or antibody binding fragment thereof, comprises a VH sequence of SEQ ID NO: 74 and a VL sequence of SEQ ID NO: 8. In some embodiments, an antibody, or antibody binding fragment thereof, comprises a VH sequence of SEQ ID NO: 75 and a VL sequence of SEQ ID NO: 8. In some embodiments, an antibody, or antibody binding fragment thereof, comprises a VH sequence of SEQ ID NO: 76 and a VL sequence of SEQ ID NO: 8. In some embodiments, an antibody, or antibody binding fragment thereof, comprises a VH sequence of SEQ ID NO: 77 and a VL sequence of SEQ ID NO: 8. In some embodiments, an antibody, or antibody binding fragment thereof, comprises a VH sequence of SEQ ID NO: 78 and a VL sequence of SEQ ID NO: 8. In some embodiments, an antibody, or antibody binding fragment thereof, comprises a VH sequence of SEQ ID NO: 79 and a VL sequence of SEQ ID NO: 8. In some embodiments, an antibody, or antibody binding fragment thereof, comprises a VH sequence of SEQ ID NO: 80 and a VL sequence of SEQ ID NO: 8. In some embodiments, an antibody, or antibody binding fragment thereof, comprises a VH sequence of SEQ ID NO: 81 and a VL sequence of SEQ ID NO: 8. In some embodiments, an antibody, or antibody binding fragment thereof, comprises a VH sequence of SEQ ID NO: 82 and a VL sequence of SEQ ID NO: 8. In some embodiments, an antibody, or antibody binding fragment thereof, comprises a VH sequence of SEQ ID NO: 83 and a VL sequence of SEQ ID NO: 8. In some embodiments, an antibody, or antibody binding fragment thereof, comprises a VH sequence of SEQ ID NO: 84 and a VL sequence of SEQ ID NO: 8. In some embodiments, an antibody, or antibody binding fragment thereof, comprises a VH sequence of SEQ ID NO: 85 and a VL sequence of SEQ ID NO: 8. In some embodiments, an antibody, or antibody binding fragment thereof, comprises a VH sequence of SEQ ID NO: 86 and a VL sequence of SEQ ID NO: 8. In some embodiments, an antibody, or antibody binding fragment thereof, comprises a VH sequence of SEQ ID NO: 87 and a VL sequence of SEQ ID NO: 8. In some embodiments, an antibody, or antibody binding fragment thereof, comprises a VH sequence of SEQ ID NO: 88 and a VL sequence of SEQ ID NO: 8.

In some embodiments, an antibody, or antibody binding fragment thereof, comprises a VH sequence of SEQ ID NO: 7 and a VL sequence of SEQ ID NO: 89. In some embodiments, an antibody, or antibody binding fragment thereof, comprises a VH sequence of SEQ ID NO: 7 and a VL sequence of SEQ ID NO: 90. In some embodiments, an antibody, or antibody binding fragment thereof, comprises a VH sequence of SEQ ID NO: 7 and a VL sequence of SEQ ID NO: 91. In some embodiments, an antibody, or antibody binding fragment thereof, comprises a VH sequence of SEQ ID NO: 7 and a VL sequence of SEQ ID NO: 92. In some embodiments, an antibody, or antibody binding fragment thereof, comprises a VH sequence of SEQ ID NO: 7 and a VL sequence of SEQ ID NO: 93. In some embodiments, an antibody, or antibody binding fragment thereof, comprises a VH sequence of SEQ ID NO: 7 and a VL sequence of SEQ ID NO: 94. In some embodiments, an antibody, or antibody binding fragment thereof, comprises a VH sequence of SEQ ID NO: 7 and a VL sequence of SEQ ID NO: 95. In some embodiments, an antibody, or antibody binding fragment thereof, comprises a VH sequence of SEQ ID NO: 7 and a VL sequence of SEQ ID NO: 96. In some embodiments, an antibody, or antibody binding fragment thereof, comprises a VH sequence of SEQ ID NO: 7 and a VL sequence of SEQ ID NO: 97. In some embodiments, an antibody, or antibody binding fragment thereof, comprises a VH sequence of SEQ ID NO: 7 and a VL sequence of SEQ ID NO: 98.

In some embodiments, an antibody, or antibody binding fragment thereof, comprises a VH sequence of SEQ ID NO: 99 and a VL sequence of SEQ ID NO: 100. In some embodiments, an antibody, or antibody binding fragment thereof, comprises a VH sequence of SEQ ID NO: 101 and a VL sequence of SEQ ID NO: 100. In some embodiments, an antibody, or antibody binding fragment thereof, comprises a VH sequence of SEQ ID NO: 102 and a VL sequence of SEQ ID NO: 100. In some embodiments, an antibody, or antibody binding fragment thereof, comprises a VH sequence of SEQ ID NO: 103 and a VL sequence of SEQ ID NO: 100. In some embodiments, an antibody, or antibody binding fragment thereof, comprises a VH sequence of SEQ ID NO: 104 and a VL sequence of SEQ ID NO: 100. In some embodiments, an antibody, or antibody binding fragment thereof, comprises a VH sequence of SEQ ID NO: 105 and a VL sequence of SEQ ID NO: 100. In some embodiments, an antibody, or antibody binding fragment thereof, comprises a VH sequence of SEQ ID NO: 106 and a VL sequence of SEQ ID NO: 100. In some embodiments, an antibody, or antibody binding fragment thereof, comprises a VH sequence of SEQ ID NO: 107 and a VL sequence of SEQ ID NO: 100. In some embodiments, an antibody, or antibody binding fragment thereof, comprises a VH sequence of SEQ ID NO: 108 and a VL sequence of SEQ ID NO: 100.

In some embodiments, an antibody, or antibody binding fragment thereof, comprises a VH sequence of SEQ ID NO: 99 and a VL sequence of SEQ ID NO: 109. In some embodiments, an antibody, or antibody binding fragment thereof, comprises a VH sequence of SEQ ID NO: 101 and a VL sequence of SEQ ID NO: 109. In some embodiments, an antibody, or antibody binding fragment thereof, comprises a VH sequence of SEQ ID NO: 102 and a VL sequence of SEQ ID NO: 109. In some embodiments, an antibody, or antibody binding fragment thereof, comprises a VH sequence of SEQ ID NO: 103 and a VL sequence of SEQ ID NO: 109. In some embodiments, an antibody, or antibody binding fragment thereof, comprises a VH sequence of SEQ ID NO: 104 and a VL sequence of SEQ ID NO: 109. In some embodiments, an antibody, or antibody binding fragment thereof, comprises a VH sequence of SEQ ID NO: 105 and a VL sequence of SEQ ID NO: 109. In some embodiments, an antibody, or antibody binding fragment thereof, comprises a VH sequence of SEQ ID NO: 106 and a VL sequence of SEQ ID NO: 109. In some embodiments, an antibody, or antibody binding fragment thereof, comprises a VH sequence of SEQ ID NO: 107 and a VL sequence of SEQ ID NO: 109. In some embodiments, an antibody, or antibody binding fragment thereof, comprises a VH sequence of SEQ ID NO: 108 and a VL sequence of SEQ ID NO: 109.

In some embodiments, an antibody, or antibody binding fragment thereof, comprises a VH sequence of SEQ ID NO: 110 and a VL sequence of SEQ ID NO: 109. In some embodiments, an antibody, or antibody binding fragment thereof, comprises a VH sequence of SEQ ID NO: 111 and a VL sequence of SEQ ID NO: 109. In some embodiments, an antibody, or antibody binding fragment thereof, comprises a VH sequence of SEQ ID NO: 112 and a VL sequence of SEQ ID NO: 109. In some embodiments, an antibody, or antibody binding fragment thereof, comprises a VH sequence of SEQ ID NO: 113 and a VL sequence of SEQ ID NO: 109. In some embodiments, an antibody, or antibody binding fragment thereof, comprises a VH sequence of SEQ ID NO: 114 and a VL sequence of SEQ ID NO: 109. In some embodiments, an antibody, or antibody binding fragment thereof, comprises a VH sequence of SEQ ID NO: 115 and a VL sequence of SEQ ID NO: 109. In some embodiments, an antibody, or antibody binding fragment thereof, comprises a VH sequence of SEQ ID NO: 116 and a VL sequence of SEQ ID NO: 109. In some embodiments, an antibody, or antibody binding fragment thereof, comprises a VH sequence of SEQ ID NO: 117 and a VL sequence of SEQ ID NO: 109. In some embodiments, an antibody, or antibody binding fragment thereof, comprises a VH sequence of SEQ ID NO: 118 and a VL sequence of SEQ ID NO: 109. In some embodiments, an antibody, or antibody binding fragment thereof, comprises a VH sequence of SEQ ID NO: 119 and a VL sequence of SEQ ID NO: 109. In some embodiments, an antibody, or antibody binding fragment thereof, comprises a VH sequence of SEQ ID NO: 120 and a VL sequence of SEQ ID NO: 109. In some embodiments, an antibody, or antibody binding fragment thereof, comprises a VH sequence of SEQ ID NO: 121 and a VL sequence of SEQ ID NO: 109.

In some embodiments, an antibody, or antibody binding fragment thereof, comprises a VH sequence of SEQ ID NO: 110 and a VL sequence of SEQ ID NO: 122. In some embodiments, an antibody, or antibody binding fragment thereof, comprises a VH sequence of SEQ ID NO: 111 and a VL sequence of SEQ ID NO: 122. In some embodiments, an antibody, or antibody binding fragment thereof, comprises a VH sequence of SEQ ID NO: 112 and a VL sequence of SEQ ID NO: 122. In some embodiments, an antibody, or antibody binding fragment thereof, comprises a VH sequence of SEQ ID NO: 113 and a VL sequence of SEQ ID NO: 122. In some embodiments, an antibody, or antibody binding fragment thereof, comprises a VH sequence of SEQ ID NO: 114 and a VL sequence of SEQ ID NO: 122. In some embodiments, an antibody, or antibody binding fragment thereof, comprises a VH sequence of SEQ ID NO: 115 and a VL sequence of SEQ ID NO: 122. In some embodiments, an antibody, or antibody binding fragment thereof, comprises a VH sequence of SEQ ID NO: 116 and a VL sequence of SEQ ID NO: 122. In some embodiments, an antibody, or antibody binding fragment thereof, comprises a VH sequence of SEQ ID NO: 117 and a VL sequence of SEQ ID NO: 122. In some embodiments, an antibody, or antibody binding fragment thereof, comprises a VH sequence of SEQ ID NO: 118 and a VL sequence of SEQ ID NO: 122. In some embodiments, an antibody, or antibody binding fragment thereof, comprises a VH sequence of SEQ ID NO: 119 and a VL sequence of SEQ ID NO: 122. In some embodiments, an antibody, or antibody binding fragment thereof, comprises a VH sequence of SEQ ID NO: 120 and a VL sequence of SEQ ID NO: 122. In some embodiments, an antibody, or antibody binding fragment thereof, comprises a VH sequence of SEQ ID NO: 121 and a VL sequence of SEQ ID NO: 122.

In some embodiments, an antibody, or antibody binding fragment thereof, comprises a VH complementarity-determining region (CDR) sequence selected from the group consisting of SEQ ID NO: 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, and 397.

In some embodiments, an antibody, or antibody binding fragment thereof, comprises a VL CDR sequence selected from the group consisting of SEQ ID NO: 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, and 362.

In some embodiments, an antibody, or antibody binding fragment thereof, comprises a heavy chain variable region comprising heavy chain CDRl, CDR2, and CDR3 sequences, wherein the heavy chain CDR1 sequence has the amino acid sequence of SEQ ID NO: 263, 264, 265, 266, 267, 268, 269, 270, 271, 272, 273, 274, 275, or 276; the heavy chain CDR2 has the amino acid sequence of SEQ ID NO: 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, or 397; and the heavy chain CDR3 sequence has the amino acid sequence of SEQ ID NO: 311, 312, 313, 314, 315, 316, 317, 318, 319, 320, or 321; or variants of any of the foregoing.

In some embodiments, an antibody, or antibody binding fragment thereof, comprises a light chain variable region comprising light chain CDR1, CDR2, and CDR3 sequences, wherein the light chain CDR1 sequence has the amino acid sequence SEQ ID NO: 322, 323, 324, 325, 326, 327, 328, 329, 330, 331, 332, 333, 334, 335, 336, 337, or 338; the light chain CDR2 sequence has the amino acid sequence of SEQ ID NO: 339, 340, 341, 342, 343, 344, 345, 346, 347, 348, 349, or 350; and the light chain CDR3 sequence has the amino acid sequence of SEQ ID NO: 351, 352, 353, 354, 355, 356, 357, 358, 359, 360, 361, or 362; or variants of any of the foregoing.

In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 264, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 265, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 266, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 267, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 351.

In some embodiments, an antibody, or antibody binding fragment thereof, comprises a heavy chain variable region with a CDR1 of SEQ ID NO: 263, a CDR2 of SEQ ID NO: 278, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 279, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 280, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 281, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 282, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 283, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 284, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 285, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 286, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 287, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 288, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 289, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 290, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 291, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 292, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 293, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 294, and a CDR3 of SEQ ID NO: 311; and a light chain variable region with a CDR1 of SEQ ID NO: 322, a CDR2 of SEQ ID NO: 339, and a CDR3 of SEQ ID NO: 351.

In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 312; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 313; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 314; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 315; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 316; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 317; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 318; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 319; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 320; and a light chain variable region with a CDR1 of SEQ ID NO: 322, a CDR2 of SEQ ID NO: 339, and a CDR3 of SEQ ID NO: 351.

In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 323, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 324, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 325, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 326, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 327, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 328, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 329, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 330, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 331, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 332, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 333, a CDR2 of SEQ ID NO: 339, and a CDR3 of SEQ ID NO: 351.

In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 340, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 341, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 342, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 343, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 344, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 345, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 346, and a CDR3 of SEQ ID NO: 351.

In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 352. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 353. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 354. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 355. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 356. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 357. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 358. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 359. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 360. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 361.

In some embodiments, an antibody, or antibody binding fragment thereof, comprises a heavy chain variable region with a CDR1 of SEQ ID NO: 268, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 269, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 270, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 271, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 272, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 273, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 274, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 275, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 276, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 351.

In some embodiments, an antibody, or antibody binding fragment thereof, comprises a heavy chain variable region with a CDR1 of SEQ ID NO: 263, a CDR2 of SEQ ID NO: 295, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 296, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 297, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 298, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 299, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 300, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 301, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 302, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 303, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 304, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 305, and a CDR3 of SEQ ID NO: 311; and a light chain variable region with a CDR1 of SEQ ID NO: 322, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 306, and a CDR3 of SEQ ID NO: 311; and a light chain variable region with a CDR1 of SEQ ID NO: 322, a CDR2 of SEQ ID NO: 339, and a CDR3 of SEQ ID NO: 351.

In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 321; and a light chain variable region with a CDR1 of SEQ ID NO: 322, a CDR2 of SEQ ID NO: 339, and a CDR3 of SEQ ID NO: 351.

In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 334, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 335, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 336, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 337, a CDR2 of SEQ ID NO: 339, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 338, a CDR2 of SEQ ID NO: 339, and a CDR3 of SEQ ID NO: 351.

In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 347, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 348, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 349, and a CDR3 of SEQ ID NO: 351. In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 350, and a CDR3 of SEQ ID NO: 351.

In some embodiments, an antibody, or antibody binding fragment thereof, comprises a heavy chain variable region with a CDR1 of SEQ ID NO: 263, a CDR2 of SEQ ID NO: 277, and a CDR3 of SEQ ID NO: 311; and a light chain variable region with a CDR1 of SEQ ID NO: 322, a CDR2 of SEQ ID NO: 339, and a CDR3 of SEQ ID NO: 362.

In some embodiments, an antibody, or antibody binding fragment thereof, comprises a heavy chain variable region with a CDR1 of SEQ ID NO: 274, a CDR2 of SEQ ID NO: 296, and a CDR3 of SEQ ID NO: 321; and a light chain variable region with a CDR1 of SEQ ID NO: 322, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 274, a CDR2 of SEQ ID NO: 298, and a CDR3 of SEQ ID NO: 321; and a light chain variable region with a CDR1 of SEQ ID NO: 322, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 274, a CDR2 of SEQ ID NO: 300, and a CDR3 of SEQ ID NO: 321; and a light chain variable region with a CDR1 of SEQ ID NO: 322, a CDR2 of SEQ ID NO: 348, and a CDR3 of SEQ ID NO: 362.

In some embodiments, an antibody, or antibody binding fragment thereof, comprises a heavy chain variable region with a CDR1 of SEQ ID NO: 271, a CDR2 of SEQ ID NO: 296, and a CDR3 of SEQ ID NO: 321; and a light chain variable region with a CDR1 of SEQ ID NO: 322, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 271, a CDR2 of SEQ ID NO: 298, and a CDR3 of SEQ ID NO: 321; and a light chain variable region with a CDR1 of SEQ ID NO: 322, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 271, a CDR2 of SEQ ID NO: 300, and a CDR3 of SEQ ID NO: 321; and a light chain variable region with a CDR1 of SEQ ID NO: 322, a CDR2 of SEQ ID NO: 348, and a CDR3 of SEQ ID NO: 362.

In some embodiments, an antibody, or antibody binding fragment thereof, comprises a heavy chain variable region with a CDR1 of SEQ ID NO: 272, a CDR2 of SEQ ID NO: 296, and a CDR3 of SEQ ID NO: 321; and a light chain variable region with a CDR1 of SEQ ID NO: 322, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 272, a CDR2 of SEQ ID NO: 298, and a CDR3 of SEQ ID NO: 321; and a light chain variable region with a CDR1 of SEQ ID NO: 322, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 272, a CDR2 of SEQ ID NO: 300, and a CDR3 of SEQ ID NO: 321; and a light chain variable region with a CDR1 of SEQ ID NO: 322, a CDR2 of SEQ ID NO: 348, and a CDR3 of SEQ ID NO: 362.

In some embodiments, an antibody, or antibody binding fragment thereof, comprises a heavy chain variable region with a CDR1 of SEQ ID NO: 274, a CDR2 of SEQ ID NO: 307, and a CDR3 of SEQ ID NO: 321; and a light chain variable region with a CDR1 of SEQ ID NO: 322, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 274, a CDR2 of SEQ ID NO: 308, and a CDR3 of SEQ ID NO: 321; and a light chain variable region with a CDR1 of SEQ ID NO: 322, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 274, a CDR2 of SEQ ID NO: 309, and a CDR3 of SEQ ID NO: 321; and a light chain variable region with a CDR1 of SEQ ID NO: 322, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 274, a CDR2 of SEQ ID NO: 302, and a CDR3 of SEQ ID NO: 321; and a light chain variable region with a CDR1 of SEQ ID NO: 322, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 274, a CDR2 of SEQ ID NO: 397, and a CDR3 of SEQ ID NO: 321; and a light chain variable region with a CDR1 of SEQ ID NO: 322, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 268, a CDR2 of SEQ ID NO: 296, and a CDR3 of SEQ ID NO: 321; and a light chain variable region with a CDR1 of SEQ ID NO: 322, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 271, a CDR2 of SEQ ID NO: 296, and a CDR3 of SEQ ID NO: 321; and a light chain variable region with a CDR1 of SEQ ID NO: 322, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 268, a CDR2 of SEQ ID NO: 302, and a CDR3 of SEQ ID NO: 321; and a light chain variable region with a CDR1 of SEQ ID NO: 322, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 271, a CDR2 of SEQ ID NO: 302, and a CDR3 of SEQ ID NO: 321; and a light chain variable region with a CDR1 of SEQ ID NO: 322, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 271, a CDR2 of SEQ ID NO: 310, and a CDR3 of SEQ ID NO: 321; and a light chain variable region with a CDR1 of SEQ ID NO: 322, a CDR2 of SEQ ID NO: 348, and a CDR3 of SEQ ID NO: 362.

In some embodiments, an antibody, or antibody binding fragment thereof, comprises a heavy chain variable region with a CDR1 of SEQ ID NO: 274, a CDR2 of SEQ ID NO: 307, and a CDR3 of SEQ ID NO: 321; and a light chain variable region with a CDR1 of SEQ ID NO: 334, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 274, a CDR2 of SEQ ID NO: 308, and a CDR3 of SEQ ID NO: 321; and a light chain variable region with a CDR1 of SEQ ID NO: 334, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 274, a CDR2 of SEQ ID NO: 309, and a CDR3 of SEQ ID NO: 321; and a light chain variable region with a CDR1 of SEQ ID NO: 334, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 274, a CDR2 of SEQ ID NO: 302, and a CDR3 of SEQ ID NO: 321; and a light chain variable region with a CDR1 of SEQ ID NO: 334, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 274, a CDR2 of SEQ ID NO: 397, and a CDR3 of SEQ ID NO: 321; and a light chain variable region with a CDR1 of SEQ ID NO: 334, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 268, a CDR2 of SEQ ID NO: 296, and a CDR3 of SEQ ID NO: 321; and a light chain variable region with a CDR1 of SEQ ID NO: 334, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 271, a CDR2 of SEQ ID NO: 296, and a CDR3 of SEQ ID NO: 321; and a light chain variable region with a CDR1 of SEQ ID NO: 334, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 268, a CDR2 of SEQ ID NO: 302, and a CDR3 of SEQ ID NO: 321; and a light chain variable region with a CDR1 of SEQ ID NO: 334, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 271, a CDR2 of SEQ ID NO: 302, and a CDR3 of SEQ ID NO: 321; and a light chain variable region with a CDR1 of SEQ ID NO: 334, a CDR2 of SEQ ID NO: 348, and a CDR3 of SEQ ID NO: 362. In some embodiments, an antibody, or antibody binding fragment thereof, comprises a heavy chain variable region with a CDR1 of SEQ ID NO: 271, a CDR2 of SEQ ID NO: 310, and a CDR3 of SEQ ID NO: 321; and a light chain variable region with a CDR1 of SEQ ID NO: 334, a CDR2 of SEQ ID NO: 348, and a CDR3 of SEQ ID NO: 362.

In some embodiments, the antibody, or antigen binding fragment thereof, comprises a VH peptide comprising an amino acid sequence as set forth in SEQ ID NOs: 1, 3, 4, 5, 6, 7, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 67, 68, 69, 70, 71, 72, 73, 74, 75, 76, 77, 78, 79, 80, 81, 82, 83, 84, 85, 86, 87, 88, 99, 101, 102, 103, 104, 105, 106, 107, 108, 110, 111, 112, 113, 114, 115, 116, 117, 118, 119, 120, or 121, or any variant thereof.

In some embodiments, the antibody, or antigen binding fragment thereof, comprises a VL peptide comprising an amino acid sequence as set forth in SEQ ID NOs: 2, 8, 39, 40, 41, 42, 43, 44, 45, 46, 47, 48, 49, 50, 51, 52, 53, 54, 55, 56, 57, 58, 59, 60, 61, 62, 63, 64, 65, 66, 89, 90, 91, 92, 93, 94, 95, 96, 97, 98, 100, 109, or 122, or any variant thereof.

In some embodiments, the antibody, or antigen binding fragment thereof, comprises a sequence that is at least at least 85%, 90%, 91%, 92%, 93%, 94%, 95%, 96%, 97%, 98%, or 99% homology to a sequence of SEQ ID NOs: 1-122. In some embodiments, the antibody, or antigen binding fragment thereof, comprises a sequence that is at least at least 85%, 90%, 91%, 92%, 93%, 94%, 95%, 96%, 97%, 98%, or 99% homology to a sequence of SEQ ID NOs: 123-262. In some embodiments, the antibody, or antigen binding fragment thereof, comprises a sequence that is at least at least 85%, 90%, 91%, 92%, 93%, 94%, 95%, 96%, 97%, 98%, or 99% homology to a sequence of SEQ ID NOs: 263-362, and 397.

In addition to these specific combinations any of the VH peptides and the V_{L} peptides can be combined with one another.

In addition to these specific combinations any of the HC peptides and the LC peptides can be combined with one another.

In some embodiments, the antibody comprises a sequence, or antigen binding fragment of ATCC clone PTA-7444. The sequence of the antibody produced by ATCC clone PTA-7444 is hereby incorporated by reference in its entirety, which includes the antigen binding fragments thereof.

Additionally, as provided for herein, the antibodies can be multi-specific antibodies, in that the antibodies have multiple binding regions that target different proteins or the same protein at different epitopes. In some embodiments, the antibody is a bi-specific antibody.

As provided for herein, the different peptides (VH or V_{L}) described herein can be linked with a peptide linker or not linked with a peptide linker and instead for a contiguous sequence. In some embodiments, the peptide linker comprises a sequence of: (GGGGS)ₙ (SEQ ID NO: 375); (GGGGA)ₙ (SEQ ID NO: 376), or any combination thereof, wherein each n is independently 1-5. The linked peptide format can be represented by a formula of V_{H}-Z-V_{L} or V_{L}-Z-V_{H}, wherein Z is the peptide linker. In some embodiments, Z is (GGGGS)ₙ (SEQ ID NO: 375); (GGGGA)ₙ (SEQ ID NO: 376), or any combination thereof, wherein each n is independently 1-5.

As provided for herein, the antibodies, or antigen binding fragments thereof can be variants of the sequences.

Other examples of antibodies include, but are not limited to, those provided in US20160096894A1, EP1399483B1, EP2194067B1, US20040202651A1, US20110229933A1, U.S. Pat. No. 8,137,933B2, U.S. Pat. No. 8,951,790B2, US20190270820A1, U.S. Pat. No. 7,572,897B2, US20090275126A1, EP1959014B1, US20080014203A1, US20080226635A1, US20120076778A1, US20190153071A1, WO2011161119A1, U.S. Ser. No. 10/611,825B2, US20120237507A1, EP2681240B1, U.S. Pat. No. 9,982,036B2, US20180312573A1, EP2681239B1, US20160151487A1, US20190225696A1, WO2017011773A2, US20200023076A1, US20190153471A1, US20190194713A1, WO2020006486A1, US20080112888A1, US20150168424A1, EP2032989B2, U.S. Pat. No. 9,045,536B2, each of which is hereby incorporated by reference in its entirety. Other examples of antibodies include, but are not limited to, those provided in U.S. Pat. No. 8,153,121B2, EP1469879B1, WO2016064716A1, US20190270820A1, US20180280527A1, US20190225696A1, U.S. Pat. No. 7,998,681B2, US20040202651A1, US20050136063A1, US20090285824A1, US20150274829A1, EP2322550B1, US20060286103A1, US20070071675A1, US20100047239A1, US20130004416A1, US20080112888A1, US20150168424A1, US20100143340A1, US20110014117A1, US20100260668A1, US20100074900A1, US20150017168A1, US20110044980A1, US20130330323A1, US20120263722A1, US20120201746A1, U.S. Ser. No. 10/519,245B2, US20180243432A1, US20170218091A1, US20200115460A1, US20100104645A1, US20120065380A1, EP2970433B1, US20160289341A1, US20160289343A1, US20190293656A1, each of which is hereby incorporated by reference in its entirety.

### Pharmaceutical Compositions

In some embodiments, to prepare pharmaceutical or sterile compositions of the anti-IGF-1R antibodies or other proteins provided herein, the antibody or antigen binding fragment thereof or other proteins provided herein are admixed with a pharmaceutically acceptable carrier or excipient. See, e.g., Remington's Pharmaceutical Sciences and U.S. Pharmacopeia: National Formulary, Mack Publishing Company, Easton, PA (1984).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY). In some embodiments embodiment, the antibodies are diluted to an appropriate concentration in a sodium acetate solution pH 5-6, and NaCl or sucrose is added for tonicity. Additional agents, such as polysorbate 20 or polysorbate 80, may be added to enhance stability.

Toxicity and therapeutic efficacy of the antibody compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, antibodies exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

In some embodiments, a composition of the invention is administered to a subject in accordance with the Physicians' Desk Reference 2003 (Thomson Healthcare; 57th edition (Nov. 1, 2002)).

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In some embodiments, the antibody or antigen binding fragment thereof can be administered by an invasive route such as by injection. In some embodiments, the antibodies or antigen binding fragment thereof, or pharmaceutical composition thereof, is administered intravenously, subcutaneously, intramuscularly, intraarterially, intra-particularly (e.g. in arthritis joints), or by inhalation, aerosol delivery. Administration by non-invasive routes (e.g., orally; for example, in a pill, capsule or tablet) is also within the scope of the present embodiments.

In some embodiments, the antibody or antigen binding fragment thereof can be administered directly to the eye, the anterior chamber of the eye, the vitreous chamber of the eye, the suprachoroidal space, or the retro-orbital sinus. In some embodiments, administration to the eye, the anterior chamber of the eye, the vitreous chamber of the eye, the suprachoroidal space, or the retro-orbital sinus is via an injection. In some embodiments, the injection is an intravitreal injection, intraorbital injection, retro-orbital injection, suprachoroidal injection, or intracameral injection. In some embodiments, the injection is an intravitreal injection. In some embodiments, the injection is an, intraorbital injection. In some embodiments, the injection is a retro-orbital injection. In some embodiments, the injection is a suprachoroidal injection. In some embodiments, the injection is an intracameral injection.

In some embodiments, the anti-IGF-1R antibody, or antigen binding fragment thereof, is administered in combination with at least one additional therapeutic agent, such as, but not limited to any therapeutic used to treat thyroid eye disease. For example, in some embodiments, the anti-IGF-1R antibody, or antigen binding fragment thereof, is administered in combination with at least one additional therapeutic agent, such as, but not limited to a therapeutic used to treat thyroid eye disease or a condition related to the same. Examples of such treatments and therapeutics include, but are not limited to anti-thyroid medications, diabetes medications, beta-blockers, propylthiouracil, methimazole, propranolol, atenolol, metoprolol, nadolol, corticosteroids, metformin, sulfonylureas, meglitinides, thiazolidinediones, DPP-4 inhibitors, GLP-1 receptor agonists, SGLT2 inhibitors, regular insulin, insulin aspart, insulin glulisine, insulin lispro, insulin isophane, insulin degludec, insulin detemir, insulin glargine, acerbose, miglitol, acebutolol, atenolol, betaxolol, bisoprolol, cartelol, carvedilol, esmolol, labetalol, metoprolol, nadolol, nebivolol, penbutolol, pindolol, propranolol, sotalol, timolol, tomolol ophthalmic solution, sitagliptin, saxagliptin, linagliptin, alogliptin, dulaglutide, exenatide, semaglutide, liraglutide, lixisenatide, canagliflozin, dapagliflozin, empagliflozin, or any combination thereof.

Compositions can be administered with medical devices known in the art. For example, a pharmaceutical composition of the invention can be administered by injection with a hypodermic needle, including, e.g., a prefilled syringe or autoinjector.

The pharmaceutical compositions may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

The pharmaceutical compositions may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternately, one may administer the antibody in a local rather than systemic manner, for example, via injection of the antibody directly into an arthritic joint or pathogen-induced lesion characterized by immunopathology, often in a depot or sustained release formulation. Furthermore, one may administer the antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

The administration regimen depends on several factors, including the serum or tissue turnover rate of the therapeutic antibody, the level of symptoms, the immunogenicity of the therapeutic antibody, and the accessibility of the target cells in the biological matrix. Preferably, the administration regimen delivers sufficient therapeutic antibody to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert, et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602).

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent. In the case of human subjects, for example, chimeric, humanized and fully human antibodies are may be desirable.

Antibodies or antigen binding fragments thereof can be provided by continuous infusion, or by doses administered, e.g., daily, 1-7 times per week, weekly, bi-weekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. In some embodiments, the antibody is administered every three weeks, every four weeks, every five weeks, every six weeks, every seven weeks, or every eight weeks. In some embodiments, the antibody is administered every four weeks. In some embodiments, the antibody is administered every five weeks. In some embodiments, the antibody is administered every seven weeks. In some embodiments, the antibody is administered every six weeks. In some embodiments, the antibody is administered every eight weeks. In some embodiments, the antibody is administered for at least 21-52 weeks or longer. In some embodiments, the antibody is administered on such a schedule for at least 21 weeks. In some embodiments, the antibody is administered on such a schedule for at least 24 weeks. In some embodiments, the antibody is administered on such a schedule for at least 32 weeks. In some embodiments, the antibody is administered on such a schedule for at least 36 weeks. In some embodiments, the antibody is administered on such a schedule for at least 40 weeks. In some embodiments, the antibody is administered on such a schedule for at least 42 weeks. In some embodiments, the antibody is administered (e.g. infusion or subcutaneous injection) once. In some embodiments, the antibody is administered (e.g. infusion or subcutaneous injection) twice. In some embodiments, the antibody is administered (e.g. infusion or subcutaneous injection) three times. In some embodiments, the antibody is administered (e.g. infusion or subcutaneous injection) four times. In some embodiments, the antibody is administered (e.g. infusion or subcutaneous injection) five times. In some embodiments, the antibody is administered (e.g. infusion or subcutaneous injection) six times. In some embodiments, the antibody is administered (e.g. infusion or subcutaneous injection) seven times. In some embodiments, the antibody is administered (e.g. infusion or subcutaneous injection) eight times. In some embodiments, the antibody is administered (e.g. infusion or subcutaneous injection) nine times. In some embodiments, the antibody is administered (e.g. infusion or subcutaneous injection) 10 times. In some embodiments, the antibody is administered (e.g. infusion or subcutaneous injection) 11 times. In some embodiments, the antibody is administered (e.g. infusion or subcutaneous injection) 12 times. In some embodiments, the antibody is administered (e.g. infusion or subcutaneous injection) 13 times. In some embodiments, the antibody is administered (e.g. infusion or subcutaneous injection) 14 times. In some embodiments, the antibody is administered (e.g. infusion or subcutaneous injection) 15 times. In some embodiments, the antibody is administered (e.g. infusion or subcutaneous injection) 16 times. In some embodiments, the antibody is administered (e.g. infusion or subcutaneous injection) 17 times. In some embodiments, the antibody is administered (e.g. infusion or subcutaneous injection) 18 times. In some embodiments, the antibody is administered (e.g. infusion or subcutaneous injection) 19 times. In some embodiments, the antibody is administered (e.g. infusion or subcutaneous injection) 20 times. When the antibody is administered more than once it can be administered according to a schedule, such as the schedules provided for herein.

A total weekly dose can be as provided for herein. In some embodiments, the total weekly dose is at least 0.05 μg/kg body weight, more generally at least 0.2 μg/kg, 0.5 g/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more (see, e.g., Yang, et al. (2003) New Engl. J. Med. 349:427-434; Herold, et al. (2002) New Engl. J. Med. 346:1692-1698; Liu, et al. (1999) J. Neurol. Neurosurg. Psych. 67:451-456; Portielji, et al. (20003) Cancer Immunol. Immunother. 52:133-144). Doses may also be provided to achieve a pre-determined target concentration of the antibody in the subject's serum, such as 0.1, 0.3, 1, 3, 10, 30, 100, 300 μg/ml or more.

In some embodiments, the antibody has a serum concentration in the subject of at least, or about, 10 μg/ml or 20 μg/ml or 50 μg/ml, 70 μg/ml, 75 μg/ml, 80 μg/ml, 85 μg/ml, 90 μg/ml, 95 μg/ml, 100 μg/ml, or 105 μg/ml at least 1, 2, or 3 weeks after administration.

In some embodiments, a dose of 20 mg/kg IV is administered In some embodiments, a dosing is used to provide a Cmin of 133 μg/mL after about 5 weeks. In some embodiments, the dose of the antibody that is administered that provides a Cmin of 102 μg/mL after 6 weeks. In some embodiments, the dose of the antibody is as provided for herein, such as 10 mg/mg as a loading dose with subsequent doses being the same or lower. In some embodiments, the antibody is administered as provided for herein at a dose to achieve a Cmin of at least, or about, 100 μg/mL.

As used herein, “inhibit” or “treat” or “treatment” includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom.

As used herein, the terms “therapeutically effective amount”, “therapeutically effective dose” and “effective amount” refer to an amount of the antibody, or antigen binding fragment thereof, that, when administered alone or in combination with an additional therapeutic agent to a cell, tissue, or subject, is effective to cause a measurable improvement in one or more symptoms of a disease or condition or the progression of such disease or condition. A therapeutically effective dose further refers to that amount of the binding compound sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity. In some embodiments, an amount is a therapeutically effective amount if it is an amount that can be used to treat or ameliorate a condition as provided for herein.

The term “subject” as used throughout includes any organism, such as an animal, including a mammal (e.g., rat, mouse, dog, cat, rabbit) and, for example, a human. A subject can be also be referred to as a patient. In some embodiments, the subject is a subject in need thereof. A subject that is “in need thereof” refers to a subject that has been identified as requiring treatment for the condition that is to be treated and is treated with the specific intent of treating such condition. The conditions can be, for example, any of the conditions described herein.

Whereas, an isolated antibody binds an epitope on a IGF-1R protein, or other protein described herein, and displays in vitro and/or in vivo IGF-1R inhibiting or therapeutic activities, the antibodies or antigen binding fragments thereof, capable of inhibiting IGF-1R function, are suitable both as therapeutic agents for treating IGF-1R-associated conditions in humans and animals. These conditions include thyroid eye disease. According, methods of treating such conditions are also provided, wherein the method comprises administering an antibody, or antigen binding fragment thereof, to the subject with such a condition.

In some embodiments, the methods comprise administering a therapeutically or prophylactically effective amount of one or more monoclonal antibodies or antigen binding fragments of the antibodies described herein to a susceptible subject or to one exhibiting a condition in which IGF-1R is known or suspected to have caused the pathology observed. Any active form of the antibody can be administered, including, but not limited to scFV, Fab and F(ab′)2 fragments and other forms of antibodies provided for herein.

As used herein, a IGF-1R associated pathology refers to conditions that are caused by the modulation of IGF-1R. These conditions include, but are not limited to, thyroid eye disease and other conditions provided for herein.

In some embodiments, the antibodies used are compatible with the recipient species such that the immune response to the MAbs does not result in an unacceptably short circulating half-life or induce an immune response to the MAbs in the subject.

Treatment of individuals may comprise the administration of a therapeutically effective amount of the antibodies described herein. The antibodies can be provided in a kit, such as those provided herein. The antibodies can be used or administered alone or in admixture with another therapeutic, analgesic, or diagnostic agent, such as provided for herein. In providing a patient with an antibody, or fragment thereof, capable of binding to IGF-1R, or an antibody capable of protecting against IGF-1R, pathology in a recipient patient, the dosage of administered agent will vary depending upon such factors as the patient's age, weight, height, sex, general medical condition, previous medical history, etc.

An antibody, capable treating a condition associated with IGF-1R activity or use to treat a IGF-1R related pathology, is intended to be provided to subjects in an amount sufficient to affect a reduction, resolution, or amelioration in the IGF-1R related symptom or pathology. Such a pathology includes, thyroid eye disease and the like

Accordingly, in some embodiments, methods of treating a subject with a IGF-1R mediated disorder are provided. In some embodiments, the method comprises administering a pharmaceutical composition comprising an antibody, or antigen binding fragment thereof, as provided herein. In some embodiments, the disorder is thyroid eye disease. As provided for herein, the antibodies, or antigen binding fragments thereof, can be administered with other therapeutics. These can be administered simultaneously or sequentially.

In some embodiments, the antibodies, or antigen binding fragments thereof, may be used to treat thyroid eye disease. In some embodiments, the antibodies, or antigen binding fragments thereof, may be used to treating or reduce the severity of, thyroid-associated ophthalmopathy (TAO), or a symptom thereof.

In some embodiments, methods or uses are provided to reduce proptosis in an eye in a subject with thyroid-associated ophthalmopathy (TAO).

In some embodiments, the subject is a subject how has previously been treated with a different antibody than those provided herein.

In some embodiments, methods or uses are provided to Clinical Activity Score (CAS) in subject who has or is suspected of having thyroid-associated ophthalmopathy (TAO).

In some embodiments, methods or uses are provided to reduce proptosis by at least 2 mm and b) reducing the clinical activity score (CAS) in a subject with thyroid-associated ophthalmopathy (TAO).

As used herein, the term Clinical Activity Score (CAS) refers to the protocol described and scored according to Table 2. According to this protocol, one point is given for the presence of each of the parameters assessed in the Table below. The sum of all points defines clinical activity and provides the CAS, where 0 or 1 constitutes inactive disease and 7 severe active ophthalmopathy.

TABLE 2
Parameters for calculating Clinical Activity Score

| Item No. | Parameter |
|---|---|
| 1 | Spontaneous retrobulbar pain |
| 2 | Pain on attempted eye movements (upward, |
| | side to side, and downward gazes; sometimes |
| | termed gaze evoked orbital pain |
| 3 | Eyelid swelling |
| 4 | Eyelid erythema (redness) |
| 5 | Conjunctival redness |
| 6 | Chemosis (swelling/edema of the conjunctiva) |
| 7 | Swelling of caruncle or pila |

As provided in Table 2, the CAS consists of seven components: spontaneous retrobulbar pain, pain on attempted eye movements (upward, side-to-side, and downward gazes), conjunctival redness, redness of the eyelids, chemosis, swelling of the caruncle/plica, and swelling of the eyelids. Each component is scored as present (1 point) or absent (0 points). The score at each efficacy assessment is the sum of all items present; giving a range of 0-7, where 0 or 1 constitutes inactive disease and 7 severe active ophthalmopathy. A change of >2 points is considered clinically meaningful.

Item 1, spontaneous orbital pain could be a painful, or oppressive feeling on, or behind, the globe. This pain may be caused by the rise in intraorbital pressure, when the orbital tissues volume increases through excess synthesis of extracellular matrix, fluid accumulation, and cellular infiltration and expansion. Item 2, gaze evoked orbital pain, could be pain in the eyes when looking, or attempting to look, up, down or sideways, i.e., pain with upward, downward, or lateral eye movement, or when attempting eye movement. This kind of pain could arise from the stretching of the inflamed muscle(s), especially on attempted upgaze. The ‘stretching pain’ cannot be provoked by digital pressing on the eyeball, as would be expected if it were a manifestation of the raised intraorbital pressure. Both kinds of pain can be reduced after anti-inflammatory treatment. These kinds of pain are therefore considered to be directly related to autoimmune inflammation in the orbit and thus useful in assessing TAO activity.

Swelling in TAO is seen as chemosis (edema of the conjunctiva), item no. 6 in Table 1, and swelling of the caruncle and/or plica semilunaris. Both are signs of TAO activity. Swollen eyelids can be caused by edema, fat prolapse through the orbital septum, or fibrotic degeneration. In addition to swelling, other symptoms indicative of active TAO include redness and/or pain of the conjunctiva, eyelid, caruncle and/or plica semilunaris.

In some embodiments, the subject who is treated has the proptosis is reduced by at least 2 mm. In some embodiments, the subject who is treated has the proptosis is reduced by at least 3 mm. In some embodiments, the subject who is treated has the proptosis is reduced by at least 4 mm.

In some embodiments, in the subjects who are treated the clinical activity score (CAS) of the subject is reduced by at least 2 points. In some embodiments, the clinical activity score (CAS) of the subject is reduced to one (1). In some embodiments, the clinical activity score (CAS) of the subject is reduced to zero (0).

In some embodiments, methods off treating or reducing the severity of thyroid-associated ophthalmopathy (TAO) in a subject are provided, wherein the treatment with said antibody (i) reduces proptosis by at least 2 mm in an eye; (ii) is not accompanied by a deterioration of 2 mm or more in the other (or fellow eye); and (iii) reduces the CAS in said subject to either one (1) or zero (0).

In some embodiments, methods of improving the quality of life in a subject with thyroid-associated ophthalmopathy (TAO, also called Graves' Ophthalmopathy/Graves' Orbitopathy) are provided. In some embodiments, the quality of life is measured by the Graves' Ophthalmopathy Quality of Life (GO-QoL) assessment, or either the Visual Functioning or Appearance subscale thereof. In some embodiments, the treatment results in an improvement of greater than or equal to 8 points on the GO-QoL. In some embodiments, the treatment results in an improvement on the Functioning subscale of the GO-QoL. In some embodiments, the treatment results in an improvement on the Appearance subscale of the GO-QoL.

In some embodiments, methods of treating or reducing the severity of diplopia in a subject with thyroid-associated ophthalmopathy (TAO) are provided. In some embodiments, the diplopia is constant diplopia. In some embodiments, the diplopia is inconstant diplopia. In some embodiments, the diplopia is intermittent diplopia. In some embodiments, the improvement in or reduction in severity of diplopia is sustained at least 20 weeks after discontinuation of antibody administration. In some embodiments, the improvement in or reduction in severity of diplopia is sustained at least 50 weeks after discontinuation of antibody administration.

The severity of the disease can be measured in the following non-limiting embodiments. For example, for lid aperture, the distance between the lid margins are measured (in mm) with the patient looking in the primary position, sitting relaxed, and with distant fixation. For swelling of the eyelids, the measure/evaluation is either “absent/equivocal,” “moderate,” or “severe.” Redness of the eyelids is either absent or present. Redness of the conjunctivae is either absent or present. In some embodiments, conjunctival edema is either absent or present. In some embodiments, inflammation of the caruncle or plica is either absent or present. Exophthalmos is measured in millimeter using the same Hertel exophthalmometer and same intercanthal distance for an individual patient. Subjective diplopia is scored from 0 to 3 (0=no diplopia; 1=intermittent, i.e., diplopia in primary position of gaze, when tired or when first awakening; 2=inconstant, i.e., diplopia at extremes of gaze; 3=constant, i.e., continuous diplopia in primary or reading position). For eye muscle involvement, the ductions are measured in degrees. Corneal involvement is either absent/punctate or keratopathy/ulcer. For optic nerve involvement, i.e., best-corrected visual acuity, color vision, optic disc, relative afferent pupillary defect, the condition is either absent or present. In addition, visual fields are checked if optic nerve compression is suspected. In some embodiments, the patient can be classified according to the following severity classification. For example, sight-Threatening Thyroid Eye Disease: Patients with dysthyroid optic neuropathy (DON) and/or corneal breakdown. This category warrants immediate intervention. Moderate-to-Severe Thyroid Eye Disease: Patients without sight-threatening disease whose eye disease have sufficient impact on daily life to justify the risks of immunosuppression (if active) or surgical intervention (if inactive). Patients with moderate-to-severe thyroid eye disease usually have any one or more of the following: lid retraction greater than or equal to 2 mm, moderate or severe soft tissue involvement, exophthalmos greater than or equal to 3 mm above normal for race and gender, inconstant or constant diplopia. Mild Thyroid Eye Disease: Patients whose features of thyroid eye disease have only a minor impact on daily life insufficient to justify immunosuppressive or surgical treatment. They usually have only one or more of the following: minor lid retraction (<2 mm), mild soft tissue involvement, exophthalmos <3 mm above normal for race and gender, transient or no diplopia, and corneal exposure responsive to lubricants.

In some embodiments, a patient can be characterized by Graves Ophthalmopathy Quality of Life (GO-QoL) score. In addition to proptosis (or exophthalmos) and CAS, quality of life is also evaluated with the use of the GO quality of life (GO-QoL) questionnaire. This questionnaire is designed to determine the improved quality of life after treatment with a method disclosed herein. In some embodiments, questionnaire may determine the decreased or lack of side effects after being treated with an antibody, or an antigen binding fragment thereof, according to a method disclosed herein as compared to treatment with glucocorticoids. The GO-QoL is a 16-item self-administered questionnaire divided into 2 subsets and used to assess the perceived effects of TED by the subjects on (i) their daily physical activity as it relates to visual function, and (ii) psychosocial functioning. Quality of life is evaluated with the use of the GO QoL questionnaire. The GO-QoL questionnaire [C. B. Terwee et al, 1998] is completed on Day 1 and Weeks 6, 12, and 24 (or PW) during the Treatment Period, and at Months 7 and 12 (or PW) during the Follow-Up Period. The GO-QoL is a 16-item self-administered questionnaire divided into two self-assessment subscales; one covering impact of visual function on daily activities, the other assesses the impact of self-perceived appearance. The visual function subscale covers activities such as driving, walking outdoors, reading, watching television. The appearance subscale asks the subject questions such as whether ophthalmopathy has altered the subject's appearance, caused other people to have a negative reaction to the subject, caused social isolation, and caused the subject to try to mask his or her appearance. Each subscale has 8 questions which are answered with: yes—very much so; yes—a little; or no—not at all. Each question is scored 0-2, respectively, and the total raw score is then mathematically transformed to a 0-100 scale, where 0 represents the most negative impact on quality of life, and 100 represents no impact. A change of >or greater than equal to 8 points on the 0-100 scale has been shown to be clinically meaningful. The combined score takes raw scores from both subscales and again transforms them to a single 0-100 scale. The questionnaire has two self-assessment subscales. Each subscale has 8 questions which are answered with: (i) yes—very much so; (ii) yes—a little; or (iii) no—not at all. Each question is scored 0-2, respectively, and the total raw score is then mathematically transformed to a 0-100 scale, where 0 represents the most negative impact on quality of life, and 100 represents no impact. A change of >8 points on the 0-100 scale is considered to be clinically meaningful. The combined score takes raw scores from both subscales and again transforms them to a single 0-100 scale.

Patients can also be assessed by the presence of absence of Gorman Grading of Diplopia. The Gorman assessment of subjective diplopia includes four categories: no diplopia (absent), diplopia when the patient is tired or awakening (intermittent), diplopia at extremes of gaze (inconstant), and continuous diplopia in the primary or reading position (constant). Patients are scored according to which grade of diplopia they are experiencing. An improvement of greater than equal or to 1 grade is considered clinically meaningful.

In some embodiments, the methods comprise administering an antibody, such as those provided herein. In some embodiments, the antibody is administered at a dosage of about 1 mg/kg to about 5 mg/kg antibody as a first dose. In some embodiments, the antibody is administered at a dosage of about 5 mg/kg to about 10 mg/kg antibody as a first dose. In some embodiments, the antibody is administered at a dosage of about 5 mg/kg to about 20 mg/kg antibody in subsequent doses. In some embodiments, the antibody is administered in the following amounts: about 10 mg/kg antibody as a first dose; and about 20 mg/kg antibody in subsequent doses. In some embodiments, the subsequent doses are administered every three weeks for at least 21 weeks.

In some embodiments, the antibody is administered in a pharmaceutical composition, such as those provided herein. In some embodiments, the pharmaceutical composition further comprises one or more pharmaceutically active compounds for the treatment of TAO. In some embodiments, the pharmaceutical composition further comprises corticosteroids; rituximab or other anti-CD20 antibodies; tocilizumab or other anti-IL-6 antibodies; or selenium, infliximab or other anti-TNFalpha antibodies or a thyroid-stimulating hormone receptor (TSHR) inhibitor.

In some embodiments, the method provided herein comprise administering to a subject an antibody, or an antigen binding fragment thereof, that specifically binds to and inhibits IGF-IR. In some embodiments, the antibody is as provided herein.

Kits are also provided which are useful for carrying out embodiments described herein. The present kits comprise a first container containing or packaged in association with the above-described antibodies. The kit may also comprise another container containing or packaged in association solutions necessary or convenient for carrying out the embodiments. The containers can be made of glass, plastic or foil and can be a vial, bottle, pouch, tube, bag, etc. The kit may also contain written information, such as procedures for carrying out the embodiments or analytical information, such as the amount of reagent contained in the first container means. The container may be in another container apparatus, e.g. a box or a bag, along with the written information.

Yet another aspect provided for herein is a kit for detecting IGF-1R protein in a biological sample. The kit includes a container holding one or more antibodies which binds an epitope of IGF-1R protein and instructions for using the antibody for the purpose of binding to IGF-1R protein to form an immunological complex and detecting the formation of the immunological complex such that the presence or absence of the immunological complex correlates with presence or absence of IGF-1R protein in the sample. Examples of containers include multiwell plates which allow simultaneous detection of IGF-1R protein in multiple samples.

In some embodiments, antibodies that bind to a IGF-1R protein are provided. In some embodiments, the antibody is isolated. In some embodiments, the antibody binds specifically. In some embodiments, the antibody binds to a IGF-1R protein that is properly folded. In some embodiments, the antibody is specific for a specific IGF-1R conformational state (open or closed). In some embodiments, the antibody binds to a IGF-1R protein in a cell membrane. In some embodiments, the antibody binds to a IGF-1R protein that is in a cell membrane in an intact cell. In some embodiments, the antibody inhibits or neutralizes the function of a IGF-1R protein. As used herein, the term “neutralize” means that the activity or function of the protein is inhibited. The inhibition can be complete or partial. In some embodiments, the activity or function of the protein is inhibited at least 10, 20, 30, 40, 50, 60, 70, 80, 90, 95, or 99%. The percent inhibition can be based upon the function or activity of the protein in the absence of the antibody. In some embodiments, the antibody inhibits the glucose transport facilitated by IGF-1R. In some embodiments, the antibody inhibits the internalization of the IGF-1R protein.

In some embodiments, the antibody comprises a sequence as provided for herein or antigen binding fragment thereof. In some embodiments, the antibody comprises a heavy chain CDR or an antigen binding fragment thereof described herein. The heavy chain may be one or more of the heavy chains described herein. In some embodiments, the antibody comprises a light chain, or an antigen binding fragment thereof as described herein

In some embodiments, methods of treating, inhibiting or ameliorating a IGF-1R, associated pathology are provided. In some embodiments, the methods comprise administering an antibody described herein or a pharmaceutical composition described herein to a subject to treat, inhibit or ameliorate a IGF-1R associated pathology. In some embodiments, the pathology is as described herein.

In some embodiments, methods of detecting the presence or absence of a IGF-1R in a sample are provided, the method comprising contacting a sample with one or more antibodies described herein detecting the binding to a IGF-1R antigen by the antibody. In some embodiments, the detection of the binding indicates the presence IGF-1R antigen; or the absence of the detection of the binding to the IGF-1R antigen indicates the absence of the IGF-1R antigen. The detecting can be done with any known method, such as using a biosensor, ELISA, sandwich assay, and the like. However, in some embodiments, the method comprises detecting the presence of the protein in non-denaturing conditions. The non-denaturing conditions can be used so that the protein of interest is detected in its native, or properly folded form.

In some embodiments, methods of identifying a test antibody that binds to an epitope on IGF-1R protein, are provided, the method comprising contacting a test antibody with the epitope on IGF-1R protein and determining whether the test antibody binds to the epitope. In some embodiments, the determining comprises determining whether the test antibody binds to the protein and is competitively inhibited by an antibody comprising a sequence as provided herein. In some embodiments, the determining comprises mutating one or more residues of epitope or protein and determining binding of the test antibody to the mutated epitope, wherein if the mutation reduces binding of the test antibody as compared to the non-mutated epitope, the test antibody is deemed to bind to that epitope.

In some embodiments, methods of monitoring internalization of IGF-1R from the surface of a cell are provided. In some embodiments, the method comprising contacting the cell with an anti-IGF-1R antibody as provided herein and detecting the presence of IGF-1R in the cell or on the surface of the cell. The differences in cell surface expression can be measured and the internalization can be monitored and measured. This can be used, for example, to measure the effect of another molecule, such as a test agent, to modulate internalization of IGF-1R protein. Thus, the antibodies provided for herein can be used to identify test agents that modulate (increase or decrease) the internalization of IGF-1R protein. Test molecules that increase the internalization, which would be measured as a decrease in binding of an anti-IGF-1R antibody to IGF-1R protein on the cell surface, can be identified according to the methods provided herein. Test molecules that decrease the internalization, which would be measured as an increase in binding of an anti-IGF-1R antibody to IGF-1R protein on the cell surface, can be identified according to the methods provided herein. The surface expression can be measured by fluorescence, which can be done through a secondary antibody that recognized the IGF-1R antibodies or by labelling the anti-IGF-1R antibodies provided for herein.

In some embodiments, methods of inhibiting IGF-1 stimulated receptor phosphorylation on a cell are provided. In some embodiments, the methods comprise contacting the cell with an antibody as provided for herein, or a pharmaceutical composition comprising the same. In some embodiments, the contacting comprises administering to a subject the antibody or a pharmaceutical composition comprising the same. In some embodiments, the cell is a cell in the eye. In some embodiments, the subject has or is at risk of thyroid eye disease (TED). In some embodiments, the antibody has an IC50 of less than, or equal to, about 0.2 nm, 0.15 nm, 0.10 nm, 0.09 nm. In some embodiments, the IC50 is measured in an in vitro assay, such as an assay as provided for herein, such as illustrated in the Examples. In some embodiments, the IC50 is measured in an cell that is an A549 cell or a HOCF cell.

In some embodiments, methods of treating thyroid eye disease in a subject are provided, the method comprising administering an antibody as provided for herein, or a pharmaceutical composition comprising the same to the subject, wherein the antibody has a serum concentration in the subject of at least, or about, 70 μg/ml, 75 μg/ml, 80 μg/ml, 85 μg/ml, 90 μg/ml, 95 μg/ml, 100 μg/ml, or 105 μg/ml at least 1, 2, or 3 weeks after administration. In some embodiments, the serum concentration is measured after one, two or three doses of the antibody, or the pharmaceutical composition comprising the same, are administered to the subject.

In some embodiments, methods of inhibiting IGF-1 induced receptor autophosphorylation by at least 95%, 96%, 97%, 98%, or 99% or by 100% in a subject in need thereof are provided. In some embodiments, the methods comprise administering to the subject an antibody as provided for herein, or a pharmaceutical composition comprising the same. In some embodiments, the IGF-1 induced receptor autophosphorylation is inhibited in the eye or orbital region of the subject. In some embodiments, the IGF-1 induced receptor autophosphorylation is inhibited thereby treating a subject for thyroid eye disease or improving a symptom as described herein.

### Enumerated Embodiments

In some embodiments, embodiments provided herein also include, but are not limited to:

1. An antibody, or antigen binding fragment thereof, comprising:
a heavy chain complementarity-determining region (HCDR) sequence selected from the group consisting of SEQ ID NO: 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, and 321; and
a light chain CDR (LCDR) sequence selected from the group consisting of SEQ ID NO: 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, a353, 354, 355, 356, 357, 358, 359, 360, 361, and 362.
   2. An antibody, or antigen binding fragment thereof, comprising:
a heavy chain variable region (VH) sequence selected from the group consisting of SEQ ID NO: 1, 3, 4, 5, 6, 7, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 67, 68, 69, 70, 71, 72, 73, 74, 75, 76, 77, 78, 79, 80, 81, 82, 83, 84, 85, 86, 87, 88, 99, 101, 102, 103, 104, 105, 106, 107, 108, 110, 111, 112, 113, 114, 115, 116, 117, 118, 119, 120, and 121; and
a light chain variable region (VL) sequence selected from the group consisting of SEQ ID NO: 2, 8, 39, 40, 41, 42, 43, 44, 45, 46, 47, 48, 49, 50, 51, 52, 53, 54, 55, 56, 57, 58, 59, 60, 61, 62, 63, 64, 65, 66, 89, 90, 91, 92, 93, 94, 95, 96, 97, 98, 100, 109, and 122.
   3. The antibody of any one of embodiments 1 or 2, or antigen binding fragment thereof, wherein the antibody binds to insulin-like growth factor I receptor (IGF-1R).
   4. The antibody of any one of embodiments 1-3, wherein the antibody is a monoclonal antibody.
   5. The antibody of any one of embodiments 1-4, wherein the antibody is a humanized antibody.
   6. The antibody of any one of embodiments 1-5, wherein the antibody is a scFv antibody.
   7. The antibody of any one of embodiments 1-6, wherein the antibody, or antigen binding fragment thereof, comprises a VH peptide comprising an amino acid sequence as set forth in SEQ ID NOs: 1, 3, 4, 5, 6, 7, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 67, 68, 69, 70, 71, 72, 73, 74, 75, 76, 77, 78, 79, 80, 81, 82, 83, 84, 85, 86, 87, 88, 99, 101, 102, 103, 104, 105, 106, 107, 108, 110, 111, 112, 113, 114, 115, 116, 117, 118, 119, 120, or 121, or any variant thereof.
   8. The antibody of any one of embodiments 1-7, wherein the antibody, or antigen binding fragment thereof, comprises a VL peptide comprising an amino acid sequence as set forth in SEQ ID NOs: 2, 8, 39, 40, 41, 42, 43, 44, 45, 46, 47, 48, 49, 50, 51, 52, 53, 54, 55, 56, 57, 58, 59, 60, 61, 62, 63, 64, 65, 66, 89, 90, 91, 92, 93, 94, 95, 96, 97, 98, 100, 109, or 122, or any variant thereof.
   9. An antibody, or antigen binding fragment thereof, wherein the antibody or antibody fragment comprises: (i) a heavy chain variable region comprising HCDR1, HCDR2, and HCDR3 sequences, wherein the HCDR1 sequence has the amino acid sequence of SEQ ID NO: 263, 264, 265, 266, 267, 268, 269, 270, 271, 272, 273, 274, 275, or 276; the HCDR2 has the amino acid sequence of SEQ ID NO: 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, or 397; and the HCDR3 sequence has the amino acid sequence of SEQ ID NO: 311, 312, 313, 314, 315, 316, 317, 318, 319, 320, or 321; or variants of any of the foregoing; and (ii) a light chain variable region comprising LCDR1, LCDR2, and LCDR3 sequences, wherein the LCDR1 sequence has the amino acid sequence SEQ ID NO: 322, 323, 324, 325, 326, 327, 328, 329, 330, 331, 332, 333, 334, 335, 336, 337, or 338; the LCDR2 sequence has the amino acid sequence of SEQ ID NO: 339, 340, 341, 342, 343, 344, 345, 346, 347, 348, 349, or 350; and the LCDR3 sequence has the amino acid sequence of SEQ ID NO: 351, 352, 353, 354, 355, 356, 357, 358, 359, 360, 361, or 362; or variants of any of the foregoing.
   10. An antibody, or antigen binding fragment thereof, comprising a VH and VL pair comprising the amino acid sequence of SEQ ID NOs:123-262, or a variant thereof.
   11. The antibody of any one of embodiments 1-10, wherein the heavy chain variable region and the light chain variable region are not linked by a linker.
   12. The antibody of any one of embodiments 1-10, wherein the heavy chain variable region and the light chain variable region are linked with a peptide linker.
   13. The antibody of embodiment 12, wherein the peptide linker comprises a sequence of: (GGGGS)ₙ (SEQ ID NO: 375) (GGGGA)ₙ (SEQ ID NO: 376), or any combination thereof, wherein each n is independently 1-5.
   14. The antibody of any one of embodiments 1-13, wherein the variant has 1-10 substitutions, deletions, or insertions.
   15. The antibody of any one of embodiments 1-14, wherein the variant has 1-10 conservative substitutions.
   16. The antibody of any one of embodiments 1-15, wherein the variant has at least 85%, 90%, 91%, 92%, 93%, 94%, 95%, 96%, 97%, 98%, or 99% homology to a sequence of SEQ ID NOs: 1-122.
   17. The antibody of any one of embodiments 1-15, wherein the variant has at least 85%, 90%, 91%, 92%, 93%, 94%, 95%, 96%, 97%, 98%, or 99% homology to a sequence of SEQ ID NOs: 123-262.
   18. The antibody of any one of embodiments 1-15, wherein the variant has at least 85%, 90%, 91%, 92%, 93%, 94%, 95%, 96%, 97%, 98%, or 99% identify to a sequence of SEQ ID NO: 263-362, and 397.
   19. The antibody of any one of embodiments 1-18, wherein the antibody is a scFv antibody.
   20. The antibody of any one of embodiments 1-19, wherein the antibody is a monoclonal antibody.
   21. The antibody of any one of embodiments 1-20, wherein the antibody is a humanized antibody.
   22. The antibody of any one of embodiments 1-21, wherein the antibody comprises a Fc region.
   23. The antibody of embodiment 22, wherein the Fc region is selected from the group consisting of SEQ ID NO: 363, 364, 365, 366, 367, 368, 369, and 370.
   24. The antibody of any one of embodiments 1-23, wherein the Fc region comprises a mutation that extends the half-life of the antibody when linked to the Fc region.
   25. The antibody of embodiment 24, wherein the Fc region comprises a S228P, L235E, M252Y, S254T, T256E, M428L, N434S, L234F, P331S mutation, or any combination thereof.
   26. The antibody of embodiment 24, wherein the Fc region comprises a M252Y, S254T, and T256E mutation.
   27. The antibody of embodiment 24, wherein the Fc region comprises a S228P and a L235E mutation.
   28. The antibody of embodiment 24, wherein the Fc region comprises a L234F, L235E, and P331S mutation.
   29. The antibody of embodiment 24, wherein the Fc region comprises M252Y, S254T, T256E, S228P and L235E mutations.
   30. The antibody of embodiment 24, wherein the Fc region comprises S228P, L235E, M428L, and N434S mutations.
   31. The antibody of embodiment 24, wherein the Fc region comprises M428L and N434S mutations.
   32. The antibody of embodiment 24, wherein the Fc region comprises L234F, L235E, P331S, M252Y, S254T, and T256E mutations.
   33. The antibody of any one of embodiments 1-32, wherein the antibody is an isolated antibody.
   34. A nucleic acid molecule encoding an antibody, or antigen binding fragment thereof, of any one of embodiments 1-33.
   35. A vector comprising the nucleic acid molecule of embodiment 34.
   36. A cell comprising the nucleic comprising the nucleic acid molecule of embodiment 34 or the vector of embodiment 35.
   37. A pharmaceutical composition comprising the antibody of any one of embodiments 1-33 or a nucleic acid molecule encoding the same.
   38. The pharmaceutical composition of embodiment 37, wherein the composition is an injectable pharmaceutical composition.
   39. The pharmaceutical composition of embodiments 37 or 38, wherein the pharmaceutically composition is administered intravenously or subcutaneously.
   40. A method of treating or reducing the severity of, thyroid-associated ophthalmopathy (TAO), or a symptom thereof, comprising administering to a subject an antibody of any one of embodiments 1-33 or a pharmaceutical composition of any one of embodiments 37-39.
   41. A method of reducing proptosis in an eye in a subject with thyroid-associated ophthalmopathy (TAO) comprising administering to a subject an antibody of any one of embodiments 1-33 or a pharmaceutical composition of any one of embodiments 37-39.
   42. A method of treating thyroid eye disease in a subject comprising administering to a subject an antibody of any one of embodiments 1-33 or a pharmaceutical composition of any one of embodiments 37-39.
   43. A method of reducing Clinical Activity Score (CAS) of thyroid-associated ophthalmopathy (TAO) in a subject comprising administering to a subject an antibody of any one of embodiments 1-33 or a pharmaceutical composition of any one of embodiments 37-39.
   44. A method of a) reducing proptosis by at least 2 mm and b) reducing the clinical activity score (CAS) in a subject with thyroid-associated ophthalmopathy (TAO) comprising administering to a subject an antibody of any one of embodiments 1-33 or a pharmaceutical composition of any one of embodiments 37-39.
   45. The method of any of embodiments 40-44, wherein proptosis is reduced by at least 2 mm.
   46. The method of any of embodiments 40-44, wherein proptosis is reduced by at least 3 mm.
   47. The method of any of embodiments 40-44, wherein proptosis is reduced by at least 4 mm.
   48. The method of any of embodiments 40-44, wherein the clinical activity score (CAS) of the subject is reduced by at least 2 points.
   49. The method of any of embodiments 40-44, wherein the clinical activity score (CAS) of the subject is reduced to one (1).
   50. The method of any of embodiments 40-44, wherein the clinical activity score (CAS) of the subject is reduced to zero (0).
   51. A method of treating or reducing the severity of thyroid-associated ophthalmopathy (TAO) in a subject comprising administering to a subject an antibody of any one of embodiments 1-33 or a pharmaceutical composition of any one of embodiments 36-38, wherein treatment with said antibody (i) reduces proptosis by at least 2 mm in an eye; (ii) is not accompanied by a deterioration of 2 mm or more in the other (or fellow eye); and (iii) reduces the CAS in said subject to either one (1) or zero (0).
   52. A method of improving the quality of life in a subject with thyroid-associated ophthalmopathy (TAO, also called Graves' Ophthalmopathy/Graves' Orbitopathy) comprising administering to a subject an antibody of any one of embodiments 1-33 or a pharmaceutical composition of any one of embodiments 37-39.
   53. The method of embodiment 52, wherein the quality of life is measured by the Graves' Ophthalmopathy Quality of Life (GO-QoL) assessment, or either the Visual Functioning or Appearance subscale thereof.
   54. The method of embodiment 53, wherein the treatment results in an improvement of greater than or equal to 8 points on the GO-QoL.
   55. The method of embodiment 53, wherein the treatment results in an improvement on the Functioning subscale of the GO-QoL.
   56. The method of embodiment 53, wherein the treatment results in an improvement on the Appearance subscale of the GO-QoL.
   57. A method of treating or reducing the severity of diplopia in a subject with thyroid-associated ophthalmopathy (TAO) comprising administering to a subject an antibody of any one of embodiments 1-33 or a pharmaceutical composition of any one of embodiments 37-39.
   58. The method of embodiment 57, wherein the diplopia is constant diplopia.
   59. The method of embodiment 57, wherein the diplopia is inconstant diplopia.
   60. The method of embodiment 57, wherein the diplopia is intermittent diplopia.
   61. The method of embodiment 57, wherein the improvement in or reduction in severity of diplopia is sustained at least 20 weeks after discontinuation of antibody administration.
   62. The method of embodiment 57, wherein the improvement in or reduction in severity of diplopia is sustained at least 50 weeks after discontinuation of antibody administration.
   63. The method of any one of embodiments 40-62, wherein said antibody is administered at a dosage of about 1 mg/kg to about 5 mg/kg antibody as a first dose.
   64. The method of any one of embodiments 40-62, wherein said antibody is administered at a dosage of about 5 mg/kg to about 10 mg/kg antibody as a first dose.
   65. The method of any one of embodiments 40-62, wherein said antibody is administered at a dosage of about 5 mg/kg to about 20 mg/kg antibody in subsequent doses.
   66. The method of any one of embodiments 40-62, wherein said antibody is administered in the following amounts: about 10 mg/kg antibody as a first dose; and about 20 mg/kg antibody in subsequent doses.
   67. The method of embodiment 66, wherein said subsequent doses are administered every three weeks for at least 21 weeks.
   68. The method of any one of embodiments 40-67, wherein the antibody, or an antigen binding fragment thereof, is a human antibody, a monoclonal antibody, a human monoclonal antibody, a purified antibody, a diabody, a single-chain antibody, a multi-specific antibody, Fab, Fab′, F(ab′)2, Fv or scFv.
   69. The method of any one of embodiments 40-68, wherein the antibody, or an antigen binding fragment thereof, is administered in a pharmaceutical composition that additionally comprises a pharmaceutically acceptable diluent or excipient or carrier.
   70. The method of embodiment 69, wherein the pharmaceutical composition further comprises one or more pharmaceutically active compounds for the treatment of TAO.
   71. The method of embodiment 69 or 70, wherein the pharmaceutical composition further comprises corticosteroids; rituximab or other anti-CD20 antibodies; tocilizumab or other anti-IL-6 antibodies; or selenium, infliximab or other anti-TNFalpha antibodies or a thyroid-stimulating hormone receptor (TSHR) inhibitor.
   72. The method of any one of the embodiments 4-71, wherein the antibody or an antigen binding fragment thereof is administered directly to the eye, the anterior chamber of the eye, the vitreous chamber of the eye, the suprachoroidal space, or the retro-orbital sinus.
   73. The method of embodiment 72, wherein the antibody or an antigen binding fragment thereof is administered via an injection.
   74. The method of embodiment 73, wherein the injection is a intravitreal injection, intraorbital injection, retro-orbital injection, suprachoroidal injection, or intracameral injection.
   75. A method of increasing the internalization of IGF-1R on a cell, the method comprising contacting the cell with an antibody of any one of embodiments 1-33 or a pharmaceutical composition of any one of embodiments 37-39.
   76. The method of embodiment 74, wherein the contacting comprises administering to a subject an antibody of any one of embodiments 1-33 or a pharmaceutical composition of any one of embodiments 37-39.
   77. The method of embodiment 76, wherein the subject has or is at risk of thyroid eye disease (TED).
   78. A method of inhibiting IGF-1 stimulated receptor phosphorylation on a cell, the method comprising contacting the cell with an antibody of any one of embodiments 1-33 or a pharmaceutical composition of any one of embodiments 37-39.
   79. The method of embodiment 78, wherein the contacting comprises administering to a subject an antibody of any one of embodiments 1-33 or a pharmaceutical composition comprising of any one of embodiments 36-38.
   80. The method of embodiment 79, wherein the subject has or is at risk of thyroid eye disease (TED).
   81. The method of any one of embodiments 78-80, wherein the antibody has an IC50 of less than, or equal to, about 0.2 nm, 0.15 nm, 0.10 nm, 0.09 nm.
   82. The method of embodiment 81, wherein the IC50 is measured in an in vitro assay, such as an assay as provided for herein.
   83. The method of any one of embodiments 78-82, wherein the cell is an A549 cell or a HOCF cell.
   84. A method of treating thyroid eye disease in a subject, the method comprising administering an antibody of any one of embodiments 1-33, or a pharmaceutical composition of any one of embodiments 37-39 to the subject, wherein the antibody has a serum concentration in the subject of at least, or about, 70 μg/ml, 75 μg/ml, 80 μg/ml, 85 μg/ml, 90 μg/ml, 95 μg/ml, 100 μg/ml, or 105 μg/ml at least 1, 2, or 3 week after administration.
   85. The method of embodiment 84, wherein the antibody or the pharmaceutical composition is administered intravenously.
   86. The method of any one of embodiments 84-85, wherein the antibody or the pharmaceutical composition is administered at a dose of about 20 mg/kg.
   87. The method of any one of embodiments 84-86, wherein the antibody or the pharmaceutical composition is administered at least, or about, once a week, once every two weeks, once every 3 weeks, or once every 4 weeks.
   88. A method of inhibiting IGF-1 induced receptor autophosphorylation in a cell by at least 95%, 96%, 97%, 98%, or 99% or by 100%, the method comprising contacting the cell with an antibody of any one of embodiments 1-33, or a pharmaceutical composition of any one of embodiments 37-39.
   89. The method of embodiment 88, wherein the inhibition of the IGF-1 induced receptor autophosphorylation is measured as compared to the induced receptor autophosphorylation in the absence of the antibody or the pharmaceutical composition.
   90. The method of embodiments 88 or 89, wherein the contacting comprises administering to a subject the antibody or the pharmaceutical composition comprising the same.
   91. The method of embodiment 90, wherein the subject has or is at risk of thyroid eye disease (TED).
   92. A method of inhibiting IGF-1 induced receptor autophosphorylation by at least 95%, 96%, 97%, 98%, or 99% or by 100% in a subject in need thereof, the method comprising administering to the subject an antibody of any one of embodiments 1-33, or a pharmaceutical composition of any one of embodiments 37-39. 93. The method of embodiment 92, wherein the subject has or is at risk of thyroid eye disease (TED).
   94. The method of any one of embodiments 92 or 93, wherein the antibody or the pharmaceutical composition is administered intravenously.
   95. The method of any one of embodiments 88-94, wherein the antibody comprises the CDRs of VRDN-1100. 96. The method of any one of embodiments 88-95, wherein the antibody comprises the CDRs of the antibody of VRDN-1100 or the CDRs of VRDN-2700.

### EXAMPLES

The subject matter is now described with reference to the following examples. These examples are provided for the purpose of illustration only and the claims should in no way be construed as being limited to these examples, but rather should be construed to encompass any and all variations which become evident as a result of the teaching provided herein. Those of skill in the art will readily recognize a variety of non-critical parameters that could be changed or modified to yield essentially similar results.

### Example 1: IGF-1R Antibodies Block IGF-1 Stimulation

Blockage of IGF-1 stimulation is measured by secretion of hyaluronan, in the presence of IGF-1R antibodies VRDN-2700, VRDN-03100, VRDN-02100, VRDN-02200, VRDN-02300, VRDN-02400, and VRDN-02500, all of which are disclosed herein. Immunoglobulins are purified from the sera of patients with Graves' ophthalmopathy (GO) and tested for their ability to activate TSHR and/or IGF-1R directly, and TSHR/IGF-1R cross talk in primary cultures of GO fibroblasts. Cells are treated with M22 or GO-Igs with or without IGF-1R inhibitory antibodies such as those provided for herein, including but not limited to, VRDN-2700, VRDN-03100, VRDN-02100, VRDN-02200, VRDN-02300, VRDN-02400, and VRDN-02500, all of which are disclosed herein. Hyaluronan (hyaluronic acid; HA) secretion is measured as a major biological response for GO fibroblast stimulation. IGF-1R autophosphorylation is used as a measure of direct IGF-1R activation. TSHR activation is determined through cyclic-AMP (cAMP) production. The IGF-1R antibodies, as disclosed herein, are found to effectively block HA secretion and, therefore, are found to block IGF stimulation.

### Example 2: Treatment of Patients with Thyroid Eye Disease and Clinical Assessment of IGF-1R Antibodies on Thyroid Eye Disease

Infusions of IGF-1R inhibitory antibodies such as those provided for herein, including but not limited to, VRDN-2700, VRDN-03100, VRDN-02100, VRDN-02200, VRDN-02300, VRDN-02400, and VRDN-02500, all of which are disclosed herein, are provided to the subjects. The number of infusions is individualized for each subject and is based on the investigator's clinical judgment. The Day 1 Visit occurs within 14 days after the final visit of the prior trial. Visit windows are ±1 day for Weeks 1 and 4, ±3 days for Weeks 3, 6, 9, 12, 15, 18, 21, and 24. The Follow-up period is meant for subjects who were proptosis non-responders in the prior trial only; subjects who relapsed in the prior trial did not participate in the Follow-Up Period. Visit windows during the Follow-up period are ±7 days.

Treatment Period is 24 weeks (6 months), during which 8 infusions of teprotumumab are administered.

Subjects who are proptosis non-responders are scheduled to participate in a 6-month Follow-Up Period in this extension study; subjects who relapsed in the lead-in study and are retreated in this extension study will not participate in the Follow-Up Period.

Efficacy assessments are performed for both eyes at each assessment time point. The “study eye” (i.e., the more severely affected eye) will remain the same as that identified at the Baseline (Day 1) Visit of the prior study. Both eyes are assessed for efficacy but the study eye is used to assess the primary outcome measure.

Efficacy is assessed by proptosis (measured as exophthalmos evaluation of the Clinical Measures of Severity using a Hertel instrument for consistency in measurement), CAS (7-item scale), diplopia (measured as part of the Clinical Measures of Severity) and Clinical Measures of Severity (including motility restriction assessments).

Quality of life is assessed using the GO-QoL questionnaire.

Safety is assessed via AE and concomitant medication use monitoring, immunogenicity testing, physical and ophthalmic examinations, vital signs, clinical safety laboratory evaluations (complete blood count, chemistry (including thyroid panel and HbA1C), and urinalysis), pregnancy testing (if applicable), and electrocardiograms (ECG). The study is also monitored by a Data Safety Monitoring Board (DSMB).

Proptosis assessments is performed using a Hertel exophthalmometer for consistency in measurement, and (except when strictly unavoidable) the same Hertel instrument and same observer is used at each evaluation for the full duration of the study. Additionally, the same intercanthal distance (ICD) is used on each occasion.

Proptosis is measured for each eye on Day 1 and Weeks 6, 12, 18, and 24 (or premature withdrawal (PW)) during the Treatment Period, and at Months 7, 9, and 12 (or PW) during the Follow-Up Period. Measurements is recorded on the Clinical Measures of Severity eCRF under exophthalmos.

The antibodies are found to be effective in treating thyroid eye disease and also improving quality of life as provided for herein.

### Example 3: Antibody with Increased pK

Cynomolgus monkeys were dosed with an antibody comprising the CDRs of VRDN-2700 with the YTE mutation in the Fc domain in an amount of 10 mg/kg by either intravenous or subcutaneous route, and samples were collected at 0.5 hr, 2 hr, 8 hr and days 1, 3, 7, 10, 14, 21, and 28 time points for PK analysis by ELISA. Teprotumumab was also administered at 10 mg/kg IV as a comparator. The results demonstrate that the antibody had a significantly higher PK as compared to Teprotumumab. This result demonstrates an antibody comprising the CDRs of VRDN-2700 can likely be given at a lower dose as compared to Teprotumumab, even when administered subcutaneously. These results could not have been predicted.

### Example 4: VRDN-2700

VRDN-2700, which has a M252Y, S254T, and T256E mutation in the Fc domain is a novel anti-IGF-1R antibody incorporating half-life extension modifications in its Fc region as described herein and can be used for the treatment of Thyroid Eye Disease (TED). The pharmacokinetic (PK) parameters of VRDN-2700 with such Fc mutations was measured in cynomolgus monkeys to the marketed IGF-1R antibody, teprotumumab, and a PK model was constructed to project potential human dosing regimens.

TED is an autoimmune condition most commonly associated with Graves' disease and hyperthyroidism but can also be found in patients who are euthyroid or hypothyroid. Orbitopathy in TED is driven by Thyroid Stimulating Hormone Receptor (TSHR) agonistic autoantibodies and crosstalk between TSHR and IGF-1R. Pathological remodeling of the orbit and periorbital tissues results in varied presentations which may include dry eyes, increased lacrimation, local irritation, eyelid retraction and eventually proptosis, diplopia, and optic nerve compression, with ensuing vision loss.

The underlying pathology of TED is the activation of an inflammatory cascade within the orbit, primarily due to recruitment of fibrocytes and immune cells. Over-expression of IGF-1R has been demonstrated within the orbit of TED patients, and it has been surmised that IGF-1R inhibitory antibodies may disrupt the IGF-1R and TSHR cross-talk and dampen the inflammatory cascade. Indeed, IGF-1R antagonism has been demonstrated to robustly relieve much of the inflammatory symptomology that affects TED patients.

VRDN-2700 is a monoclonal antibody that inhibits IGF-1 mediated signaling via IGF-1R with subnanomolar potency and incorporates clinically validated Fc modifications (M252Y, S254T, and T256E) to extend half-life. This antibody was found to have a more favorable PK profile with the potential for a less burdensome treatment paradigm for patients than conventional IgG therapeutic antibodies.

VRDN-2700 with the Fc mutations was administered to cynomolgus monkeys by 30 min intravenous (IV) infusions at 2, 10, and 50 mg/kg, and by subcutaneous (SC) injection at 2 and 10 mg/kg. Teprotumumab at 10 mg/kg was likewise administered by 30 min IV infusion. VRDN-2700 and teprotumumab levels in serum were measured using a human IgG specific ELISA assay. Data were analyzed using the WinNonlin non-compartmental model. A semi-mechanistic model incorporating target mediated drug disposition was constructed using available human and cynomolgus data. The data is illustrated below.

The data indicates the more favorable PK profile.

Evidence of target mediated drug disposition (TMDD) was observed at 2 mg/kg, but not at 10 and 50 mg/kg doses, in line with teprotumumab and other IGF-1R antibodies that have reported saturation of TMDD at higher doses.

VRDN-2700 Half-Life Extension Modifications Prolong Exposure. At equivalent doses, SC dosed VRDN-2700 with the YTE mutations has greater exposure than intravenously infused teprotumumab and achieves ˜2× half-life of teprotumumab in NHPs Estimated 62% bioavailability (F) of VRDN-2700 from SC dosing using preliminary discovery-stage formulation.

Model simulations predict that dosing of VRDN-2700 at 10 mg/kg every 3 weeks or 20 mg/kg every 6 weeks will result in Cmin of >100 μg/mL, similar to the approved teprotumumab regimen (10 mg/kg first dose followed by seven 20 mg/kg doses q3w). The 10 mg/kg q3w regimen will with lower Cmax values. A longer dosing interval would increase patient convenience and reduce treatment costs, while lower dose and Cmax values may potentially mitigate toxicities. Furthermore, the model predicts that weekly subcutaneous dosing of VRDN-2700 at 300 mg fixed dose could achieve a steady-state Cmin of ˜130 ug/mL, enabling at home self-administration. In the event that lower Cmin values are efficacious, subcutaneous administration of VRDN-2700 at 300 mg fixed dose every other week is predicted to achieve ˜50 ug/mL steady-state Cmin levels. Taken together, the extended half-life of VRDN-2700 is predicted to provide patients with a wider range of options for more convenient dosing interval and route of administration.

During the evaluation of the antibodies, expression of VRDN-2700 was compared to other antibodies having mutations in the Fc domain, such as the L/S mutations that are described herein. Unexpectedly, the yield for the antibody with the YTE mutation in the Fc domain (VRDN2700) was approximately 80% higher than the yield of a similar antibody except that it has a L/S mutation. This was surprising and unexpected as other antibodies that have been tested that target IGF-1R with the YTE or LS mutations had similar expressions regardless of the Fc mutations. The YTE version had fewer lower molecular weight species as compared to the LS version. Thus, indicating that the YTE antibody has fewer impurities and is a more homogenous composition, which provides advantages over the antibody with the LS mutation. This was also not predictable as another antibody that was evaluated showed the opposite effect on such species. Furthermore, during purification, it was found that the LS mutant formed more aggregates when being purified on a cation exchange column as compared VRDN-2700. The aggregation of the LS mutant would cause significant manufacturing issues, which were not observed for VRDN-2700. Therefore, this difference in the Fc mutants for this antibody could not have been predicted or expected and leads to significant and unexpected advantages for the antibody that is referenced herein as VRDN-2700.

The prolonged half-life of VRDN-2700 (YTE) demonstrates that it can be used in a convenient SC injection, or as an IV infusion requiring fewer and/or less frequent treatments vs. conventional therapeutic IgG antibodies and has superior properties as compared to other Fc mutant versions of the same antibody (same variable regions).

### Example 5: Teprotumumab and Variant IGF-1R Antibodies Block IGF-1 Stimulation (Prophetic)

Blockage of IGF-1 stimulation is measured by secretion of hyaluronan, in the presence of IGF-1R antibodies with VH and VL sequences of SEQ ID NOs: 123-262, all of which are disclosed herein. Immunoglobulins are purified from the sera of patients with Graves' ophthalmopathy (GO) and tested for their ability to activate TSHR and/or IGF-1R directly, and TSHR/IGF-1R cross talk in primary cultures of GO fibroblasts. Cells are treated with M22 or GO-Igs with or without IGF-1R inhibitory antibodies such as those provided for herein, including but not limited to antibodies with VH and VL sequences of SEQ ID NOs: 123-262, all of which are disclosed herein. Hyaluronan (hyaluronic acid; HA) secretion is measured as a major biological response for GO fibroblast stimulation. IGF-1R autophosphorylation is used as a measure of direct IGF-1R activation. TSHR activation is determined through cyclic-AMP (cAMP) production. The IGF-1R antibodies, as disclosed herein, are found to effectively block HA secretion and, therefore, are found to block IGF stimulation.

### Example 6: Treatment of Patients with Thyroid Eye Disease and Clinical Assessment of IGF-1R Antibodies on Thyroid Eye Disease (Prophetic)

Infusions of IGF-1R inhibitory antibodies such as those provided for herein, including but not limited to antibodies with VH and VL sequences of SEQ ID NOs: 123-262, all of which are disclosed herein, are provided to the subjects. The number of infusions is individualized for each subject and is based on the investigator's clinical judgment. The Day 1 Visit occurs within 14 days after the final visit of the prior trial. Visit windows are ±1 day for Weeks 1 and 4, ±3 days for Weeks 3, 6, 9, 12, 15, 18, 21, and 24. The Follow-up period is meant for subjects who were proptosis non-responders in the prior trial only; subjects who relapsed in the prior trial did not participate in the Follow-Up Period. Visit windows during the Follow-up period are ±7 days.

Treatment Period is 24 weeks (6 months), during which 8 infusions of teprotumumab are administered.

Subjects who are proptosis non-responders are scheduled to participate in a 6-month Follow-Up Period in this extension study; subjects who relapsed in the lead-in study and are retreated in this extension study will not participate in the Follow-Up Period.

Efficacy assessments are performed for both eyes at each assessment time point. The “study eye” (i.e., the more severely affected eye) will remain the same as that identified at the Baseline (Day 1) Visit of the prior study. Both eyes are assessed for efficacy but the study eye is used to assess the primary outcome measure.

Efficacy is assessed by proptosis (measured as exophthalmos evaluation of the Clinical Measures of Severity using a Hertel instrument for consistency in measurement), CAS (7-item scale), diplopia (measured as part of the Clinical Measures of Severity) and Clinical Measures of Severity (including motility restriction assessments).

Quality of life is assessed using the GO-QoL questionnaire.

Safety is assessed via AE and concomitant medication use monitoring, immunogenicity testing, physical and ophthalmic examinations, vital signs, clinical safety laboratory evaluations (complete blood count, chemistry (including thyroid panel and HbA1C), and urinalysis), pregnancy testing (if applicable), and electrocardiograms (ECG). The study is also monitored by a Data Safety Monitoring Board (DSMB).

Proptosis assessments is performed using a Hertel exophthalmometer for consistency in measurement, and (except when strictly unavoidable) the same Hertel instrument and same observer is used at each evaluation for the full duration of the study. Additionally, the same intercanthal distance (ICD) is used on each occasion.

Proptosis is measured for each eye on Day 1 and Weeks 6, 12, 18, and 24 (or premature withdrawal (PW)) during the Treatment Period, and at Months 7, 9, and 12 (or PW) during the Follow-Up Period. Measurements is recorded on the Clinical Measures of Severity eCRF under exophthalmos.

The antibodies are found to be effective in treating thyroid eye disease and also improving quality of life as provided for herein.

### Example 7: Antibody with Increased pK (Prophetic)

Cynomolgus monkeys are dosed with antibodies with VH and VL sequences of SEQ ID NOs: 124-262, all of which are disclosed herein, in an amount of 10 mg/kg by either intravenous or subcutaneous route, and samples are collected at 0.5 hr, 2 hr, 8 hr and days 1, 3, 7, 10, 14, 21, and 28 time points for PK analysis by ELISA. VRDN-02700 and SEQ ID NO: 123 are also administered at 10 mg/kg IV as comparators. The antibodies comprising VH and VL sequences of SEQ ID NOs: 124-262 are shown to have significantly higher PK as compared to Teprotumumab (SEQ ID NO: 123).

### Example 8: IGF-1R Antibody Binding Affinity

IGF-1R antibody binding affinities were determined by surface plasmon resonance (SPR). SPR measurements were performed on a Biacore 8K+ instrument (Cytiva) at 25° C. In all Biacore experiments HBS-EP+ (Cytiva) and NaOH 10 mM served as running buffer and regeneration buffer respectively. An anti-hulgG (Fcγ specific) monoclonal antibody was immobilized on a CM5 Series S Biacore chip for antibody capture. The immobilization was performed in accordance with a method provided by Biacore using an amine coupling kit. After activation of the chip, the capture antibody was injected resulting in a surface density of approximately 10,000 resonance units (RU). Anti-7GF-1R antibodies was injected at a concentration of 10 nM diluted in 1×HBS-EP+ assay buffer for one minute at a flow-rate of 30 μL/min followed by a stabilization period of one minute. Recombinant human his-tagged IGF 1R extracellular domain protein association was determined by injecting five consecutive concentrations, ranging from 6.3 to 100 nM, in two-fold serial dilutions in 1×HBS-EP+ pH 7.4 for one minute at 30 μL/min for single cycle analysis. Dissociation was subsequently monitored for ten minutes in HBS pH 7.4 or pH 6 buffer. The flow cell surfaces were regenerated by a double injection of 1 mM glycine at pH 1.5 at 30 μL/min. Kinetic parameters analysis was performed using the Biacore Insight Evaluation software (Cytiva) and using a 1:1 binding kinetic fitting model. The results are shown in the table below:

| | SPR at pH 7.4 | | | SPR at pH 6 | | |
|---|---|---|---|---|---|---|
| Antibody ID | ka (1/Ms) | kd (1/s) | KD (M) | ka (1/Ms) | kd (1/s) | KD (M) |
| VRDN-03100 | 1.07E+05 | 1.67E−04 | 1.55E−09 | 9.02E+04 | 7.21E−05 | 7.99E−10 |
| VRDN-03001 | 1.01E+05 | 2.14E−04 | 2.12E−09 | 9.17E+04 | 1.52E−04 | 1.65E−09 |
| VRDN-03002 | 9.58E+04 | 2.25E−04 | 2.34E−09 | 7.90E+04 | 1.31E−04 | 1.66E−09 |
| VRDN-03003 | 1.03E+05 | 2.04E−04 | 1.99E−09 | 8.93E+04 | 1.25E−04 | 1.40E−09 |
| VRDN-03004 | 1.02E+05 | 1.80E−04 | 1.76E−09 | 8.28E+04 | 7.96E−05 | 9.62E−10 |
| VRDN-03005 | 8.81E+04 | 2.78E−04 | 3.15E−09 | 7.85E+04 | 2.11E−04 | 2.69E−09 |
| VRDN-03006 | 9.83E+04 | 9.72E−05 | 9.88E−10 | 8.49E+04 | 6.31E−05 | 7.43E−10 |
| VRDN-03007 | 8.62E+04 | 2.19E−04 | 2.54E−09 | 8.19E+04 | 2.05E−04 | 2.50E−09 |
| VRDN-03008 | 1.02E+05 | 2.39E−04 | 2.36E−09 | 8.76E+04 | 1.38E−04 | 1.58E−09 |
| VRDN-03009 | 1.03E+05 | 1.72E−04 | 1.66E−09 | 9.57E+04 | 1.23E−04 | 1.28E−09 |
| VRDN-03010 | 1.07E+05 | 1.33E−04 | 1.25E−09 | 7.98E+04 | 4.67E−05 | 5.86E−10 |
| VRDN-03011 | 9.68E+04 | 2.95E−04 | 3.04E−09 | 8.49E+04 | 2.49E−04 | 2.93E−09 |
| VRDN-03012 | 5.70E+05 | 1.27E−02 | 2.22E−08 | 1.08E+06 | 1.36E−02 | 1.25E−08 |
| VRDN-03013 | 1.60E+06 | 2.82E−01 | 1.76E−07 | 1.91E+06 | 2.62E−02 | 1.37E−08 |
| VRDN-03014 | 9.21E+04 | 2.85E−04 | 3.09E−09 | 8.46E+04 | 3.81E−04 | 4.50E−09 |
| VRDN-03015 | 9.63E+04 | 2.53E−04 | 2.63E−09 | 7.45E+04 | 1.96E−04 | 2.64E−09 |
| VRDN-03016 | 1.86E+07 | 5.26E−01 | 2.83E−08 | 1.79E+05 | 3.22E−03 | 1.80E−08 |
| VRDN-03017 | 1.13E+05 | 8.42E−04 | 7.43E−09 | 5.57E+04 | 8.19E−04 | 1.47E−08 |
| VRDN-03018 | 4.09E+05 | 6.57E−03 | 1.61E−08 | 4.58E+05 | 5.67E−03 | 1.24E−08 |
| VRDN-03019 | 5.39E+06 | 1.57E−01 | 2.91E−08 | 2.01E+05 | 3.04E−03 | 1.52E−08 |
| VRDN-03020 | 2.13E+05 | 1.25E−04 | 5.88E−10 | 1.63E+05 | 3.43E−05 | 2.10E−10 |
| VRDN-03021 | 1.22E+09 | 3.16E+01 | 2.59E−08 | 2.06E+05 | 3.16E−03 | 1.53E−08 |
| VRDN-03022 | 2.27E+05 | 9.21E−05 | 4.05E−10 | 2.03E+05 | 1.10E−04 | 5.44E−10 |
| VRDN-03023 | 1.06E+05 | 1.57E−04 | 1.48E−09 | 8.13E+04 | 8.22E−05 | 1.01E−09 |
| VRDN-03024 | 9.60E+04 | 2.64E−04 | 2.75E−09 | 6.90E+04 | 3.09E−04 | 4.48E−09 |
| VRDN-03025 | 1.08E+05 | 1.64E−04 | 1.52E−09 | 9.59E+04 | 1.27E−04 | 1.32E−09 |
| VRDN-03026 | 1.59E+05 | 9.11E−05 | 5.72E−10 | 1.37E+05 | 5.06E−05 | 3.68E−10 |
| VRDN-03027 | 1.59E+05 | 1.22E−04 | 7.66E−10 | 1.27E+05 | 3.53E−05 | 2.77E−10 |
| VRDN-03028 | 2.46E+05 | 4.60E−05 | 1.87E−10 | 1.94E+05 | 1.98E−05 | 1.02E−10 |
| VRDN-03029 | 2.36E+06 | 6.90E−02 | 2.93E−08 | 1.47E+06 | 1.61E−02 | 1.10E−08 |
| VRDN-03030 | 8.47E+04 | 4.36E−04 | 5.15E−09 | 6.32E+04 | 3.53E−04 | 5.58E−09 |
| VRDN-03031 | 9.44E+04 | 3.38E−04 | 3.58E−09 | 8.31E+04 | 1.31E−04 | 1.58E−09 |
| VRDN-03032 | 8.68E+05 | 1.11E−01 | 1.28E−07 | 4.06E+05 | 6.50E−03 | 1.60E−08 |
| VRDN-03033 | NA | NA | NA | 2.55E+05 | 2.50E−03 | 9.80E−09 |
| VRDN-03034 | 8.20E+04 | 5.70E−04 | 6.95E−09 | 3.57E+04 | 6.80E−04 | 1.91E−08 |
| VRDN-03035 | 2.95E+05 | 3.51E−03 | 1.19E−08 | 2.21E+05 | 2.64E−03 | 1.19E−08 |
| VRDN-03036 | 1.04E+05 | 7.07E−04 | 6.80E−09 | 6.96E+04 | 6.85E−04 | 9.84E−09 |
| VRDN-03037 | 1.02E+05 | 1.93E−04 | 1.89E−09 | 9.12E+04 | 1.26E−04 | 1.39E−09 |
| VRDN-03038 | 9.69E+04 | 1.67E−04 | 1.72E−09 | 8.20E+04 | 1.15E−04 | 1.40E−09 |
| VRDN-03039 | 2.94E+05 | 3.32E−03 | 1.13E−08 | 3.94E+05 | 5.63E−03 | 1.43E−08 |
| VRDN-03040 | 1.05E+05 | 2.24E−04 | 2.14E−09 | 9.12E+04 | 1.83E−04 | 2.01E−09 |
| VRDN-03041 | 9.49E+04 | 3.00E−04 | 3.16E−09 | 6.50E+04 | 3.61E−05 | 5.56E−10 |
| VRDN-03042 | 1.12E+05 | 1.72E−04 | 1.53E−09 | 7.84E+04 | 9.44E−05 | 1.20E−09 |
| VRDN-03043 | 4.93E+05 | 1.11E−02 | 2.24E−08 | 1.39E+06 | 2.53E−02 | 1.82E−08 |
| VRDN-03044 | 9.84E+04 | 1.92E−04 | 1.96E−09 | 5.73E+04 | 2.17E−04 | 3.79E−09 |
| VRDN-03045 | 1.05E+05 | 1.35E−04 | 1.29E−09 | 8.05E+04 | 5.02E−05 | 6.24E−10 |
| VRDN-03046 | 1.03E+05 | 1.62E−04 | 1.58E−09 | 8.48E+04 | 1.59E−04 | 1.87E−09 |
| VRDN-03047 | 9.57E+04 | 2.82E−04 | 2.95E−09 | 7.81E+04 | 2.94E−04 | 3.77E−09 |
| VRDN-03048 | NA | NA | NA | NA | NA | NA |
| VRDN-03049 | 1.17E+05 | 1.83E−04 | 1.56E−09 | 7.43E+04 | 2.01E−04 | 2.70E−09 |
| VRDN-03050 | 1.03E+05 | 2.10E−04 | 2.03E−09 | 7.58E+04 | 1.70E−04 | 2.25E−09 |
| VRDN-03051 | 1.10E+05 | 2.03E−04 | 1.86E−09 | 7.62E+04 | 1.62E−04 | 2.12E−09 |
| VRDN-03052 | 8.93E+04 | 2.43E−04 | 2.72E−09 | 8.53E+04 | 1.27E−04 | 1.49E−09 |
| VRDN-03053 | 1.05E+05 | 1.88E−04 | 1.79E−09 | 9.55E+04 | 1.60E−04 | 1.67E−09 |
| VRDN-03054 | 9.35E+04 | 1.04E−04 | 1.11E−09 | 9.45E+04 | 8.18E−05 | 8.66E−10 |
| VRDN-03055 | 9.77E+04 | 1.88E−04 | 1.92E−09 | 8.70E+04 | 1.40E−04 | 1.61E−09 |
| VRDN-03056 | NA | NA | NA | NA | NA | NA |
| VRDN-03057 | 1.04E+05 | 1.07E−03 | 1.03E−08 | 6.57E+04 | 1.25E−03 | 1.90E−08 |
| VRDN-03058 | NA | NA | NA | NA | NA | NA |
| VRDN-03059 | 1.05E+05 | 6.01E−04 | 5.74E−09 | 8.44E+04 | 6.88E−04 | 8.15E−09 |
| VRDN-03060 | 1.27E+05 | 1.04E−03 | 8.16E−09 | 7.90E+04 | 2.56E−04 | 3.25E−09 |
| VRDN-03061 | 9.23E+04 | 3.95E−04 | 4.28E−09 | 6.85E+04 | 1.01E−03 | 1.47E−08 |
| VRDN-03062 | NA | NA | NA | NA | NA | NA |
| VRDN-03063 | 8.81E+04 | 7.02E−04 | 7.96E−09 | 4.39E+04 | 8.92E−04 | 2.03E−08 |
| VRDN-03064 | 2.59E+05 | 2.32E−03 | 8.94E−09 | 1.92E+05 | 1.73E−03 | 8.99E−09 |
| VRDN-03065 | 1.20E+05 | 8.29E−04 | 6.90E−09 | 2.83E+04 | 9.09E−04 | 3.21E−08 |
| VRDN-03066 | 1.59E+05 | 3.55E−04 | 2.24E−09 | NA | NA | NA |
| VRDN-03067 | 1.83E+05 | 2.87E−03 | 1.57E−08 | NA | NA | NA |
| VRDN-03068 | 9.81E+04 | 1.49E−03 | 1.52E−08 | NA | NA | NA |
| VRDN-03069 | 1.65E+05 | 2.10E−04 | 1.27E−09 | NA | NA | NA |
| VRDN-03070 | 4.26E+04 | 1.82E−04 | 4.28E−09 | NA | NA | NA |
| VRDN-03071 | 9.90E+04 | 1.42E−03 | 1.43E−08 | NA | NA | NA |
| VRDN-03072 | 5.86E+04 | 2.17E−04 | 3.70E−09 | NA | NA | NA |
| VRDN-03073 | 2.16E+05 | 2.57E−03 | 1.19E−08 | NA | NA | NA |
| VRDN-03074 | 2.29E+05 | 3.98E−03 | 1.74E−08 | NA | NA | NA |
| VRDN-03075 | 1.45E+05 | 1.04E−04 | 7.17E−10 | NA | NA | NA |
| VRDN-03076 | 1.49E+05 | 9.79E−05 | 6.55E−10 | NA | NA | NA |
| VRDN-03077 | 1.45E+05 | 5.85E−05 | 4.04E−10 | NA | NA | NA |
| VRDN-03078 | 1.02E+05 | 7.12E−05 | 6.99E−10 | NA | NA | NA |
| VRDN-03079 | 7.69E+04 | 3.85E−04 | 5.00E−09 | NA | NA | NA |
| VRDN-03080 | 7.92E+04 | 1.66E−04 | 2.10E−09 | NA | NA | NA |
| VRDN-03081 | 1.03E+05 | 3.25E−04 | 3.17E−09 | NA | NA | NA |
| VRDN-03082 | 1.35E+05 | 6.29E−04 | 4.65E−09 | NA | NA | NA |
| VRDN-03083 | 1.44E+05 | 4.20E−04 | 2.92E−09 | NA | NA | NA |
| VRDN-03084 | 1.39E+05 | 9.50E−04 | 6.84E−09 | NA | NA | NA |
| VRDN-03085 | 2.27E+05 | 2.57E−03 | 1.13E−08 | NA | NA | NA |
| VRDN-03086 | 1.05E+05 | 8.47E−04 | 8.09E−09 | NA | NA | NA |
| VRDN-03087 | 1.21E+05 | 1.02E−04 | 8.44E−10 | NA | NA | NA |
| VRDN-03088 | 1.40E+05 | 1.35E−03 | 9.60E−09 | NA | NA | NA |
| VRDN-03089 | 7.33E+04 | 1.73E−02 | 2.36E−07 | NA | NA | NA |
| VRDN-03090 | 9.07E+04 | 1.60E−04 | 1.77E−09 | NA | NA | NA |
| VRDN-03091 | 8.19E+05 | 9.02E−03 | 1.10E−08 | NA | NA | NA |
| VRDN-03092 | NA | NA | NA | NA | NA | NA |
| VRDN-03093 | 4.93E+04 | 1.88E−04 | 3.81E−09 | NA | NA | NA |
| VRDN-03094 | 5.72E+04 | 3.41E−06 | 5.97E−11 | NA | NA | NA |
| VRDN-03095 | 5.32E+04 | 6.70E−05 | 1.26E−09 | NA | NA | NA |
| VRDN-03096 | 4.68E+04 | 2.40E−04 | 5.12E−09 | NA | NA | NA |
| VRDN-03097 | 6.06E+04 | 1.55E−04 | 2.56E−09 | NA | NA | NA |
| VRDN-03098 | 6.59E+04 | 1.35E−04 | 2.05E−09 | 2.47E+05 | 3.06E−04 | 1.24E−09 |
| VRDN-03099 | 6.37E+04 | 1.39E−04 | 2.18E−09 | 2.21E+05 | 2.96E−04 | 1.34E−09 |
| VRDN-03100 | 2.44E+05 | 1.34E−03 | 5.52E−09 | 6.30E+05 | 1.07E−03 | 1.69E−09 |
| VRDN-03101 | 8.93E+04 | 7.02E−05 | 7.85E−10 | 2.19E+05 | 2.68E−04 | 1.23E−09 |
| VRDN-03102 | 6.45E+04 | 1.73E−04 | 2.68E−09 | 2.18E+05 | 3.19E−04 | 1.46E−09 |
| VRDN-03103 | 4.98E+05 | 6.37E−03 | 1.28E−08 | 1.50E+06 | 2.16E−03 | 1.44E−09 |
| VRDN-03104 | 8.50E+04 | 2.59E−04 | 3.05E−09 | 1.73E+05 | 2.83E−04 | 1.63E−09 |
| VRDN-03105 | 4.05E+05 | 3.61E−04 | 8.90E−10 | 1.41E+05 | 2.81E−04 | 1.99E−09 |
| VRDN-03106 | 6.51E+04 | 1.05E−03 | 1.61E−08 | 1.20E+06 | 2.18E−03 | 1.81E−09 |
| VRDN-03107 | 5.80E+04 | 1.56E−04 | 2.69E−09 | 2.42E+05 | 3.44E−04 | 1.42E−09 |
| VRDN-03108 | 4.90E+04 | 1.95E−04 | 3.97E−09 | 2.24E+05 | 4.13E−04 | 1.85E−09 |
| VRDN-03109 | 4.25E+04 | 2.86E−04 | 6.74E−09 | 1.80E+05 | 4.90E−04 | 2.72E−09 |
| VRDN-03110 | 6.05E+04 | 2.09E−04 | 3.45E−09 | 2.23E+05 | 3.78E−04 | 1.70E−09 |
| VRDN-03111 | 7.70E+04 | 3.62E−04 | 4.71E−09 | 2.33E+05 | 4.79E−04 | 2.06E−09 |
| VRDN-03112 | 1.09E+05 | 3.22E−04 | 2.97E−09 | 1.69E+05 | 4.63E−04 | 2.74E−09 |
| VRDN-03113 | 1.41E+06 | 3.88E−03 | 2.76E−09 | 7.71E+05 | 1.91E−03 | 2.48E−09 |
| VRDN-03114 | 2.97E+04 | 9.91E−04 | 3.34E−08 | 1.22E+05 | 6.90E−06 | 5.65E−11 |
| VRDN-03115 | NA | NA | NA | 2.46E+05 | 2.97E−04 | 1.21E−09 |
| VRDN-03116 | 2.12E+05 | 9.10E−04 | 4.30E−09 | NA | NA | NA |
| VRDN-03117 | 4.83E+05 | 3.91E−03 | 8.10E−09 | NA | NA | NA |
| VRDN-03118 | 9.86E+04 | 1.07E−04 | 1.08E−09 | NA | NA | NA |
| VRDN-03119 | 1.57E+05 | 1.53E−03 | 9.78E−09 | NA | NA | NA |
| VRDN-03120 | 3.22E+05 | 1.28E−03 | 3.96E−09 | NA | NA | NA |
| VRDN-03121 | 1.14E+05 | 4.86E−04 | 4.27E−09 | NA | NA | NA |
| VRDN-03122 | 1.11E+05 | 4.55E−04 | 4.09E−09 | NA | NA | NA |
| VRDN-03123 | 6.42E+04 | 9.19E−05 | 1.43E−09 | NA | NA | NA |
| VRDN-03124 | 8.52E+04 | 1.57E−04 | 1.84E−09 | NA | NA | NA |
| VRDN-03125 | 3.39E+05 | 2.92E−03 | 8.61E−09 | NA | NA | NA |
| VRDN-03126 | 3.41E+05 | 2.97E−03 | 8.70E−09 | NA | NA | NA |
| VRDN-03127 | 1.31E+05 | 7.48E−05 | 5.73E−10 | NA | NA | NA |
| VRDN-03128 | 3.31E+05 | 2.41E−03 | 7.30E−09 | NA | NA | NA |
| VRDN-03129 | 6.46E+05 | 7.09E−03 | 1.10E−08 | NA | NA | NA |
| VRDN-03130 | 1.01E+05 | 2.75E−04 | 2.72E−09 | NA | NA | NA |
| VRDN-03131 | 8.69E+04 | 1.41E−03 | 1.62E−08 | NA | NA | NA |
| VRDN-03132 | 3.95E+05 | 4.00E−03 | 1.01E−08 | NA | NA | NA |
| VRDN-03133 | 3.50E+05 | 2.28E−03 | 6.51E−09 | NA | NA | NA |
| VRDN-03134 | 2.45E+05 | 1.20E−03 | 4.89E−09 | NA | NA | NA |
| VRDN-03135 | 1.02E+05 | 4.14E−04 | 4.06E−09 | NA | NA | NA |
| VRDN-03136 | 8.91E+04 | 2.53E−04 | 2.84E−09 | NA | NA | NA |
| VRDN-03137 | 9.40E+05 | 1.42E−02 | 1.51E−08 | NA | NA | NA |
| VRDN-03138 | 4.95E+05 | 4.33E−03 | 8.75E−09 | NA | NA | NA |
| VRDN-03139 | 1.07E+05 | 2.62E−04 | 2.46E−09 | NA | NA | NA |

Each of these examples and the embodiments provided herein demonstrate that the antibodies provided for herein can be used to treat TED and their associate symptoms.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present embodiments are not to be limited in scope by the specific embodiments described herein. Indeed, various modifications in addition to those described herein will become apparent to those skilled in the art from the foregoing description. Such modifications are intended to fall within the scope of the embodiments and any appended claims.

The present specification is considered to be sufficient to enable one skilled in the art to practice the embodiments. Various modifications in addition to those shown and described herein will become apparent to those skilled in the art from the foregoing description and fall within the scope of the present disclosure and any appended claims.

What is claimed is:

1. An antibody, or antigen binding fragment thereof, comprising:
a heavy chain complementarity-determining region (HCDR) sequence selected from the group consisting of SEQ ID NO: 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, and 321; and
a light chain CDR (LCDR) sequence selected from the group consisting of SEQ ID NO: 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, and 362.

2. An antibody, or antigen binding fragment thereof, comprising:
a heavy chain variable region (VH) sequence selected from the group consisting of SEQ ID NO: 1, 3, 4, 5, 6, 7, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 67, 68, 69, 70, 71, 72, 73, 74, 75, 76, 77, 78, 79, 80, 81, 82, 83, 84, 85, 86, 87, 88, 99, 101, 102, 103, 104, 105, 106, 107, 108, 110, 111, 112, 113, 114, 115, 116, 117, 118, 119, 120, and 121; and
a light chain variable region (VL) sequence selected from the group consisting of SEQ ID NO: 2, 8, 39, 40, 41, 42, 43, 44, 45, 46, 47, 48, 49, 50, 51, 52, 53, 54, 55, 56, 57, 58, 59, 60, 61, 62, 63, 64, 65, 66, 89, 90, 91, 92, 93, 94, 95, 96, 97, 98, 100, 109, and 122.

3. The antibody of any one of claims 1 or 2, or antigen binding fragment thereof, wherein the antibody binds to insulin-like growth factor I receptor (IGF-1R).

4. The antibody of any one of claims 1-3, wherein the antibody is a monoclonal antibody.

5. The antibody of any one of claims 1-4, wherein the antibody is a humanized antibody.

6. The antibody of any one of claims 1-5, wherein the antibody is a scFv antibody.

7. The antibody of any one of claims 1-6, wherein the antibody, or antigen binding fragment thereof, comprises a VH peptide comprising an amino acid sequence as set forth in SEQ ID NOs: 1, 3, 4, 5, 6, 7, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 67, 68, 69, 70, 71, 72, 73, 74, 75, 76, 77, 78, 79, 80, 81, 82, 83, 84, 85, 86, 87, 88, 99, 101, 102, 103, 104, 105, 106, 107, 108, 110, 111, 112, 113, 114, 115, 116, 117, 118, 119, 120, or 121, or any variant thereof.

8. The antibody of any one of claims 1-7, wherein the antibody, or antigen binding fragment thereof, comprises a VL peptide comprising an amino acid sequence as set forth in SEQ ID NOs: 2, 8, 39, 40, 41, 42, 43, 44, 45, 46, 47, 48, 49, 50, 51, 52, 53, 54, 55, 56, 57, 58, 59, 60, 61, 62, 63, 64, 65, 66, 89, 90, 91, 92, 93, 94, 95, 96, 97, 98, 100, 109, or 122, or any variant thereof.

9. An antibody, or antigen binding fragment thereof, wherein the antibody or antibody fragment comprises: (i) a heavy chain variable region comprising HCDR1, HCDR2, and HCDR3 sequences, wherein the HCDR1 sequence has the amino acid sequence of SEQ ID NO: 263, 264, 265, 266, 267, 268, 269, 270, 271, 272, 273, 274, 275, or 276; the HCDR2 has the amino acid sequence of SEQ ID NO: 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, or 397; and the HCDR3 sequence has the amino acid sequence of SEQ ID NO: 311, 312, 313, 314, 315, 316, 317, 318, 319, 320, or 321; or variants of any of the foregoing; and (ii) a light chain variable region comprising LCDR1, LCDR2, and LCDR3 sequences, wherein the LCDR1 sequence has the amino acid sequence SEQ ID NO: 322, 323, 324, 325, 326, 327, 328, 329, 330, 331, 332, 333, 334, 335, 336, 337, or 338; the LCDR2 sequence has the amino acid sequence of SEQ ID NO: 339, 340, 341, 342, 343, 344, 345, 346, 347, 348, 349, or 350; and the LCDR3 sequence has the amino acid sequence of SEQ ID NO: 351, 352, 353, 354, 355, 356, 357, 358, 359, 360, 361, or 362; or variants of any of the foregoing.

10. An antibody, or antigen binding fragment thereof, comprising a VH and VL pair comprising the amino acid sequence of SEQ ID NOs:123-262, or a variant thereof.

11. The antibody of any one of claims 1-10, wherein the heavy chain variable region and the light chain variable region are not linked by a linker.

12. The antibody of any one of claims 1-10, wherein the heavy chain variable region and the light chain variable region are linked with a peptide linker.

13. The antibody of claim 12, wherein the peptide linker comprises a sequence of: (GGGGS)ₙ (SEQ ID NO: 375) (GGGGA)ₙ (SEQ ID NO: 376), or any combination thereof, wherein each n is independently 1-5.

14. The antibody of any one of claims 1-13, wherein the variant has 1-10 substitutions, deletions, or insertions.

15. The antibody of any one of claims 1-14, wherein the variant has 1-10 conservative substitutions.

16. The antibody of any one of claims 1-15, wherein the variant has at least 85%, 90%, 91%, 92%, 93%, 94%, 95%, 96%, 97%, 98%, or 99% homology to a sequence of SEQ ID NOs: 1-122.

17. The antibody of any one of claims 1-15, wherein the variant has at least 85%, 90%, 91%, 92%, 93%, 94%, 95%, 96%, 97%, 98%, or 99% homology to a sequence of SEQ ID NOs: 123-262.

18. The antibody of any one of claims 1-15, wherein the variant has at least 85%, 90%, 91%, 92%, 93%, 94%, 95%, 96%, 97%, 98%, or 99% identify to a sequence of SEQ ID NO: 263-362, and 397.

19. The antibody of any one of claims 1-18, wherein the antibody is a scFv antibody.

20. The antibody of any one of claims 1-19, wherein the antibody is a monoclonal antibody.

21. The antibody of any one of claims 1-20, wherein the antibody is a humanized antibody.

22. The antibody of any one of claims 1-21, wherein the antibody comprises a Fc region.

23. The antibody of claim 22, wherein the Fc region is selected from the group consisting of SEQ ID NO: 363, 364, 365, 366, 367, 368, 369, and 370.

24. The antibody of any one of claims 1-23, wherein the Fc region comprises a mutation that extends the half-life of the antibody when linked to the Fc region.

25. The antibody of claim 24, wherein the Fc region comprises a S228P, L235E, M252Y, S254T, T256E, M428L, N434S, L234F, P331S mutation, or any combination thereof.

26. The antibody of claim 24, wherein the Fc region comprises a M252Y, S254T, and T256E mutation.

27. The antibody of claim 24, wherein the Fc region comprises a S228P and a L235E mutation.

28. The antibody of claim 24, wherein the Fc region comprises a L234F, L235E, and P331S mutation.

29. The antibody of claim 24, wherein the Fc region comprises M252Y, S254T, T256E, S228P and L235E mutations.

30. The antibody of claim 24, wherein the Fc region comprises S228P, L235E, M428L, and N434S mutations.

31. The antibody of claim 24, wherein the Fc region comprises M428L and N434S mutations.

32. The antibody of claim 24, wherein the Fc region comprises L234F, L235E, P331S, M252Y, S254T, and T256E mutations.

33. The antibody of any one of claims 1-32, wherein the antibody is an isolated antibody.

34. A nucleic acid molecule encoding an antibody, or antigen binding fragment thereof, of any one of claims 1-33.

35. A vector comprising the nucleic acid molecule of claim 34.

36. A cell comprising the nucleic comprising the nucleic acid molecule of claim 34 or the vector of claim 35.

37. A pharmaceutical composition comprising the antibody of any one of claims 1-33 or a nucleic acid molecule encoding the same.

38. The pharmaceutical composition of claim 37, wherein the composition is an injectable pharmaceutical composition.

39. The pharmaceutical composition of claims 37 or 38, wherein the pharmaceutically composition is administered intravenously or subcutaneously.

40. A method of treating or reducing the severity of, thyroid-associated ophthalmopathy (TAO), or a symptom thereof, comprising administering to a subject an antibody of any one of claims 1-33 or a pharmaceutical composition of any one of claims 37-39.

41. A method of reducing proptosis in an eye in a subject with thyroid-associated ophthalmopathy (TAO) comprising administering to a subject an antibody of any one of claims 1-33 or a pharmaceutical composition of any one of claims 37-39.

42. A method of treating thyroid eye disease in a subject comprising administering to a subject an antibody of any one of claims 1-33 or a pharmaceutical composition of any one of claims 37-39.

43. A method of reducing Clinical Activity Score (CAS) of thyroid-associated ophthalmopathy (TAO) in a subject comprising administering to a subject an antibody of any one of claims 1-33 or a pharmaceutical composition of any one of claims 37-39.

44. A method of a) reducing proptosis by at least 2 mm and b) reducing the clinical activity score (CAS) in a subject with thyroid-associated ophthalmopathy (TAO) comprising administering to a subject an antibody of any one of claims 1-33 or a pharmaceutical composition of any one of claims 37-39.

45. The method of any of claims 40-44, wherein proptosis is reduced by at least 2 mm.

46. The method of any of claims 40-44, wherein proptosis is reduced by at least 3 mm.

47. The method of any of claims 40-44, wherein proptosis is reduced by at least 4 mm.

48. The method of any of claims 40-44, wherein the clinical activity score (CAS) of the subject is reduced by at least 2 points.

49. The method of any of claims 40-44, wherein the clinical activity score (CAS) of the subject is reduced to one (1).

50. The method of any of claims 40-44, wherein the clinical activity score (CAS) of the subject is reduced to zero (0).

51. A method of treating or reducing the severity of thyroid-associated ophthalmopathy (TAO) in a subject comprising administering to a subject an antibody of any one of claims 1-33 or a pharmaceutical composition of any one of claims 36-38, wherein treatment with said antibody (i) reduces proptosis by at least 2 mm in an eye; (ii) is not accompanied by a deterioration of 2 mm or more in the other (or fellow eye); and (iii) reduces the CAS in said subject to either one (1) or zero (0).

52. A method of improving the quality of life in a subject with thyroid-associated ophthalmopathy (TAO, also called Graves' Ophthalmopathy/Graves' Orbitopathy) comprising administering to a subject an antibody of any one of claims 1-33 or a pharmaceutical composition of any one of claims 37-39.

53. The method of claim 52, wherein the quality of life is measured by the Graves' Ophthalmopathy Quality of Life (GO-QoL) assessment, or either the Visual Functioning or Appearance subscale thereof.

54. The method of claim 53, wherein the treatment results in an improvement of greater than or equal to 8 points on the GO-QoL.

55. The method of claim 53, wherein the treatment results in an improvement on the Functioning subscale of the GO-QoL.

56. The method of claim 53, wherein the treatment results in an improvement on the Appearance subscale of the GO-QoL.

57. A method of treating or reducing the severity of diplopia in a subject with thyroid-associated ophthalmopathy (TAO) comprising administering to a subject an antibody of any one of claims 1-33 or a pharmaceutical composition of any one of claims 37-39.

58. The method of claim 57, wherein the diplopia is constant diplopia.

59. The method of claim 57, wherein the diplopia is inconstant diplopia.

60. The method of claim 57, wherein the diplopia is intermittent diplopia.

61. The method of claim 57, wherein the improvement in or reduction in severity of diplopia is sustained at least 20 weeks after discontinuation of antibody administration.

62. The method of claim 57, wherein the improvement in or reduction in severity of diplopia is sustained at least 50 weeks after discontinuation of antibody administration.

63. The method of any one of claims 40-62, wherein said antibody is administered at a dosage of about 1 mg/kg to about 5 mg/kg antibody as a first dose.

64. The method of any one of claims 40-62, wherein said antibody is administered at a dosage of about 5 mg/kg to about 10 mg/kg antibody as a first dose.

65. The method of any one of claims 40-62, wherein said antibody is administered at a dosage of about 5 mg/kg to about 20 mg/kg antibody in subsequent doses.

66. The method of any one of claims 40-62, wherein said antibody is administered in the following amounts: about 10 mg/kg antibody as a first dose; and about 20 mg/kg antibody in subsequent doses.

67. The method of claim 66, wherein said subsequent doses are administered every three weeks for at least 21 weeks.

68. The method of any one of claims 40-67, wherein the antibody, or an antigen binding fragment thereof, is a human antibody, a monoclonal antibody, a human monoclonal antibody, a purified antibody, a diabody, a single-chain antibody, a multi-specific antibody, Fab, Fab′, F(ab′)2, Fv or scFv.

69. The method of any one of claims 40-68, wherein the antibody, or an antigen binding fragment thereof, is administered in a pharmaceutical composition that additionally comprises a pharmaceutically acceptable diluent or excipient or carrier.

70. The method of claim 69, wherein the pharmaceutical composition further comprises one or more pharmaceutically active compounds for the treatment of TAO.

71. The method of claim 69 or 70, wherein the pharmaceutical composition further comprises corticosteroids; rituximab or other anti-CD20 antibodies; tocilizumab or other anti-IL-6 antibodies; or selenium, infliximab or other anti-TNFalpha antibodies or a thyroid-stimulating hormone receptor (TSHR) inhibitor.

72. The method of any one of the claims 4-71, wherein the antibody or an antigen binding fragment thereof is administered directly to the eye, the anterior chamber of the eye, the vitreous chamber of the eye, the suprachoroidal space, or the retro-orbital sinus.

73. The method of claim 72, wherein the antibody or an antigen binding fragment thereof is administered via an injection.

74. The method of claim 73, wherein the injection is a intravitreal injection, intraorbital injection, retro-orbital injection, suprachoroidal injection, or intracameral injection.

75. A method of increasing the internalization of IGF-1R on a cell, the method comprising contacting the cell with an antibody of any one of claims 1-33 or a pharmaceutical composition of any one of claims 37-39.

76. The method of claim 74, wherein the contacting comprises administering to a subject an antibody of any one of claims 1-33 or a pharmaceutical composition of any one of claims 37-39.

77. The method of claim 76, wherein the subject has or is at risk of thyroid eye disease (TED).

78. A method of inhibiting IGF-1 stimulated receptor phosphorylation on a cell, the method comprising contacting the cell with an antibody of any one of claims 1-33 or a pharmaceutical composition of any one of claims 37-39.

79. The method of claim 78, wherein the contacting comprises administering to a subject an antibody of any one of claims 1-33 or a pharmaceutical composition comprising of any one of claims 36-38.

80. The method of claim 79, wherein the subject has or is at risk of thyroid eye disease (TED).

81. The method of any one of claims 78-80, wherein the antibody has an IC50 of less than, or equal to, about 0.2 nm, 0.15 nm, 0.10 nm, 0.09 nm.

82. The method of claim 81, wherein the IC50 is measured in an in vitro assay, such as an assay as provided for herein.

83. The method of any one of claims 78-82, wherein the cell is an A549 cell or a HOCF cell.

84. A method of treating thyroid eye disease in a subject, the method comprising administering an antibody of any one of claims 1-33, or a pharmaceutical composition of any one of claims 37-39 to the subject, wherein the antibody has a serum concentration in the subject of at least, or about, 70 μg/ml, 75 μg/ml, 80 μg/ml, 85 μg/ml, 90 μg/ml, 95 μg/ml, 100 μg/ml, or 105 μg/ml at least 1, 2, or 3 week after administration.

85. The method of claim 84, wherein the antibody or the pharmaceutical composition is administered intravenously.

86. The method of any one of claims 84-85, wherein the antibody or the pharmaceutical composition is administered at a dose of about 20 mg/kg.

87. The method of any one of claims 84-86, wherein the antibody or the pharmaceutical composition is administered at least, or about, once a week, once every two weeks, once every 3 weeks, or once every 4 weeks.

88. A method of inhibiting IGF-1 induced receptor autophosphorylation in a cell by at least 95%, 96%, 97%, 98%, or 99% or by 100%, the method comprising contacting the cell with an antibody of any one of claims 1-33, or a pharmaceutical composition of any one of claims 37-39.

89. The method of claim 88, wherein the inhibition of the IGF-1 induced receptor autophosphorylation is measured as compared to the induced receptor autophosphorylation in the absence of the antibody or the pharmaceutical composition.

90. The method of claims 88 or 89, wherein the contacting comprises administering to a subject the antibody or the pharmaceutical composition comprising the same.

91. The method of claim 90, wherein the subject has or is at risk of thyroid eye disease (TED).

92. A method of inhibiting IGF-1 induced receptor autophosphorylation by at least 95%, 96%, 97%, 98%, or 99% or by 100% in a subject in need thereof, the method comprising administering to the subject an antibody of any one of claims 1-33, or a pharmaceutical composition of any one of claims 37-39.

93. The method of claim 92, wherein the subject has or is at risk of thyroid eye disease (TED).

94. The method of any one of claims 92 or 93, wherein the antibody or the pharmaceutical composition is administered intravenously.

95. The method of any one of claims 88-94, wherein the antibody comprises the CDRs of VRDN-1100.

96. The method of any one of claims 88-95, wherein the antibody comprises the CDRs of the antibody of VRDN-1100 or the CDRs of VRDN-2700.


SEQUENCE LISTING (397 sequences)

SEQ ID NO: 1 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 2 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 3 - protein, 118 aa, synthetic construct
QVQLVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 4 - protein, 118 aa, synthetic construct
QVELVESGGGVVQPGRSLRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 5 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYYCARELGRRYFDLWGRGTLVSVSS

SEQ ID NO: 6 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVTVSS

SEQ ID NO: 7 - protein, 118 aa, synthetic construct
QVQLVESGGGVVQPGRSLRLSCAASGFTFSSYGMHWVRQAPGKGLEWVAIIWFDGSSTYYADSVRGRFTISRDNSKNTLYLQMNSLRAEDTAVYYCARELGRRYFDLWGRGTLVTVSS

SEQ ID NO: 8 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 9 - protein, 118 aa, synthetic construct
QVELVESGGGVVQPGRSQRLSCAASGFTFSHYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 10 - protein, 118 aa, synthetic construct
QVELVESGGGVVQPGRSQRLSCAASGFTFSSHGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 11 - protein, 118 aa, synthetic construct
QVELVESGGGVVQPGRSQRLSCAASGFTFSSYHMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 12 - protein, 118 aa, synthetic construct
QVELVESGGGVVQPGRSQRLSCAASGFTFSSYGHHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 13 - protein, 118 aa, synthetic construct
QVELVESGGGVVQPGRSQRLSCAASGFTFSSYGMHWVRQAPGKGLEWVAHIWFDGSSTYYADSVRGRFTISRDNSKNTLYLQMNSLRAEDTAVYFCARELGRRYFDLWGRGTLVSVSS

SEQ ID NO: 14 - protein, 118 aa, synthetic construct
QVELVESGGGVVQPGRSQRLSCAASGFTFSSYGMHWVRQAPGKGLEWVAIHWFDGSSTYYADSVRGRFTISRDNSKNTLYLQMNSLRAEDTAVYFCARELGRRYFDLWGRGTLVSVSS

SEQ ID NO: 15 - protein, 118 aa, synthetic construct
QVELVESGGGVVQPGRSQRLSCAASGFTFSSYGMHWVRQAPGKGLEWVAIIHFDGSSTYYADSVRGRFTISRDNSKNTLYLQMNSLRAEDTAVYFCARELGRRYFDLWGRGTLVSVSS

SEQ ID NO: 16 - protein, 118 aa, synthetic construct
QVELVESGGGVVQPGRSQRLSCAASGFTFSSYGMHWVRQAPGKGLEWVAIIWHDGSSTYYADSVRGRFTISRDNSKNTLYLQMNSLRAEDTAVYFCARELGRRYFDLWGRGTLVSVSS

SEQ ID NO: 17 - protein, 118 aa, synthetic construct
QVELVESGGGVVQPGRSQRLSCAASGFTFSSYGMHWVRQAPGKGLEWVAIIWFHGSSTYYADSVRGRFTISRDNSKNTLYLQMNSLRAEDTAVYFCARELGRRYFDLWGRGTLVSVSS

SEQ ID NO: 18 - protein, 118 aa, synthetic construct
QVELVESGGGVVQPGRSQRLSCAASGFTFSSYGMHWVRQAPGKGLEWVAIIWFDHSSTYYADSVRGRFTISRDNSKNTLYLQMNSLRAEDTAVYFCARELGRRYFDLWGRGTLVSVSS

SEQ ID NO: 19 - protein, 118 aa, synthetic construct
QVELVESGGGVVQPGRSQRLSCAASGFTFSSYGMHWVRQAPGKGLEWVAIIWFDGHSTYYADSVRGRFTISRDNSKNTLYLQMNSLRAEDTAVYFCARELGRRYFDLWGRGTLVSVSS

SEQ ID NO: 20 - protein, 118 aa, synthetic construct
QVELVESGGGVVQPGRSQRLSCAASGFTFSSYGMHWVRQAPGKGLEWVAIIWFDGSHTYYADSVRGRFTISRDNSKNTLYLQMNSLRAEDTAVYFCARELGRRYFDLWGRGTLVSVSS

SEQ ID NO: 21 - protein, 118 aa, synthetic construct
QVELVESGGGVVQPGRSQRLSCAASGFTFSSYGMHWVRQAPGKGLEWVAIIWFDGSSHYYADSVRGRFTISRDNSKNTLYLQMNSLRAEDTAVYFCARELGRRYFDLWGRGTLVSVSS

SEQ ID NO: 22 - protein, 118 aa, synthetic construct
QVELVESGGGVVQPGRSQRLSCAASGFTFSSYGMHWVRQAPGKGLEWVAIIWFDGSSTHYADSVRGRFTISRDNSKNTLYLQMNSLRAEDTAVYFCARELGRRYFDLWGRGTLVSVSS

SEQ ID NO: 23 - protein, 118 aa, synthetic construct
QVELVESGGGVVQPGRSQRLSCAASGFTFSSYGMHWVRQAPGKGLEWVAIIWFDGSSTYHADSVRGRFTISRDNSKNTLYLQMNSLRAEDTAVYFCARELGRRYFDLWGRGTLVSVSS

SEQ ID NO: 24 - protein, 118 aa, synthetic construct
QVELVESGGGVVQPGRSQRLSCAASGFTFSSYGMHWVRQAPGKGLEWVAIIWFDGSSTYYHDSVRGRFTISRDNSKNTLYLQMNSLRAEDTAVYFCARELGRRYFDLWGRGTLVSVSS

SEQ ID NO: 25 - protein, 118 aa, synthetic construct
QVELVESGGGVVQPGRSQRLSCAASGFTFSSYGMHWVRQAPGKGLEWVAIIWFDGSSTYYAHSVRGRFTISRDNSKNTLYLQMNSLRAEDTAVYFCARELGRRYFDLWGRGTLVSVSS

SEQ ID NO: 26 - protein, 118 aa, synthetic construct
QVELVESGGGVVQPGRSQRLSCAASGFTFSSYGMHWVRQAPGKGLEWVAIIWFDGSSTYYADHVRGRFTISRDNSKNTLYLQMNSLRAEDTAVYFCARELGRRYFDLWGRGTLVSVSS

SEQ ID NO: 27 - protein, 118 aa, synthetic construct
QVELVESGGGVVQPGRSQRLSCAASGFTFSSYGMHWVRQAPGKGLEWVAIIWFDGSSTYYADSHRGRFTISRDNSKNTLYLQMNSLRAEDTAVYFCARELGRRYFDLWGRGTLVSVSS

SEQ ID NO: 28 - protein, 118 aa, synthetic construct
QVELVESGGGVVQPGRSQRLSCAASGFTFSSYGMHWVRQAPGKGLEWVAIIWFDGSSTYYADSVHGRFTISRDNSKNTLYLQMNSLRAEDTAVYFCARELGRRYFDLWGRGTLVSVSS

SEQ ID NO: 29 - protein, 118 aa, synthetic construct
QVELVESGGGVVQPGRSQRLSCAASGFTFSSYGMHWVRQAPGKGLEWVAIIWFDGSSTYYADSVRHRFTISRDNSKNTLYLQMNSLRAEDTAVYFCARELGRRYFDLWGRGTLVSVSS

SEQ ID NO: 30 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARHLGRRYFDLWGRGTLVSVSS

SEQ ID NO: 31 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCAREHGRRYFDLWGRGTLVSVSS

SEQ ID NO: 32 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELHRRYFDLWGRGTLVSVSS

SEQ ID NO: 33 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGHRYFDLWGRGTLVSVSS

SEQ ID NO: 34 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRHYFDLWGRGTLVSVSS

SEQ ID NO: 35 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRHFDLWGRGTLVSVSS

SEQ ID NO: 36 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYHDLWGRGTLVSVSS

SEQ ID NO: 37 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFHLWGRGTLVSVSS

SEQ ID NO: 38 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDHWGRGTLVSVSS

SEQ ID NO: 39 - protein, 108 aa, synthetic construct
EIVLTQSPATLSLSPGERATLSCHASQSVSSYLAWYQQKPGQAPRLLIYDASKRATGIPARFSGSGSGTDFTLTISSLEPEDFAVYYCQQRSKWPPWTFGQGTKVESK

SEQ ID NO: 40 - protein, 108 aa, synthetic construct
EIVLTQSPATLSLSPGERATLSCRHSQSVSSYLAWYQQKPGQAPRLLIYDASKRATGIPARFSGSGSGTDFTLTISSLEPEDFAVYYCQQRSKWPPWTFGQGTKVESK

SEQ ID NO: 41 - protein, 108 aa, synthetic construct
EIVLTQSPATLSLSPGERATLSCRAHQSVSSYLAWYQQKPGQAPRLLIYDASKRATGIPARFSGSGSGTDFTLTISSLEPEDFAVYYCQQRSKWPPWTFGQGTKVESK

SEQ ID NO: 42 - protein, 108 aa, synthetic construct
EIVLTQSPATLSLSPGERATLSCRASHSVSSYLAWYQQKPGQAPRLLIYDASKRATGIPARFSGSGSGTDFTLTISSLEPEDFAVYYCQQRSKWPPWTFGQGTKVESK

SEQ ID NO: 43 - protein, 108 aa, synthetic construct
EIVLTQSPATLSLSPGERATLSCRASQHVSSYLAWYQQKPGQAPRLLIYDASKRATGIPARFSGSGSGTDFTLTISSLEPEDFAVYYCQQRSKWPPWTFGQGTKVESK

SEQ ID NO: 44 - protein, 108 aa, synthetic construct
EIVLTQSPATLSLSPGERATLSCRASQSHSSYLAWYQQKPGQAPRLLIYDASKRATGIPARFSGSGSGTDFTLTISSLEPEDFAVYYCQQRSKWPPWTFGQGTKVESK

SEQ ID NO: 45 - protein, 108 aa, synthetic construct
EIVLTQSPATLSLSPGERATLSCRASQSVHSYLAWYQQKPGQAPRLLIYDASKRATGIPARFSGSGSGTDFTLTISSLEPEDFAVYYCQQRSKWPPWTFGQGTKVESK

SEQ ID NO: 46 - protein, 108 aa, synthetic construct
EIVLTQSPATLSLSPGERATLSCRASQSVSHYLAWYQQKPGQAPRLLIYDASKRATGIPARFSGSGSGTDFTLTISSLEPEDFAVYYCQQRSKWPPWTFGQGTKVESK

SEQ ID NO: 47 - protein, 108 aa, synthetic construct
EIVLTQSPATLSLSPGERATLSCRASQSVSSHLAWYQQKPGQAPRLLIYDASKRATGIPARFSGSGSGTDFTLTISSLEPEDFAVYYCQQRSKWPPWTFGQGTKVESK

SEQ ID NO: 48 - protein, 108 aa, synthetic construct
EIVLTQSPATLSLSPGERATLSCRASQSVSSYHAWYQQKPGQAPRLLIYDASKRATGIPARFSGSGSGTDFTLTISSLEPEDFAVYYCQQRSKWPPWTFGQGTKVESK

SEQ ID NO: 49 - protein, 108 aa, synthetic construct
EIVLTQSPATLSLSPGERATLSCRASQSVSSYLHWYQQKPGQAPRLLIYDASKRATGIPARFSGSGSGTDFTLTISSLEPEDFAVYYCQQRSKWPPWTFGQGTKVESK

SEQ ID NO: 50 - protein, 108 aa, synthetic construct
EIVLTQSPATLSLSPGERATLSCRASQSVSSYLAWYQQKPGQAPRLLIYHASKRATGIPARFSGSGSGTDFTLTISSLEPEDFAVYYCQQRSKWPPWTFGQGTKVESK

SEQ ID NO: 51 - protein, 108 aa, synthetic construct
EIVLTQSPATLSLSPGERATLSCRASQSVSSYLAWYQQKPGQAPRLLIYDHSKRATGIPARFSGSGSGTDFTLTISSLEPEDFAVYYCQQRSKWPPWTFGQGTKVESK

SEQ ID NO: 52 - protein, 108 aa, synthetic construct
EIVLTQSPATLSLSPGERATLSCRASQSVSSYLAWYQQKPGQAPRLLIYDAHKRATGIPARFSGSGSGTDFTLTISSLEPEDFAVYYCQQRSKWPPWTFGQGTKVESK

SEQ ID NO: 53 - protein, 108 aa, synthetic construct
EIVLTQSPATLSLSPGERATLSCRASQSVSSYLAWYQQKPGQAPRLLIYDASHRATGIPARFSGSGSGTDFTLTISSLEPEDFAVYYCQQRSKWPPWTFGQGTKVESK

SEQ ID NO: 54 - protein, 108 aa, synthetic construct
EIVLTQSPATLSLSPGERATLSCRASQSVSSYLAWYQQKPGQAPRLLIYDASKHATGIPARFSGSGSGTDFTLTISSLEPEDFAVYYCQQRSKWPPWTFGQGTKVESK

SEQ ID NO: 55 - protein, 108 aa, synthetic construct
EIVLTQSPATLSLSPGERATLSCRASQSVSSYLAWYQQKPGQAPRLLIYDASKRHTGIPARFSGSGSGTDFTLTISSLEPEDFAVYYCQQRSKWPPWTFGQGTKVESK

SEQ ID NO: 56 - protein, 108 aa, synthetic construct
EIVLTQSPATLSLSPGERATLSCRASQSVSSYLAWYQQKPGQAPRLLIYDASKRAHGIPARFSGSGSGTDFTLTISSLEPEDFAVYYCQQRSKWPPWTFGQGTKVESK

SEQ ID NO: 57 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCHQRSKWPPWTFGQGTKVESK

SEQ ID NO: 58 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQHRSKWPPWTFGQGTKVESK

SEQ ID NO: 59 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQHSKWPPWTFGQGTKVESK

SEQ ID NO: 60 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRHKWPPWTFGQGTKVESK

SEQ ID NO: 61 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSHWPPWTFGQGTKVESK

SEQ ID NO: 62 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKHPPWTFGQGTKVESK

SEQ ID NO: 63 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWHPWTFGQGTKVESK

SEQ ID NO: 64 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPHWTFGQGTKVESK

SEQ ID NO: 65 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPHTFGQGTKVESK

SEQ ID NO: 66 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWHFGQGTKVESK

SEQ ID NO: 67 - protein, 118 aa, synthetic construct
QVQLVESGGGVVQPGRSLRLSCAASGFTFSDYAMHWVRQAPGKGLEWVAIIWFDGSSTYYADSVRGRFTISRDNSKNTLYLQMNSLRAEDTAVYYCARELGRRYFDLWGRGTLVTVSS

SEQ ID NO: 68 - protein, 118 aa, synthetic construct
QVQLVESGGGVVQPGRSLRLSCAASGFTFSDYYMSWVRQAPGKGLEWVAIIWFDGSSTYYADSVRGRFTISRDNSKNTLYLQMNSLRAEDTAVYYCARELGRRYFDLWGRGTLVTVSS

SEQ ID NO: 69 - protein, 118 aa, synthetic construct
QVQLVESGGGVVQPGRSLRLSCAASGFTFSDYGMSWVRQAPGKGLEWVAIIWFDGSSTYYADSVRGRFTISRDNSKNTLYLQMNSLRAEDTAVYYCARELGRRYFDLWGRGTLVTVSS

SEQ ID NO: 70 - protein, 118 aa, synthetic construct
QVQLVESGGGVVQPGRSLRLSCAASGFTFSDYTMHWVRQAPGKGLEWVAIIWFDGSSTYYADSVRGRFTISRDNSKNTLYLQMNSLRAEDTAVYYCARELGRRYFDLWGRGTLVTVSS

SEQ ID NO: 71 - protein, 118 aa, synthetic construct
QVQLVESGGGVVQPGRSLRLSCAASGFTFSDHYMDWVRQAPGKGLEWVAIIWFDGSSTYYADSVRGRFTISRDNSKNTLYLQMNSLRAEDTAVYYCARELGRRYFDLWGRGTLVTVSS

SEQ ID NO: 72 - protein, 118 aa, synthetic construct
QVQLVESGGGVVQPGRSLRLSCAASGFTFSSYDMHWVRQAPGKGLEWVAIIWFDGSSTYYADSVRGRFTISRDNSKNTLYLQMNSLRAEDTAVYYCARELGRRYFDLWGRGTLVTVSS

SEQ ID NO: 73 - protein, 118 aa, synthetic construct
QVQLVESGGGVVQPGRSLRLSCAASGFTFSSYSMHWVRQAPGKGLEWVAIIWFDGSSTYYADSVRGRFTISRDNSKNTLYLQMNSLRAEDTAVYYCARELGRRYFDLWGRGTLVTVSS

SEQ ID NO: 74 - protein, 118 aa, synthetic construct
QVQLVESGGGVVQPGRSLRLSCAASGFTFSSYSMNWVRQAPGKGLEWVAIIWFDGSSTYYADSVRGRFTISRDNSKNTLYLQMNSLRAEDTAVYYCARELGRRYFDLWGRGTLVTVSS

SEQ ID NO: 75 - protein, 118 aa, synthetic construct
QVQLVESGGGVVQPGRSLRLSCAASGFTFSDYAMSWVRQAPGKGLEWVAIIWFDGSSTYYADSVRGRFTISRDNSKNTLYLQMNSLRAEDTAVYYCARELGRRYFDLWGRGTLVTVSS

SEQ ID NO: 76 - protein, 118 aa, synthetic construct
QVQLVESGGGVVQPGRSLRLSCAASGFTFSSYGMHWVRQAPGKGLEWVAIIWFDGSSHYYADSVKGRFTISRDNSKNTLYLQMNSLRAEDTAVYYCARELGRRYFDLWGRGTLVTVSS

SEQ ID NO: 77 - protein, 118 aa, synthetic construct
QVQLVESGGGVVQPGRSLRLSCAASGFTFSSYGMHWVRQAPGKGLEWVAIIWFDGSSKYYADSVKGRFTISRDNSKNTLYLQMNSLRAEDTAVYYCARELGRRYFDLWGRGTLVTVSS

SEQ ID NO: 78 - protein, 118 aa, synthetic construct
QVQLVESGGGVVQPGRSLRLSCAASGFTFSSYGMHWVRQAPGKGLEWVAIIWFDGSSHYYVDSVKGRFTISRDNSKNTLYLQMNSLRAEDTAVYYCARELGRRYFDLWGRGTLVTVSS

SEQ ID NO: 79 - protein, 118 aa, synthetic construct
QVQLVESGGGVVQPGRSLRLSCAASGFTFSSYGMHWVRQAPGKGLEWVAIIWFDGSSKYYVDSVKGRFTISRDNSKNTLYLQMNSLRAEDTAVYYCARELGRRYFDLWGRGTLVTVSS

SEQ ID NO: 80 - protein, 118 aa, synthetic construct
QVQLVESGGGVVQPGRSLRLSCAASGFTFSSYGMHWVRQAPGKGLEWVAIIWFDGSSHYYPGSVKGRFTISRDNSKNTLYLQMNSLRAEDTAVYYCARELGRRYFDLWGRGTLVTVSS

SEQ ID NO: 81 - protein, 118 aa, synthetic construct
QVQLVESGGGVVQPGRSLRLSCAASGFTFSSYGMHWVRQAPGKGLEWVAIIWFDGSSKYYPGSVKGRFTISRDNSKNTLYLQMNSLRAEDTAVYYCARELGRRYFDLWGRGTLVTVSS

SEQ ID NO: 82 - protein, 118 aa, synthetic construct
QVQLVESGGGVVQPGRSLRLSCAASGFTFSSYGMHWVRQAPGKGLEWVAVIWYDGSSHYYADSVKGRFTISRDNSKNTLYLQMNSLRAEDTAVYYCARELGRRYFDLWGRGTLVTVSS

SEQ ID NO: 83 - protein, 118 aa, synthetic construct
QVQLVESGGGVVQPGRSLRLSCAASGFTFSSYGMHWVRQAPGKGLEWVAVIWYDGSSKYYADSVKGRFTISRDNSKNTLYLQMNSLRAEDTAVYYCARELGRRYFDLWGRGTLVTVSS

SEQ ID NO: 84 - protein, 118 aa, synthetic construct
QVQLVESGGGVVQPGRSLRLSCAASGFTFSSYGMHWVRQAPGKGLEWVAVIWYDGSSHYYVDSVKGRFTISRDNSKNTLYLQMNSLRAEDTAVYYCARELGRRYFDLWGRGTLVTVSS

SEQ ID NO: 85 - protein, 118 aa, synthetic construct
QVQLVESGGGVVQPGRSLRLSCAASGFTFSSYGMHWVRQAPGKGLEWVAVIWYDGSSKYYVDSVKGRFTISRDNSKNTLYLQMNSLRAEDTAVYYCARELGRRYFDLWGRGTLVTVSS

SEQ ID NO: 86 - protein, 118 aa, synthetic construct
QVQLVESGGGVVQPGRSLRLSCAASGFTFSSYGMHWVRQAPGKGLEWVAVIWYDGSSHYYPGSVKGRFTISRDNSKNTLYLQMNSLRAEDTAVYYCARELGRRYFDLWGRGTLVTVSS

SEQ ID NO: 87 - protein, 118 aa, synthetic construct
QVQLVESGGGVVQPGRSLRLSCAASGFTFSSYGMHWVRQAPGKGLEWVAVIWYDGSSKYYPGSVKGRFTISRDNSKNTLYLQMNSLRAEDTAVYYCARELGRRYFDLWGRGTLVTVSS

SEQ ID NO: 88 - protein, 118 aa, synthetic construct
QVQLVESGGGVVQPGRSLRLSCAASGFTFSSYGMHWVRQAPGKGLEWVAIIWFDGSSTYYADSVRGRFTISRDNSKNTLYLQMNSLRAEDTAVYYCARELGRRYFDIWGRGTLVTVSS

SEQ ID NO: 89 - protein, 108 aa, synthetic construct
EIVLTQSPATLSLSPGERATLSCRASQSVSSNLAWYQQKPGQAPRLLIYDASKRATGIPARFSGSGSGTDFTLTISSLEPEDFAVYYCQQRSKWPPWTFGQGTKVEIK

SEQ ID NO: 90 - protein, 109 aa, synthetic construct
EIVLTQSPATLSLSPGERATLSCRASQSVSSSYLAWYQQKPGQAPRLLIYDASKRATGIPARFSGSGSGTDFTLTISSLEPEDFAVYYCQQRSKWPPWTFGQGTKVEIK

SEQ ID NO: 91 - protein, 108 aa, synthetic construct
EIVLTQSPATLSLSPGERATLSCRASQGVSSYLAWYQQKPGQAPRLLIYDASKRATGIPARFSGSGSGTDFTLTISSLEPEDFAVYYCQQRSKWPPWTFGQGTKVEIK

SEQ ID NO: 92 - protein, 109 aa, synthetic construct
EIVLTQSPATLSLSPGERATLSCGASQSVSSSYLAWYQQKPGQAPRLLIYDASKRATGIPARFSGSGSGTDFTLTISSLEPEDFAVYYCQQRSKWPPWTFGQGTKVEIK

SEQ ID NO: 93 - protein, 109 aa, synthetic construct
EIVLTQSPATLSLSPGERATLSCRASQSVSSSYLSWYQQKPGQAPRLLIYDASKRATGIPARFSGSGSGTDFTLTISSLEPEDFAVYYCQQRSKWPPWTFGQGTKVEIK

SEQ ID NO: 94 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSKWPPWTFGQGTKVEIK

SEQ ID NO: 95 - protein, 108 aa, synthetic construct
EIVLTQSPATLSLSPGERATLSCRASQSVSSYLAWYQQKPGQAPRLLIYGASTRATGIPARFSGSGSGTDFTLTISSLEPEDFAVYYCQQRSKWPPWTFGQGTKVEIK

SEQ ID NO: 96 - protein, 108 aa, synthetic construct
EIVLTQSPATLSLSPGERATLSCRASQSVSSYLAWYQQKPGQAPRLLIYGASSRATGIPARFSGSGSGTDFTLTISSLEPEDFAVYYCQQRSKWPPWTFGQGTKVEIK

SEQ ID NO: 97 - protein, 108 aa, synthetic construct
EIVLTQSPATLSLSPGERATLSCRASQSVSSYLAWYQQKPGQAPRLLIYDASSRATGIPARFSGSGSGTDFTLTISSLEPEDFAVYYCQQRSKWPPWTFGQGTKVEIK

SEQ ID NO: 98 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSRWPPWTFGQGTKVEIK

SEQ ID NO: 99 - protein, 118 aa, synthetic construct
QVQLVESGGGVVQPGRSLRLSCAASGFTFSSYSMHWVRQAPGKGLEWVAIIWFDGSSKYYADSVKGRFTISRDNSKNTLYLQMNSLRAEDTAVYYCARELGRRYFDIWGRGTLVTVSS

SEQ ID NO: 100 - protein, 108 aa, synthetic construct
EIVLTQSPATLSLSPGERATLSCRASQGVSSYLAWYQQKPGQAPRLLIYGASTRATGIPARFSGSGSGTDFTLTISSLEPEDFAVYYCQQRSRWPPWTFGQGTKLEIK

SEQ ID NO: 101 - protein, 118 aa, synthetic construct
QVQLVESGGGVVQPGRSLRLSCAASGFTFSSYSMHWVRQAPGKGLEWVAIIWFDGSSKYYVDSVKGRFTISRDNSKNTLYLQMNSLRAEDTAVYYCARELGRRYFDIWGRGTLVTVSS

SEQ ID NO: 102 - protein, 118 aa, synthetic construct
QVQLVESGGGVVQPGRSLRLSCAASGFTFSSYSMHWVRQAPGKGLEWVAIIWFDGSSKYYPGSVKGRFTISRDNSKNTLYLQMNSLRAEDTAVYYCARELGRRYFDIWGRGTLVTVSS

SEQ ID NO: 103 - protein, 118 aa, synthetic construct
QVQLVESGGGVVQPGRSLRLSCAASGFTFSDYTMHWVRQAPGKGLEWVAIIWFDGSSKYYADSVKGRFTISRDNSKNTLYLQMNSLRAEDTAVYYCARELGRRYFDIWGRGTLVTVSS

SEQ ID NO: 104 - protein, 118 aa, synthetic construct
QVQLVESGGGVVQPGRSLRLSCAASGFTFSDYTMHWVRQAPGKGLEWVAIIWFDGSSKYYVDSVKGRFTISRDNSKNTLYLQMNSLRAEDTAVYYCARELGRRYFDIWGRGTLVTVSS

SEQ ID NO: 105 - protein, 118 aa, synthetic construct
QVQLVESGGGVVQPGRSLRLSCAASGFTFSDYTMHWVRQAPGKGLEWVAIIWFDGSSKYYPGSVKGRFTISRDNSKNTLYLQMNSLRAEDTAVYYCARELGRRYFDIWGRGTLVTVSS

SEQ ID NO: 106 - protein, 118 aa, synthetic construct
QVQLVESGGGVVQPGRSLRLSCAASGFTFSDHYMDWVRQAPGKGLEWVAIIWFDGSSKYYADSVKGRFTISRDNSKNTLYLQMNSLRAEDTAVYYCARELGRRYFDIWGRGTLVTVSS

SEQ ID NO: 107 - protein, 118 aa, synthetic construct
QVQLVESGGGVVQPGRSLRLSCAASGFTFSDHYMDWVRQAPGKGLEWVAIIWFDGSSKYYVDSVKGRFTISRDNSKNTLYLQMNSLRAEDTAVYYCARELGRRYFDIWGRGTLVTVSS

SEQ ID NO: 108 - protein, 118 aa, synthetic construct
QVQLVESGGGVVQPGRSLRLSCAASGFTFSDHYMDWVRQAPGKGLEWVAIIWFDGSSKYYPGSVKGRFTISRDNSKNTLYLQMNSLRAEDTAVYYCARELGRRYFDIWGRGTLVTVSS

SEQ ID NO: 109 - protein, 108 aa, synthetic construct
EIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 110 - protein, 118 aa, synthetic construct
EVQLVESGGGVVQPGRSLRLSCAASGFTFSDYTMHWVRQAPGKGLEWVAIIWFDGSSKYYPGSVKGRFTISRDNSKNTLYLQMNSLRAEDTAVYYCARELGRRYFDIWGRGTLVTVSS

SEQ ID NO: 111 - protein, 118 aa, synthetic construct
EVQLVESGGGVVQPGRSLRLSCAASGFTFSSYSMHWVRQAPGKGLEWVAIIWFDGSNKYYADSVKSRFTISRDNSKNTLYLQMNSLRAEDTAVYYCARELGRRYFDIWGQGTLVTVSS

SEQ ID NO: 112 - protein, 118 aa, synthetic construct
EVQLVESGGGVVQPGRSLRLSCAASGFTFSSYSMHWVRQAPGKGLEWVAIIWFDGSSKYYVDSVKSRFTISRDNSKNTLYLQMNSLRAEDTAVYYCARELGRRYFDIWGQGTLVTVSS

SEQ ID NO: 113 - protein, 118 aa, synthetic construct
EVQLVESGGGVVQPGRSLRLSCAASGFTFSSYSMHWVRQAPGKGLEWVAIIWFDGSEKYYVDSVKSRFTISRDNSKNTLYLQMNSLRAEDTAVYYCARELGRRYFDIWGQGTLVTVSS

SEQ ID NO: 114 - protein, 118 aa, synthetic construct
EVQLVESGGGVVQPGRSLRLSCAASGFTFSSYSMHWVRQAPGKGLEWVAVIWYDGSSKYYADSVKGRFTISRDNSKNTLYLQMNSLRAEDTAVYYCARELGRRYFDIWGQGTLVTVSS

SEQ ID NO: 115 - protein, 118 aa, synthetic construct
EVQLVESGGGVVQPGRSLRLSCAASGFTFSSYSMHWVRQAPGKGLEWVAVIWYDGSSKYYADSVKSRFTISRDNSKNTLYLQMNSLRAEDTAVYYCARELGRRYFDIWGRGTLVTVSS

SEQ ID NO: 116 - protein, 118 aa, synthetic construct
EVQLVESGGGVVQPGRSLRLSCAASGFTFSSYSMHWVRQAPGKGLEWVAVIWYDGSSKYYADSVKSRFTISRDNSKNTLYLQMNSLRAEDTAVYYCARELGRRYFDIWGQGTLVTVSS

SEQ ID NO: 117 - protein, 118 aa, synthetic construct
EVQLVESGGGVVQPGRSLRLSCAASGFTFDDYAMHWVRQAPGKGLEWVAIIWFDGSSKYYADSVKGRFTISRDNSKNTLYLQMNSLRAEDTAVYYCARELGRRYFDIWGQGTLVTVSS

SEQ ID NO: 118 - protein, 118 aa, synthetic construct
EVQLVESGGGVVQPGRSLRLSCAASGFTFDDYTMHWVRQAPGKGLEWVAIIWFDGSSKYYADSVKGRFTISRDNSKNTLYLQMNSLRAEDTAVYYCARELGRRYFDIWGQGTLVTVSS

SEQ ID NO: 119 - protein, 118 aa, synthetic construct
EVQLVESGGGVVQPGRSLRLSCAASGFTFDDYAMHWVRQAPGKGLEWVAVIWYDGSSKYYADSVKGRFTISRDNSKNTLYLQMNSLRAEDTAVYYCARELGRRYFDIWGQGTLVTVSS

SEQ ID NO: 120 - protein, 118 aa, synthetic construct
EVQLVESGGGVVQPGRSLRLSCAASGFTFDDYTMHWVRQAPGKGLEWVAVIWYDGSSKYYADSVKGRFTISRDNSKNTLYLQMNSLRAEDTAVYYCARELGRRYFDIWGQGTLVTVSS

SEQ ID NO: 121 - protein, 118 aa, synthetic construct
EVQLVESGGGVVQPGRSLRLSCAASGFTFSDYTMHWVRQAPGKGLEWVAIIWFDGSSKYYADSVKSRFTISRDNSKNTLYLQMNSLRAEDTAVYYCARELGRRYFDIWGQGTLVTVSS

SEQ ID NO: 122 - protein, 108 aa, synthetic construct
EIVMTQSPATLSVSPGERATLSCRASQGVSSNLAWYQQKPGQAPRLLIYGASTRATGIPARFSGSGSGTEFTLTISSLQSEDFAVYYCQQRSRWPPWTFGGGTKLEIK

SEQ ID NO: 123 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 124 - protein, 226 aa, synthetic construct
QVQLVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 125 - protein, 226 aa, synthetic construct
QVELVESGGGVVQPGRSLRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 126 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYYCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 127 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 128 - protein, 226 aa, synthetic construct
QVQLVESGGGVVQPGRSLRLSCAASGFTFSSYGMHWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 129 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 130 - protein, 226 aa, synthetic construct
QVQLVESGGGVVQPGRSLRLSCAASGFTFSSYGMHWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 131 - protein, 226 aa, synthetic construct
QVELVESGGGVVQPGRSQRLSCAASGFTFSHYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 132 - protein, 226 aa, synthetic construct
QVELVESGGGVVQPGRSQRLSCAASGFTFSSHGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 133 - protein, 226 aa, synthetic construct
QVELVESGGGVVQPGRSQRLSCAASGFTFSSYHMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 134 - protein, 226 aa, synthetic construct
QVELVESGGGVVQPGRSQRLSCAASGFTFSSYGHHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 135 - protein, 226 aa, synthetic construct
QVELVESGGGVVQPGRSQRLSCAASGFTFSSYGMHWVRQAPGKGLEWVAHIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 136 - protein, 226 aa, synthetic construct
QVELVESGGGVVQPGRSQRLSCAASGFTFSSYGMHWVRQAPGKGLEWVAIHWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 137 - protein, 226 aa, synthetic construct
QVELVESGGGVVQPGRSQRLSCAASGFTFSSYGMHWVRQAPGKGLEWVAIIHFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 138 - protein, 226 aa, synthetic construct
QVELVESGGGVVQPGRSQRLSCAASGFTFSSYGMHWVRQAPGKGLEWVAIIWHDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 139 - protein, 226 aa, synthetic construct
QVELVESGGGVVQPGRSQRLSCAASGFTFSSYGMHWVRQAPGKGLEWVAIIWFHGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 140 - protein, 226 aa, synthetic construct
QVELVESGGGVVQPGRSQRLSCAASGFTFSSYGMHWVRQAPGKGLEWVAIIWFDHSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 141 - protein, 226 aa, synthetic construct
QVELVESGGGVVQPGRSQRLSCAASGFTFSSYGMHWVRQAPGKGLEWVAIIWFDGHSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 142 - protein, 226 aa, synthetic construct
QVELVESGGGVVQPGRSQRLSCAASGFTFSSYGMHWVRQAPGKGLEWVAIIWFDGSHTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 143 - protein, 226 aa, synthetic construct
QVELVESGGGVVQPGRSQRLSCAASGFTFSSYGMHWVRQAPGKGLEWVAIIWFDGSSHYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 144 - protein, 226 aa, synthetic construct
QVELVESGGGVVQPGRSQRLSCAASGFTFSSYGMHWVRQAPGKGLEWVAIIWFDGSSTHYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 145 - protein, 226 aa, synthetic construct
QVELVESGGGVVQPGRSQRLSCAASGFTFSSYGMHWVRQAPGKGLEWVAIIWFDGSSTYHADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 146 - protein, 226 aa, synthetic construct
QVELVESGGGVVQPGRSQRLSCAASGFTFSSYGMHWVRQAPGKGLEWVAIIWFDGSSTYYHDSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 147 - protein, 226 aa, synthetic construct
QVELVESGGGVVQPGRSQRLSCAASGFTFSSYGMHWVRQAPGKGLEWVAIIWFDGSSTYYAHSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 148 - protein, 226 aa, synthetic construct
QVELVESGGGVVQPGRSQRLSCAASGFTFSSYGMHWVRQAPGKGLEWVAIIWFDGSSTYYADHVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 149 - protein, 226 aa, synthetic construct
QVELVESGGGVVQPGRSQRLSCAASGFTFSSYGMHWVRQAPGKGLEWVAIIWFDGSSTYYADSHRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 150 - protein, 226 aa, synthetic construct
QVELVESGGGVVQPGRSQRLSCAASGFTFSSYGMHWVRQAPGKGLEWVAIIWFDGSSTYYADSVHGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 151 - protein, 226 aa, synthetic construct
QVELVESGGGVVQPGRSQRLSCAASGFTFSSYGMHWVRQAPGKGLEWVAIIWFDGSSTYYADSVRHRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 152 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARHLGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 153 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCAREHGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 154 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELHRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 155 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGHRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 156 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRHYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 157 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRHFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 158 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYHDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 159 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFHLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 160 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDHWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 161 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCHASQSVSSYLAWYQQKPGQAPRLLIYDASKRATGIPARFSGSGSGTDFTLTISSLEPEDFAVYYCQQRSKWPPWTFGQGTKVESK

SEQ ID NO: 162 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRHSQSVSSYLAWYQQKPGQAPRLLIYDASKRATGIPARFSGSGSGTDFTLTISSLEPEDFAVYYCQQRSKWPPWTFGQGTKVESK

SEQ ID NO: 163 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRAHQSVSSYLAWYQQKPGQAPRLLIYDASKRATGIPARFSGSGSGTDFTLTISSLEPEDFAVYYCQQRSKWPPWTFGQGTKVESK

SEQ ID NO: 164 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASHSVSSYLAWYQQKPGQAPRLLIYDASKRATGIPARFSGSGSGTDFTLTISSLEPEDFAVYYCQQRSKWPPWTFGQGTKVESK

SEQ ID NO: 165 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQHVSSYLAWYQQKPGQAPRLLIYDASKRATGIPARFSGSGSGTDFTLTISSLEPEDFAVYYCQQRSKWPPWTFGQGTKVESK

SEQ ID NO: 166 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSHSSYLAWYQQKPGQAPRLLIYDASKRATGIPARFSGSGSGTDFTLTISSLEPEDFAVYYCQQRSKWPPWTFGQGTKVESK

SEQ ID NO: 167 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVHSYLAWYQQKPGQAPRLLIYDASKRATGIPARFSGSGSGTDFTLTISSLEPEDFAVYYCQQRSKWPPWTFGQGTKVESK

SEQ ID NO: 168 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSHYLAWYQQKPGQAPRLLIYDASKRATGIPARFSGSGSGTDFTLTISSLEPEDFAVYYCQQRSKWPPWTFGQGTKVESK

SEQ ID NO: 169 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSHLAWYQQKPGQAPRLLIYDASKRATGIPARFSGSGSGTDFTLTISSLEPEDFAVYYCQQRSKWPPWTFGQGTKVESK

SEQ ID NO: 170 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYHAWYQQKPGQAPRLLIYDASKRATGIPARFSGSGSGTDFTLTISSLEPEDFAVYYCQQRSKWPPWTFGQGTKVESK

SEQ ID NO: 171 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLHWYQQKPGQAPRLLIYDASKRATGIPARFSGSGSGTDFTLTISSLEPEDFAVYYCQQRSKWPPWTFGQGTKVESK

SEQ ID NO: 172 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYHASKRATGIPARFSGSGSGTDFTLTISSLEPEDFAVYYCQQRSKWPPWTFGQGTKVESK

SEQ ID NO: 173 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDHSKRATGIPARFSGSGSGTDFTLTISSLEPEDFAVYYCQQRSKWPPWTFGQGTKVESK

SEQ ID NO: 174 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDAHKRATGIPARFSGSGSGTDFTLTISSLEPEDFAVYYCQQRSKWPPWTFGQGTKVESK

SEQ ID NO: 175 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASHRATGIPARFSGSGSGTDFTLTISSLEPEDFAVYYCQQRSKWPPWTFGQGTKVESK

SEQ ID NO: 176 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKHATGIPARFSGSGSGTDFTLTISSLEPEDFAVYYCQQRSKWPPWTFGQGTKVESK

SEQ ID NO: 177 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRHTGIPARFSGSGSGTDFTLTISSLEPEDFAVYYCQQRSKWPPWTFGQGTKVESK

SEQ ID NO: 178 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRAHGIPARFSGSGSGTDFTLTISSLEPEDFAVYYCQQRSKWPPWTFGQGTKVESK

SEQ ID NO: 179 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCHQRSKWPPWTFGQGTKVESK

SEQ ID NO: 180 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQHRSKWPPWTFGQGTKVESK

SEQ ID NO: 181 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQHSKWPPWTFGQGTKVESK

SEQ ID NO: 182 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRHKWPPWTFGQGTKVESK

SEQ ID NO: 183 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSHWPPWTFGQGTKVESK

SEQ ID NO: 184 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKHPPWTFGQGTKVESK

SEQ ID NO: 185 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWHPWTFGQGTKVESK

SEQ ID NO: 186 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPHWTFGQGTKVESK

SEQ ID NO: 187 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPHTFGQGTKVESK

SEQ ID NO: 188 - protein, 226 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWHFGQGTKVESK

SEQ ID NO: 189 - protein, 226 aa, synthetic construct
QVQLVESGGGVVQPGRSLRLSCAASGFTFSDYAMHWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 190 - protein, 226 aa, synthetic construct
QVQLVESGGGVVQPGRSLRLSCAASGFTFSDYYMSWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 191 - protein, 226 aa, synthetic construct
QVQLVESGGGVVQPGRSLRLSCAASGFTFSDYGMSWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 192 - protein, 226 aa, synthetic construct
QVQLVESGGGVVQPGRSLRLSCAASGFTFSDYTMHWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 193 - protein, 226 aa, synthetic construct
QVQLVESGGGVVQPGRSLRLSCAASGFTFSDHYMDWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 194 - protein, 226 aa, synthetic construct
QVQLVESGGGVVQPGRSLRLSCAASGFTFSSYDMHWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 195 - protein, 226 aa, synthetic construct
QVQLVESGGGVVQPGRSLRLSCAASGFTFSSYSMHWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 196 - protein, 226 aa, synthetic construct
QVQLVESGGGVVQPGRSLRLSCAASGFTFSSYSMNWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 197 - protein, 226 aa, synthetic construct
QVQLVESGGGVVQPGRSLRLSCAASGFTFSDYAMSWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 198 - protein, 226 aa, synthetic construct
QVQLVESGGGVVQPGRSLRLSCAASGFTFSSYGMHWVRQAPGKGLEWVAIIWFDGSSHYYADSVKGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 199 - protein, 226 aa, synthetic construct
QVQLVESGGGVVQPGRSLRLSCAASGFTFSSYGMHWVRQAPGKGLEWVAIIWFDGSSKYYADSVKGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 200 - protein, 226 aa, synthetic construct
QVQLVESGGGVVQPGRSLRLSCAASGFTFSSYGMHWVRQAPGKGLEWVAIIWFDGSSHYYVDSVKGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 201 - protein, 226 aa, synthetic construct
QVQLVESGGGVVQPGRSLRLSCAASGFTFSSYGMHWVRQAPGKGLEWVAIIWFDGSSKYYVDSVKGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 202 - protein, 226 aa, synthetic construct
QVQLVESGGGVVQPGRSLRLSCAASGFTFSSYGMHWVRQAPGKGLEWVAIIWFDGSSHYYPGSVKGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 203 - protein, 226 aa, synthetic construct
QVQLVESGGGVVQPGRSLRLSCAASGFTFSSYGMHWVRQAPGKGLEWVAIIWFDGSSKYYPGSVKGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 204 - protein, 226 aa, synthetic construct
QVQLVESGGGVVQPGRSLRLSCAASGFTFSSYGMHWVRQAPGKGLEWVAVIWYDGSSHYYADSVKGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 205 - protein, 226 aa, synthetic construct
QVQLVESGGGVVQPGRSLRLSCAASGFTFSSYGMHWVRQAPGKGLEWVAVIWYDGSSKYYADSVKGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 206 - protein, 226 aa, synthetic construct
QVQLVESGGGVVQPGRSLRLSCAASGFTFSSYGMHWVRQAPGKGLEWVAVIWYDGSSHYYVDSVKGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 207 - protein, 226 aa, synthetic construct
QVQLVESGGGVVQPGRSLRLSCAASGFTFSSYGMHWVRQAPGKGLEWVAVIWYDGSSKYYVDSVKGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 208 - protein, 226 aa, synthetic construct
QVQLVESGGGVVQPGRSLRLSCAASGFTFSSYGMHWVRQAPGKGLEWVAVIWYDGSSHYYPGSVKGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 209 - protein, 226 aa, synthetic construct
QVQLVESGGGVVQPGRSLRLSCAASGFTFSSYGMHWVRQAPGKGLEWVAVIWYDGSSKYYPGSVKGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 210 - protein, 226 aa, synthetic construct
QVQLVESGGGVVQPGRSLRLSCAASGFTFSSYGMHWVRQAPGKGLEWVAIIWFDGSSTYYADSVRGRFTISRDNSKNTLYLQMNSLRAEDTAVYYCARELGRRYFDIWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVEIK

SEQ ID NO: 211 - protein, 226 aa, synthetic construct
QVQLVESGGGVVQPGRSLRLSCAASGFTFSSYGMHWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSNLAWYQQKPGQAPRLLIYDASKRATGIPARFSGSGSGTDFTLTISSLEPEDFAVYYCQQRSKWPPWTFGQGTKVEIK

SEQ ID NO: 212 - protein, 227 aa, synthetic construct
QVQLVESGGGVVQPGRSLRLSCAASGFTFSSYGMHWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSSYLAWYQQKPGQAPRLLIYDASKRATGIPARFSGSGSGTDFTLTISSLEPEDFAVYYCQQRSKWPPWTFGQGTKVEIK

SEQ ID NO: 213 - protein, 226 aa, synthetic construct
QVQLVESGGGVVQPGRSLRLSCAASGFTFSSYGMHWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQGVSSYLAWYQQKPGQAPRLLIYDASKRATGIPARFSGSGSGTDFTLTISSLEPEDFAVYYCQQRSKWPPWTFGQGTKVEIK

SEQ ID NO: 214 - protein, 227 aa, synthetic construct
QVQLVESGGGVVQPGRSLRLSCAASGFTFSSYGMHWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCGASQSVSSSYLAWYQQKPGQAPRLLIYDASKRATGIPARFSGSGSGTDFTLTISSLEPEDFAVYYCQQRSKWPPWTFGQGTKVEIK

SEQ ID NO: 215 - protein, 227 aa, synthetic construct
QVQLVESGGGVVQPGRSLRLSCAASGFTFSSYGMHWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSSYLSWYQQKPGQAPRLLIYDASKRATGIPARFSGSGSGTDFTLTISSLEPEDFAVYYCQQRSKWPPWTFGQGTKVEIK

SEQ ID NO: 216 - protein, 226 aa, synthetic construct
QVQLVESGGGVVQPGRSLRLSCAASGFTFSSYGMHWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASNRATGIPARFSGSGSGTDFTLTISSLEPEDFAVYYCQQRSKWPPWTFGQGTKVEIK

SEQ ID NO: 217 - protein, 226 aa, synthetic construct
QVQLVESGGGVVQPGRSLRLSCAASGFTFSSYGMHWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYGASTRATGIPARFSGSGSGTDFTLTISSLEPEDFAVYYCQQRSKWPPWTFGQGTKVEIK

SEQ ID NO: 218 - protein, 226 aa, synthetic construct
QVQLVESGGGVVQPGRSLRLSCAASGFTFSSYGMHWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYGASSRATGIPARFSGSGSGTDFTLTISSLEPEDFAVYYCQQRSKWPPWTFGQGTKVEIK

SEQ ID NO: 219 - protein, 226 aa, synthetic construct
QVQLVESGGGVVQPGRSLRLSCAASGFTFSSYGMHWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASSRATGIPARFSGSGSGTDFTLTISSLEPEDFAVYYCQQRSKWPPWTFGQGTKVEIK

SEQ ID NO: 220 - protein, 226 aa, synthetic construct
QVQLVESGGGVVQPGRSLRLSCAASGFTFSSYGMHWVRQAPGKGLEWVAIIWFDGSSTYYADSVRGRFTISRDNSKNTLYLQMNSLRAEDTAVYYCARELGRRYFDLWGRGTLVTVSSEIVLTQSPATLSLSPGERATLSCRASQSVSSYLAWYQQKPGQAPRLLIYDASKRATGIPARFSGSGSGTDFTLTISSLEPEDFAVYYCQQRSRWPPWTFGQGTKVEIK

SEQ ID NO: 221 - protein, 226 aa, synthetic construct
QVQLVESGGGVVQPGRSLRLSCAASGFTFSSYSMHWVRQAPGKGLEWVAIIWFDGSSKYYADSVKGRFTISRDNSKNTLYLQMNSLRAEDTAVYYCARELGRRYFDIWGRGTLVTVSSEIVLTQSPATLSLSPGERATLSCRASQGVSSYLAWYQQKPGQAPRLLIYGASTRATGIPARFSGSGSGTDFTLTISSLEPEDFAVYYCQQRSRWPPWTFGQGTKLEIK

SEQ ID NO: 222 - protein, 226 aa, synthetic construct
QVQLVESGGGVVQPGRSLRLSCAASGFTFSSYSMHWVRQAPGKGLEWVAIIWFDGSSKYYVDSVKGRFTISRDNSKNTLYLQMNSLRAEDTAVYYCARELGRRYFDIWGRGTLVTVSSEIVLTQSPATLSLSPGERATLSCRASQGVSSYLAWYQQKPGQAPRLLIYGASTRATGIPARFSGSGSGTDFTLTISSLEPEDFAVYYCQQRSRWPPWTFGQGTKLEIK

SEQ ID NO: 223 - protein, 226 aa, synthetic construct
QVQLVESGGGVVQPGRSLRLSCAASGFTFSSYSMHWVRQAPGKGLEWVAIIWFDGSSKYYPGSVKGRFTISRDNSKNTLYLQMNSLRAEDTAVYYCARELGRRYFDIWGRGTLVTVSSEIVLTQSPATLSLSPGERATLSCRASQGVSSYLAWYQQKPGQAPRLLIYGASTRATGIPARFSGSGSGTDFTLTISSLEPEDFAVYYCQQRSRWPPWTFGQGTKLEIK

SEQ ID NO: 224 - protein, 226 aa, synthetic construct
QVQLVESGGGVVQPGRSLRLSCAASGFTFSDYTMHWVRQAPGKGLEWVAIIWFDGSSKYYADSVKGRFTISRDNSKNTLYLQMNSLRAEDTAVYYCARELGRRYFDIWGRGTLVTVSSEIVLTQSPATLSLSPGERATLSCRASQGVSSYLAWYQQKPGQAPRLLIYGASTRATGIPARFSGSGSGTDFTLTISSLEPEDFAVYYCQQRSRWPPWTFGQGTKLEIK

SEQ ID NO: 225 - protein, 226 aa, synthetic construct
QVQLVESGGGVVQPGRSLRLSCAASGFTFSDYTMHWVRQAPGKGLEWVAIIWFDGSSKYYVDSVKGRFTISRDNSKNTLYLQMNSLRAEDTAVYYCARELGRRYFDIWGRGTLVTVSSEIVLTQSPATLSLSPGERATLSCRASQGVSSYLAWYQQKPGQAPRLLIYGASTRATGIPARFSGSGSGTDFTLTISSLEPEDFAVYYCQQRSRWPPWTFGQGTKLEIK

SEQ ID NO: 226 - protein, 226 aa, synthetic construct
QVQLVESGGGVVQPGRSLRLSCAASGFTFSDYTMHWVRQAPGKGLEWVAIIWFDGSSKYYPGSVKGRFTISRDNSKNTLYLQMNSLRAEDTAVYYCARELGRRYFDIWGRGTLVTVSSEIVLTQSPATLSLSPGERATLSCRASQGVSSYLAWYQQKPGQAPRLLIYGASTRATGIPARFSGSGSGTDFTLTISSLEPEDFAVYYCQQRSRWPPWTFGQGTKLEIK

SEQ ID NO: 227 - protein, 226 aa, synthetic construct
QVQLVESGGGVVQPGRSLRLSCAASGFTFSDHYMDWVRQAPGKGLEWVAIIWFDGSSKYYADSVKGRFTISRDNSKNTLYLQMNSLRAEDTAVYYCARELGRRYFDIWGRGTLVTVSSEIVLTQSPATLSLSPGERATLSCRASQGVSSYLAWYQQKPGQAPRLLIYGASTRATGIPARFSGSGSGTDFTLTISSLEPEDFAVYYCQQRSRWPPWTFGQGTKLEIK

SEQ ID NO: 228 - protein, 226 aa, synthetic construct
QVQLVESGGGVVQPGRSLRLSCAASGFTFSDHYMDWVRQAPGKGLEWVAIIWFDGSSKYYVDSVKGRFTISRDNSKNTLYLQMNSLRAEDTAVYYCARELGRRYFDIWGRGTLVTVSSEIVLTQSPATLSLSPGERATLSCRASQGVSSYLAWYQQKPGQAPRLLIYGASTRATGIPARFSGSGSGTDFTLTISSLEPEDFAVYYCQQRSRWPPWTFGQGTKLEIK

SEQ ID NO: 229 - protein, 226 aa, synthetic construct
QVQLVESGGGVVQPGRSLRLSCAASGFTFSDHYMDWVRQAPGKGLEWVAIIWFDGSSKYYPGSVKGRFTISRDNSKNTLYLQMNSLRAEDTAVYYCARELGRRYFDIWGRGTLVTVSSEIVLTQSPATLSLSPGERATLSCRASQGVSSYLAWYQQKPGQAPRLLIYGASTRATGIPARFSGSGSGTDFTLTISSLEPEDFAVYYCQQRSRWPPWTFGQGTKLEIK

SEQ ID NO: 230 - protein, 226 aa, synthetic construct
QVQLVESGGGVVQPGRSLRLSCAASGFTFSSYSMHWVRQAPGKGLEWVAIIWFDGSSKYYADSVKGRFTISRDNSKNTLYLQMNSLRAEDTAVYYCARELGRRYFDIWGRGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 231 - protein, 226 aa, synthetic construct
QVQLVESGGGVVQPGRSLRLSCAASGFTFSSYSMHWVRQAPGKGLEWVAIIWFDGSSKYYVDSVKGRFTISRDNSKNTLYLQMNSLRAEDTAVYYCARELGRRYFDIWGRGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 232 - protein, 226 aa, synthetic construct
QVQLVESGGGVVQPGRSLRLSCAASGFTFSSYSMHWVRQAPGKGLEWVAIIWFDGSSKYYPGSVKGRFTISRDNSKNTLYLQMNSLRAEDTAVYYCARELGRRYFDIWGRGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 233 - protein, 226 aa, synthetic construct
QVQLVESGGGVVQPGRSLRLSCAASGFTFSDYTMHWVRQAPGKGLEWVAIIWFDGSSKYYADSVKGRFTISRDNSKNTLYLQMNSLRAEDTAVYYCARELGRRYFDIWGRGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 234 - protein, 226 aa, synthetic construct
QVQLVESGGGVVQPGRSLRLSCAASGFTFSDYTMHWVRQAPGKGLEWVAIIWFDGSSKYYVDSVKGRFTISRDNSKNTLYLQMNSLRAEDTAVYYCARELGRRYFDIWGRGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 235 - protein, 226 aa, synthetic construct
QVQLVESGGGVVQPGRSLRLSCAASGFTFSDYTMHWVRQAPGKGLEWVAIIWFDGSSKYYPGSVKGRFTISRDNSKNTLYLQMNSLRAEDTAVYYCARELGRRYFDIWGRGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 236 - protein, 226 aa, synthetic construct
QVQLVESGGGVVQPGRSLRLSCAASGFTFSDHYMDWVRQAPGKGLEWVAIIWFDGSSKYYADSVKGRFTISRDNSKNTLYLQMNSLRAEDTAVYYCARELGRRYFDIWGRGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 237 - protein, 226 aa, synthetic construct
QVQLVESGGGVVQPGRSLRLSCAASGFTFSDHYMDWVRQAPGKGLEWVAIIWFDGSSKYYVDSVKGRFTISRDNSKNTLYLQMNSLRAEDTAVYYCARELGRRYFDIWGRGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 238 - protein, 226 aa, synthetic construct
QVQLVESGGGVVQPGRSLRLSCAASGFTFSDHYMDWVRQAPGKGLEWVAIIWFDGSSKYYPGSVKGRFTISRDNSKNTLYLQMNSLRAEDTAVYYCARELGRRYFDIWGRGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 239 - protein, 226 aa, synthetic construct
EVQLVESGGGVVQPGRSLRLSCAASGFTFSDYTMHWVRQAPGKGLEWVAIIWFDGSSKYYPGSVKGRFTISRDNSKNTLYLQMNSLRAEDTAVYYCARELGRRYFDIWGRGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 240 - protein, 226 aa, synthetic construct
EVQLVESGGGVVQPGRSLRLSCAASGFTFSSYSMHWVRQAPGKGLEWVAIIWFDGSNKYYADSVKSRFTISRDNSKNTLYLQMNSLRAEDTAVYYCARELGRRYFDIWGQGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 241 - protein, 226 aa, synthetic construct
EVQLVESGGGVVQPGRSLRLSCAASGFTFSSYSMHWVRQAPGKGLEWVAIIWFDGSSKYYVDSVKSRFTISRDNSKNTLYLQMNSLRAEDTAVYYCARELGRRYFDIWGQGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 242 - protein, 226 aa, synthetic construct
EVQLVESGGGVVQPGRSLRLSCAASGFTFSSYSMHWVRQAPGKGLEWVAIIWFDGSEKYYVDSVKSRFTISRDNSKNTLYLQMNSLRAEDTAVYYCARELGRRYFDIWGQGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 243 - protein, 226 aa, synthetic construct
EVQLVESGGGVVQPGRSLRLSCAASGFTFSSYSMHWVRQAPGKGLEWVAVIWYDGSSKYYADSVKGRFTISRDNSKNTLYLQMNSLRAEDTAVYYCARELGRRYFDIWGQGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 244 - protein, 226 aa, synthetic construct
EVQLVESGGGVVQPGRSLRLSCAASGFTFSSYSMHWVRQAPGKGLEWVAVIWYDGSSKYYADSVKSRFTISRDNSKNTLYLQMNSLRAEDTAVYYCARELGRRYFDIWGRGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 245 - protein, 226 aa, synthetic construct
EVQLVESGGGVVQPGRSLRLSCAASGFTFSSYSMHWVRQAPGKGLEWVAVIWYDGSSKYYADSVKSRFTISRDNSKNTLYLQMNSLRAEDTAVYYCARELGRRYFDIWGQGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 246 - protein, 226 aa, synthetic construct
EVQLVESGGGVVQPGRSLRLSCAASGFTFDDYAMHWVRQAPGKGLEWVAIIWFDGSSKYYADSVKGRFTISRDNSKNTLYLQMNSLRAEDTAVYYCARELGRRYFDIWGQGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 247 - protein, 226 aa, synthetic construct
EVQLVESGGGVVQPGRSLRLSCAASGFTFDDYTMHWVRQAPGKGLEWVAIIWFDGSSKYYADSVKGRFTISRDNSKNTLYLQMNSLRAEDTAVYYCARELGRRYFDIWGQGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 248 - protein, 226 aa, synthetic construct
EVQLVESGGGVVQPGRSLRLSCAASGFTFDDYAMHWVRQAPGKGLEWVAVIWYDGSSKYYADSVKGRFTISRDNSKNTLYLQMNSLRAEDTAVYYCARELGRRYFDIWGQGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 249 - protein, 226 aa, synthetic construct
EVQLVESGGGVVQPGRSLRLSCAASGFTFDDYTMHWVRQAPGKGLEWVAVIWYDGSSKYYADSVKGRFTISRDNSKNTLYLQMNSLRAEDTAVYYCARELGRRYFDIWGQGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 250 - protein, 226 aa, synthetic construct
EVQLVESGGGVVQPGRSLRLSCAASGFTFSDYTMHWVRQAPGKGLEWVAIIWFDGSSKYYADSVKSRFTISRDNSKNTLYLQMNSLRAEDTAVYYCARELGRRYFDIWGQGTLVTVSSEIVMTQSPATLSVSPGERATLSCRASQGVSSYLAWYQQKPGQAPRLLIYGASTRATGIPARFSGSGSGTEFTLTISSLQSEDFAVYYCQQRSRWPPWTFGQGTKLEIK

SEQ ID NO: 251 - protein, 226 aa, synthetic construct
EVQLVESGGGVVQPGRSLRLSCAASGFTFSDYTMHWVRQAPGKGLEWVAIIWFDGSSKYYPGSVKGRFTISRDNSKNTLYLQMNSLRAEDTAVYYCARELGRRYFDIWGRGTLVTVSSEIVMTQSPATLSVSPGERATLSCRASQGVSSNLAWYQQKPGQAPRLLIYGASTRATGIPARFSGSGSGTEFTLTISSLQSEDFAVYYCQQRSRWPPWTFGGGTKLEIK

SEQ ID NO: 252 - protein, 226 aa, synthetic construct
EVQLVESGGGVVQPGRSLRLSCAASGFTFSSYSMHWVRQAPGKGLEWVAIIWFDGSNKYYADSVKSRFTISRDNSKNTLYLQMNSLRAEDTAVYYCARELGRRYFDIWGQGTLVTVSSEIVMTQSPATLSVSPGERATLSCRASQGVSSNLAWYQQKPGQAPRLLIYGASTRATGIPARFSGSGSGTEFTLTISSLQSEDFAVYYCQQRSRWPPWTFGGGTKLEIK

SEQ ID NO: 253 - protein, 226 aa, synthetic construct
EVQLVESGGGVVQPGRSLRLSCAASGFTFSSYSMHWVRQAPGKGLEWVAIIWFDGSSKYYVDSVKSRFTISRDNSKNTLYLQMNSLRAEDTAVYYCARELGRRYFDIWGQGTLVTVSSEIVMTQSPATLSVSPGERATLSCRASQGVSSNLAWYQQKPGQAPRLLIYGASTRATGIPARFSGSGSGTEFTLTISSLQSEDFAVYYCQQRSRWPPWTFGGGTKLEIK

SEQ ID NO: 254 - protein, 226 aa, synthetic construct
EVQLVESGGGVVQPGRSLRLSCAASGFTFSSYSMHWVRQAPGKGLEWVAIIWFDGSEKYYVDSVKSRFTISRDNSKNTLYLQMNSLRAEDTAVYYCARELGRRYFDIWGQGTLVTVSSEIVMTQSPATLSVSPGERATLSCRASQGVSSNLAWYQQKPGQAPRLLIYGASTRATGIPARFSGSGSGTEFTLTISSLQSEDFAVYYCQQRSRWPPWTFGGGTKLEIK

SEQ ID NO: 255 - protein, 226 aa, synthetic construct
EVQLVESGGGVVQPGRSLRLSCAASGFTFSSYSMHWVRQAPGKGLEWVAVIWYDGSSKYYADSVKGRFTISRDNSKNTLYLQMNSLRAEDTAVYYCARELGRRYFDIWGQGTLVTVSSEIVMTQSPATLSVSPGERATLSCRASQGVSSNLAWYQQKPGQAPRLLIYGASTRATGIPARFSGSGSGTEFTLTISSLQSEDFAVYYCQQRSRWPPWTFGGGTKLEIK

SEQ ID NO: 256 - protein, 226 aa, synthetic construct
EVQLVESGGGVVQPGRSLRLSCAASGFTFSSYSMHWVRQAPGKGLEWVAVIWYDGSSKYYADSVKSRFTISRDNSKNTLYLQMNSLRAEDTAVYYCARELGRRYFDIWGRGTLVTVSSEIVMTQSPATLSVSPGERATLSCRASQGVSSNLAWYQQKPGQAPRLLIYGASTRATGIPARFSGSGSGTEFTLTISSLQSEDFAVYYCQQRSRWPPWTFGGGTKLEIK

SEQ ID NO: 257 - protein, 226 aa, synthetic construct
EVQLVESGGGVVQPGRSLRLSCAASGFTFSSYSMHWVRQAPGKGLEWVAVIWYDGSSKYYADSVKSRFTISRDNSKNTLYLQMNSLRAEDTAVYYCARELGRRYFDIWGQGTLVTVSSEIVMTQSPATLSVSPGERATLSCRASQGVSSNLAWYQQKPGQAPRLLIYGASTRATGIPARFSGSGSGTEFTLTISSLQSEDFAVYYCQQRSRWPPWTFGGGTKLEIK

SEQ ID NO: 258 - protein, 226 aa, synthetic construct
EVQLVESGGGVVQPGRSLRLSCAASGFTFDDYAMHWVRQAPGKGLEWVAIIWFDGSSKYYADSVKGRFTISRDNSKNTLYLQMNSLRAEDTAVYYCARELGRRYFDIWGQGTLVTVSSEIVMTQSPATLSVSPGERATLSCRASQGVSSNLAWYQQKPGQAPRLLIYGASTRATGIPARFSGSGSGTEFTLTISSLQSEDFAVYYCQQRSRWPPWTFGGGTKLEIK

SEQ ID NO: 259 - protein, 226 aa, synthetic construct
EVQLVESGGGVVQPGRSLRLSCAASGFTFDDYTMHWVRQAPGKGLEWVAIIWFDGSSKYYADSVKGRFTISRDNSKNTLYLQMNSLRAEDTAVYYCARELGRRYFDIWGQGTLVTVSSEIVMTQSPATLSVSPGERATLSCRASQGVSSNLAWYQQKPGQAPRLLIYGASTRATGIPARFSGSGSGTEFTLTISSLQSEDFAVYYCQQRSRWPPWTFGGGTKLEIK

SEQ ID NO: 260 - protein, 226 aa, synthetic construct
EVQLVESGGGVVQPGRSLRLSCAASGFTFDDYAMHWVRQAPGKGLEWVAVIWYDGSSKYYADSVKGRFTISRDNSKNTLYLQMNSLRAEDTAVYYCARELGRRYFDIWGQGTLVTVSSEIVMTQSPATLSVSPGERATLSCRASQGVSSNLAWYQQKPGQAPRLLIYGASTRATGIPARFSGSGSGTEFTLTISSLQSEDFAVYYCQQRSRWPPWTFGGGTKLEIK

SEQ ID NO: 261 - protein, 226 aa, synthetic construct
EVQLVESGGGVVQPGRSLRLSCAASGFTFDDYTMHWVRQAPGKGLEWVAVIWYDGSSKYYADSVKGRFTISRDNSKNTLYLQMNSLRAEDTAVYYCARELGRRYFDIWGQGTLVTVSSEIVMTQSPATLSVSPGERATLSCRASQGVSSNLAWYQQKPGQAPRLLIYGASTRATGIPARFSGSGSGTEFTLTISSLQSEDFAVYYCQQRSRWPPWTFGGGTKLEIK

SEQ ID NO: 262 - protein, 226 aa, synthetic construct
EVQLVESGGGVVQPGRSLRLSCAASGFTFSDYTMHWVRQAPGKGLEWVAIIWFDGSSKYYADSVKSRFTISRDNSKNTLYLQMNSLRAEDTAVYYCARELGRRYFDIWGQGTLVTVSSEIVMTQSPATLSVSPGERATLSCRASQGVSSNLAWYQQKPGQAPRLLIYGASTRATGIPARFSGSGSGTEFTLTISSLQSEDFAVYYCQQRSRWPPWTFGGGTKLEIK

SEQ ID NO: 263 - protein, 5 aa, synthetic construct
SYGMH

SEQ ID NO: 264 - protein, 5 aa, synthetic construct
HYGMH

SEQ ID NO: 265 - protein, 5 aa, synthetic construct
SHGMH

SEQ ID NO: 266 - protein, 5 aa, synthetic construct
SYHMH

SEQ ID NO: 267 - protein, 5 aa, synthetic construct
SYGHH

SEQ ID NO: 268 - protein, 5 aa, synthetic construct
DYAMH

SEQ ID NO: 269 - protein, 5 aa, synthetic construct
DYYMS

SEQ ID NO: 270 - protein, 5 aa, synthetic construct
DYGMS

SEQ ID NO: 271 - protein, 5 aa, synthetic construct
DYTMH

SEQ ID NO: 272 - protein, 5 aa, synthetic construct
DHYMD

SEQ ID NO: 273 - protein, 5 aa, synthetic construct
SYDMH

SEQ ID NO: 274 - protein, 5 aa, synthetic construct
SYSMH

SEQ ID NO: 275 - protein, 5 aa, synthetic construct
SYSMN

SEQ ID NO: 276 - protein, 5 aa, synthetic construct
DYAMS

SEQ ID NO: 277 - protein, 17 aa, synthetic construct
IIWFDGSSTYYADSVRG

SEQ ID NO: 278 - protein, 17 aa, synthetic construct
HIWFDGSSTYYADSVRG

SEQ ID NO: 279 - protein, 17 aa, synthetic construct
IHWFDGSSTYYADSVRG

SEQ ID NO: 280 - protein, 17 aa, synthetic construct
IIHFDGSSTYYADSVRG

SEQ ID NO: 281 - protein, 17 aa, synthetic construct
IIWHDGSSTYYADSVRG

SEQ ID NO: 282 - protein, 17 aa, synthetic construct
IIWFHGSSTYYADSVRG

SEQ ID NO: 283 - protein, 17 aa, synthetic construct
IIWFDHSSTYYADSVRG

SEQ ID NO: 284 - protein, 17 aa, synthetic construct
IIWFDGHSTYYADSVRG

SEQ ID NO: 285 - protein, 17 aa, synthetic construct
IIWFDGSHTYYADSVRG

SEQ ID NO: 286 - protein, 17 aa, synthetic construct
IIWFDGSSHYYADSVRG

SEQ ID NO: 287 - protein, 17 aa, synthetic construct
IIWFDGSSTHYADSVRG

SEQ ID NO: 288 - protein, 17 aa, synthetic construct
IIWFDGSSTYHADSVRG

SEQ ID NO: 289 - protein, 17 aa, synthetic construct
IIWFDGSSTYYHDSVRG

SEQ ID NO: 290 - protein, 17 aa, synthetic construct
IIWFDGSSTYYAHSVRG

SEQ ID NO: 291 - protein, 17 aa, synthetic construct
IIWFDGSSTYYAHSVRG

SEQ ID NO: 292 - protein, 17 aa, synthetic construct
IIWFDGSSTYYADSHRG

SEQ ID NO: 293 - protein, 17 aa, synthetic construct
IIWFDGSSTYYADSVHG

SEQ ID NO: 294 - protein, 17 aa, synthetic construct
IIWFDGSSTYYADSVRH

SEQ ID NO: 295 - protein, 17 aa, synthetic construct
IIWFDGSSHYYADSVKG

SEQ ID NO: 296 - protein, 17 aa, synthetic construct
IIWFDGSSKYYADSVKG

SEQ ID NO: 297 - protein, 17 aa, synthetic construct
IIWFDGSSHYYVDSVKG

SEQ ID NO: 298 - protein, 17 aa, synthetic construct
IIWFDGSSKYYVDSVKG

SEQ ID NO: 299 - protein, 17 aa, synthetic construct
IIWFDGSSHYYPGSVKG

SEQ ID NO: 300 - protein, 17 aa, synthetic construct
IIWFDGSSKYYPGSVKG

SEQ ID NO: 301 - protein, 17 aa, synthetic construct
VIWYDGSSHYYADSVKG

SEQ ID NO: 302 - protein, 17 aa, synthetic construct
VIWYDGSSKYYADSVKG

SEQ ID NO: 303 - protein, 17 aa, synthetic construct
VIWYDGSSHYYVDSVKG

SEQ ID NO: 304 - protein, 17 aa, synthetic construct
VIWYDGSSKYYVDSVKG

SEQ ID NO: 305 - protein, 17 aa, synthetic construct
VIWYDGSSHYYPGSVKG

SEQ ID NO: 306 - protein, 17 aa, synthetic construct
VIWYDGSSKYYPGSVKG

SEQ ID NO: 307 - protein, 17 aa, synthetic construct
IIWFDGSNKYYADSVKS

SEQ ID NO: 308 - protein, 17 aa, synthetic construct
IIWFDGSSKYYVDSVKS

SEQ ID NO: 309 - protein, 17 aa, synthetic construct
IIWFDGSEKYYVDSVKS

SEQ ID NO: 310 - protein, 17 aa, synthetic construct
IIWFDGSSKYYADSVKS

SEQ ID NO: 311 - protein, 9 aa, synthetic construct
ELGRRYFDL

SEQ ID NO: 312 - protein, 9 aa, synthetic construct
HLGRRYFDL

SEQ ID NO: 313 - protein, 9 aa, synthetic construct
EHGRRYFDL

SEQ ID NO: 314 - protein, 9 aa, synthetic construct
ELHRRYFDL

SEQ ID NO: 315 - protein, 9 aa, synthetic construct
ELGHRYFDL

SEQ ID NO: 316 - protein, 9 aa, synthetic construct
ELGRHYFDL

SEQ ID NO: 317 - protein, 9 aa, synthetic construct
ELGRRHFDL

SEQ ID NO: 318 - protein, 9 aa, synthetic construct
ELGRRYHDL

SEQ ID NO: 319 - protein, 9 aa, synthetic construct
ELGRRYFHL

SEQ ID NO: 320 - protein, 9 aa, synthetic construct
ELGRRYFDH

SEQ ID NO: 321 - protein, 9 aa, synthetic construct
ELGRRYFDI

SEQ ID NO: 322 - protein, 11 aa, synthetic construct
RASQSVSSYLA

SEQ ID NO: 323 - protein, 11 aa, synthetic construct
HASQSVSSYLA

SEQ ID NO: 324 - protein, 11 aa, synthetic construct
RHSQSVSSYLA

SEQ ID NO: 325 - protein, 11 aa, synthetic construct
RAHQSVSSYLA

SEQ ID NO: 326 - protein, 11 aa, synthetic construct
RASHSVSSYLA

SEQ ID NO: 327 - protein, 11 aa, synthetic construct
RASQHVSSYLA

SEQ ID NO: 328 - protein, 11 aa, synthetic construct
RASQSHSSYLA

SEQ ID NO: 329 - protein, 11 aa, synthetic construct
RASQSVHSYLA

SEQ ID NO: 330 - protein, 11 aa, synthetic construct
RASQSVSHYLA

SEQ ID NO: 331 - protein, 11 aa, synthetic construct
RASQSVSSHLA

SEQ ID NO: 332 - protein, 11 aa, synthetic construct
RASQSVSSYHA

SEQ ID NO: 333 - protein, 11 aa, synthetic construct
RASQSVSSYLH

SEQ ID NO: 334 - protein, 11 aa, synthetic construct
RASQSVSSNLA

SEQ ID NO: 335 - protein, 12 aa, synthetic construct
RASQSVSSSYLA

SEQ ID NO: 336 - protein, 11 aa, synthetic construct
RASQGVSSYLA

SEQ ID NO: 337 - protein, 12 aa, synthetic construct
GASQSVSSSYLA

SEQ ID NO: 338 - protein, 12 aa, synthetic construct
RASQSVSSSYLS

SEQ ID NO: 339 - protein, 7 aa, synthetic construct
DASKRAT

SEQ ID NO: 340 - protein, 7 aa, synthetic construct
HASKRAT

SEQ ID NO: 341 - protein, 7 aa, synthetic construct
DHSKRAT

SEQ ID NO: 342 - protein, 7 aa, synthetic construct
DAHKRAT

SEQ ID NO: 343 - protein, 7 aa, synthetic construct
DASHRAT

SEQ ID NO: 344 - protein, 7 aa, synthetic construct
DASKHAT

SEQ ID NO: 345 - protein, 7 aa, synthetic construct
DASKRHT

SEQ ID NO: 346 - protein, 7 aa, synthetic construct
DASKRAH

SEQ ID NO: 347 - protein, 7 aa, synthetic construct
DASNRAT

SEQ ID NO: 348 - protein, 7 aa, synthetic construct
GASTRAT

SEQ ID NO: 349 - protein, 7 aa, synthetic construct
GASSRAT

SEQ ID NO: 350 - protein, 7 aa, synthetic construct
DASSRAT

SEQ ID NO: 351 - protein, 10 aa, synthetic construct
QQRSKWPPWT

SEQ ID NO: 352 - protein, 10 aa, synthetic construct
HQRSKWPPWT

SEQ ID NO: 353 - protein, 10 aa, synthetic construct
QHRSKWPPWT

SEQ ID NO: 354 - protein, 10 aa, synthetic construct
QQHSKWPPWT

SEQ ID NO: 355 - protein, 10 aa, synthetic construct
QQRHKWPPWT

SEQ ID NO: 356 - protein, 10 aa, synthetic construct
QQRSHWPPWT

SEQ ID NO: 357 - protein, 10 aa, synthetic construct
QQRSKHPPWT

SEQ ID NO: 358 - protein, 10 aa, synthetic construct
QQRSKWHPWT

SEQ ID NO: 359 - protein, 10 aa, synthetic construct
QQRSKWPHWT

SEQ ID NO: 360 - protein, 10 aa, synthetic construct
QQRSKWPPHT

SEQ ID NO: 361 - protein, 10 aa, synthetic construct
QQRSKWPPWH

SEQ ID NO: 362 - protein, 10 aa, synthetic construct
QQRSRWPPWT

SEQ ID NO: 363 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 364 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 365 - protein, 215 aa, synthetic construct
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 366 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 367 - protein, 330 aa, synthetic construct [sequence omitted]

SEQ ID NO: 368 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 369 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 370 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371 - protein, 454 aa, synthetic construct
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 372 - protein, 453 aa, synthetic construct
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 373 - protein, 453 aa, synthetic construct
QVQLVQSGAEVVKPGASVKLSSKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 374 - protein, 219 aa, synthetic construct
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 375 - protein, 5 aa, synthetic construct
GGGGS

SEQ ID NO: 376 - protein, 5 aa, synthetic construct
GGGGA

SEQ ID NO: 377 - protein, 215 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 378 - protein, 448 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 379 - protein, 663 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 380 - protein, 219 aa, synthetic construct
DIVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 381 - protein, 447 aa, synthetic construct
QVQLQESGPGLVKPSETLSLTCTVSGYSITGGYLWNWIRQPPGKGLEWIGYISYDGTNNYKPSLKDRVTISRDTSKNQFSLKLSSVTAADTAVYYCARYGRVFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 382 - protein, 666 aa, synthetic construct
DIVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLQESGPGLVKPSETLSLTCTVSGYSITGGYLWNWIRQPPGKGLEWIGYISYDGTNNYKPSLKDRVTISRDTSKNQFSLKLSSVTAADTAVYYCARYGRVFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 383 - protein, 214 aa, synthetic construct
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 384 - protein, 460 aa, synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 385 - protein, 674 aa, synthetic construct
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECSEVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 386 - protein, 214 aa, synthetic construct
DIQMTQFPSSLSASVGDRVTITCRASQGIRNDLGWYQQKPGKAPKRLIYAASRLHRGVPSRFSGSGSGTEFTLTISSLQPEDFATYYCLQHNSYPCSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 387 - protein, 450 aa, synthetic construct
EVQLLESGGGLVQPGGSLRLSCTASGFTFSSYAMNWVRQAPGKGLEWVSAISGSGGTTFYADSVKGRFTISRDNSRTTLYLQMNSLRAEDTAVYYCAKDLGWSDSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 388 - protein, 664 aa, synthetic construct
DIQMTQFPSSLSASVGDRVTITCRASQGIRNDLGWYQQKPGKAPKRLIYAASRLHRGVPSRFSGSGSGTEFTLTISSLQPEDFATYYCLQHNSYPCSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLLESGGGLVQPGGSLRLSCTASGFTFSSYAMNWVRQAPGKGLEWVSAISGSGGTTFYADSVKGRFTISRDNSRTTLYLQMNSLRAEDTAVYYCAKDLGWSDSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 389 - protein, 219 aa, synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 390 - protein, 449 aa, synthetic construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARWTGRTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 391 - protein, 668 aa, synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARWTGRTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 392 - protein, 214 aa, synthetic construct
EIVLTQSPGTLSVSPGERATLSCRASQSIGSSLHWYQQKPGQAPRLLIKYASQSLSGIPDRFSGSGSGTDFTLTISRLEPEDFAVYYCHQSSRLPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 393 - protein, 448 aa, synthetic construct
EVQLVQSGGGLVKPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 394 - protein, 662 aa, synthetic construct
EIVLTQSPGTLSVSPGERATLSCRASQSIGSSLHWYQQKPGQAPRLLIKYASQSLSGIPDRFSGSGSGTDFTLTISRLEPEDFAVYYCHQSSRLPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVQSGGGLVKPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 395 - protein, 446 aa, synthetic construct
QVQLQESGPGLVKPSETLSLTCTVSGYSITGGYLWNWIRQPPGKGLEWIGYISYDGTNNYKPSLKDRVTISRDTSKNQFSLKLSSVTAADTAVYYCARYGRVFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 396 - protein, 665 aa, synthetic construct
DIVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLQESGPGLVKPSETLSLTCTVSGYSITGGYLWNWIRQPPGKGLEWIGYISYDGTNNYKPSLKDRVTISRDTSKNQFSLKLSSVTAADTAVYYCARYGRVFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 397 - protein, 17 aa, synthetic construct
VIWYDGSSKYYADSVKS